(19) United States
(12) Patent Application Publication
(10) Pub. No.: US 2013/0143955 A1
(43) Pub. Date: Jun. 6, 2013

(54) Cyclic di-GMP-II Riboswitches, Motifs, and Compounds, and Methods for Their Use

(71) Applicants: Ronald R. Breaker, Guilford, CT (US); Jenny Baker, Churchville, NY (US); Elaine Lee, Covington, WA (US); Narasimhan Sudarsan, New Haven, CT (US); Zasha Weinberg, New Haven, CT (US)

(72) Inventors: Ronald R. Breaker, Guilford, CT (US); Jenny Baker, Churchville, NY (US); Elaine Lee, Covington, WA (US); Narasimhan Sudarsan, New Haven, CT (US); Zasha Weinberg, New Haven, CT (US)

(73) Assignee: YALE UNIVERSITY, New Haven, CT (US)

(21) Appl. No.: 13/816,088

(22) PCT Filed: Aug. 9, 2011
(86) PCT No.: PCT/US11/47143

§ 371 Date: Feb. 8, 2013

Related U.S. Application Data
(60) Provisional application No. 61/371,915, filed on Aug. 9, 2010.

(51) Int. Cl. A01N 57/16; A61K 48/00; A61K 31/7084; C07H 21/04; A61K 38/16; C12N 15/72

(52) U.S. Cl. CPC A01N 57/16; A61K 31/7084
USPC 514/44 R; 536/23.1; 435/29; 536/24.1; 435/6.12

(57) ABSTRACT

Disclosed are compositions and methods involving cyclic di-GMP—responsive Riboswitches and cyclic di-GMP-II motifs.

### CROSS-REFERENCE TO RELATED APPLICATIONS

This application claims benefit of U.S. Provisional Application No. 61/371,915, filed Aug. 9, 2010. U.S. Provisional Application No. 61/371,915, filed Aug. 9, 2010, is hereby incorporated herein by reference in its entirety.

### STATEMENT REGARDING FEDERALLY SPONSORED RESEARCH

This invention was made with government support under Grant No. GM022778 awarded by the National Institutes of Health (NIH). The government has certain rights in the invention.

### REFERENCE TO SEQUENCE LISTING

The Sequence Listing submitted Aug. 9, 2011 as a text file named “YU_{—}5362_PCT_AMD_AFD_Sequence_Listing.txt,” created on Aug. 9, 2011, and having a size of 57,880 bytes is hereby incorporated by reference pursuant to 37 C.F.R. §1.52(e)(5).

### FIELD OF THE INVENTION

The disclosed invention is generally in the fields of gene expression and antimicrobial compounds, and specifically in the area of regulation of gene expression and targeting of gene expression with antimicrobial compounds.

### BACKGROUND OF THE INVENTION

Precision genetic control is an essential feature of living systems, as cells must respond to a multitude of biochemical signals and environmental cues by varying genetic expression patterns. Most known mechanisms of genetic control involve the use of protein factors that sense chemical or physical stimuli and then modulate gene expression by selectively interacting with the relevant DNA or messenger RNA sequence. Proteins can adopt complex shapes and carry out a variety of functions that permit living systems to sense accurately their chemical and physical environments. Protein factors that respond to metabolites typically act by binding DNA to modulate transcription initiation (e.g. the lac repressor protein; Matthews, K. S., and Nichols, J. C., 1998, Prog. Nucleic Acids Res. Mol. Biol. 58, 127-164) or by binding RNA to control either transcription termination (e.g. the PyrR protein; Switzer, R. L., et al., 1999, Prog. Nucleic Acids Res. Mol. Biol. 62, 329-367) or translation (e.g. the TRAP protein; Babitzke, P., and Gollnick, P., 2001, J. Bacteriol. 183, 5795-5802). Protein factors respond to environmental stimuli by various mechanisms such as allosteric modulation or post-translational modification, and are adept at exploiting these mechanisms to serve as highly responsive genetic switches (e.g. see Ptashne, M., and Gann, A. (2002). Genes and Signals. Cold Spring Harbor Laboratory Press, Cold Spring Harbor, N.Y.).

In addition to the widespread participation of protein factors in genetic control, it is also known that RNA can take an active role in genetic regulation. Recent studies have begun to reveal the substantial role that small non-coding RNAs play in selectively targeting mRNAs for destruction, which results in down-regulation of gene expression (e.g. see Hannon, G. J. 2002, Nature 418, 244-251 and references therein). This process of RNA interference takes advantage of the ability of short RNAs to recognize the intended mRNA target selectively via Watson-Crick base complementation, after which the bound mRNAs are destroyed by the action of proteins. RNAs are ideal agents for molecular recognition in this system because it is far easier to generate new target-specific RNA factors through evolutionary processes than it would be to generate protein factors with novel but highly specific RNA binding sites.

Although proteins fulfill most requirements that biology has for enzyme, receptor and structural functions, RNA also can serve in these capacities. For example, RNA has sufficient structural plasticity to form numerous ribozyme domains (Cech & Golden, Building a catalytic active site using only RNA. In: The RNA World R. F. Gesteland, T. R. Cech, J. F. Atkins, eds., pp. 321-350 (1998); Breaker, In vitro selection of catalytic polynucleotides. Chem. Rev. 97, 371-390 (1997)) and receptor domains (Osborne & Ellington, Nucleic acid selection and the challenge of combinatorial chemistry. Chem. Rev. 97, 349-370 (1997); Hermann & Patel, Adaptive recognition by nucleic acid aptamers. Science 287, 820-825 (2000)) that exhibit considerable enzymatic power and precise molecular recognition. Furthermore, these activities can be combined to create allosteric ribozymes (Soukup & Breaker, Engineering precision RNA molecular switches. Proc. Natl. Acad. Sci. USA 96, 3584-3589 (1999); Seetharaman et al., Immobilized riboswitches for the analysis of complex chemical and biological mixtures. Nature Biotechnol. 19, 336-341 (2001)) that are selectively modulated by effector molecules.

Riboswitches are genetic control elements embodied in RNA transcripts that regulate expression of the transcripts in which they are located. Riboswitches are often located within the 5′-untranslated region (5′-UTR) of the main coding region of a particular mRNA. Riboswitches are genetic regulatory elements composed solely of RNA that bind metabolites and control gene expression commonly without the involvement of protein factors (Breaker R R. Riboswitches: from ancient gene-control systems to modern drug targets. Future Microbiol 2009; 4:771-773).

### BRIEF SUMMARY OF THE INVENTION

Disclosed are methods and compositions for altering gene expression of genes by affecting cyclic di-GMP-responsive riboswitches operably linked to the genes, where the riboswitch comprises a cyclic di-GMP-II motif. For example, the methods can comprise bringing into contact a compound and a cell, where the compound affects the riboswitch. The cell can comprise a gene encoding an RNA comprising a cyclic di-GMP-responsive riboswitch. The riboswitch can comprise a cyclic di-GMP-II motif.

In some forms, the cell can have been identified as being in need of altered gene expression. In some forms, the cell can be a bacterial cell. In some forms, the cell can be a Clostridium, Deinococcus, or Bacillus cell. In some forms, the compound kills or inhibits the growth of the bacterial cell. In some forms, the compound and the cell can be brought into contact by administering the compound to a subject. In some forms, the cell is a bacterial cell in the subject and the compound kills or inhibits the growth of the bacterial cell. In some forms, the subject has a bacterial infection. In some forms, the compound can be administered in combination with another antimicrobial compound. In some forms, the compound inhibits bacterial growth in a biofilm.

Also disclosed are regulatable gene expression constructs comprising cyclic di-GMP-responsive riboswitches operably linked to coding regions, where the riboswitch comprises a cyclic di-GMP-II motif. For example, the disclosed constructs can comprise a nucleic acid molecule encoding an RNA comprising a riboswitch operably linked to a coding region, where the riboswitch regulates expression of the RNA, where the riboswitch and coding region are heterologous, and where the riboswitch is a cyclic di-GMP-responsive riboswitch, where the riboswitch comprises a cyclic di-GMP-II motif. Also disclosed are riboswitches where the riboswitch is a non-natural derivative of a naturally-occurring a cyclic di-GMP-responsive riboswitch, where the naturally-occurring riboswitch comprises a cyclic di-GMP-II motif. Also disclosed are riboswitch ribozymes comprising a riboswitch aptamer domain operably linked to a self-splicing ribozyme, where the aptamer is comprised of the cyclic di-GMP-II motif.

In some forms, the riboswitch can comprise an aptamer domain and an expression platform domain, where the aptamer domain and the expression platform domain are heterologous, where the aptamer is comprised of the cyclic di-GMP-II motif. In some forms, the riboswitch can comprise two or more aptamer domains and an expression platform domain, where at least one of the aptamer domains and the expression platform domain are heterologous, where at least one of the aptamer domains is comprised of the cyclic di-GMP-II motif. In some forms, at least two of the aptamer domains can exhibit cooperative binding. In some forms, the riboswitch can comprise the consensus structure of FIG. 1A or 5.

In some forms, the riboswitch can comprise an aptamer domain and an expression platform domain, where the aptamer domain is derived from a naturally-occurring cyclic di-GMP-responsive riboswitch. In some forms, the aptamer domain can be the aptamer domain of a naturally-occurring cyclic di-GMP-responsive riboswitch. In some forms, the aptamer domain can have the consensus structure of an aptamer domain of the naturally-occurring riboswitch. In some forms, the aptamer domain can consist of only base pair conservative changes of the naturally-occurring riboswitch.

In some forms, the aptamer domain can comprise a P1 stem, where the P1 stem comprises an aptamer strand and a control strand, where the expression platform domain comprises a regulated strand, and where the regulated strand, the control strand, or both have been designed to form a stem structure. In some forms, the aptamer domain can comprise a control stem, where the control stem comprises an aptamer strand and a control strand, where the expression platform domain comprises a regulated strand, and where the regulated strand, the control strand, or both have been designed to form a stem structure.

In some forms, the riboswitch can comprise an aptamer domain and an expression platform domain, where the aptamer domain and the expression platform domain are heterologous, and where the aptamer is comprised of the cyclic di-GMP-II motif. In some forms, the riboswitch can be activated by a trigger molecule, where the riboswitch produces a signal when activated by the trigger molecule.

In some forms, the aptamer domain can comprise a control stem, where the control stem comprises an aptamer strand and a control strand, where the ribozyme comprises a regulated strand, and where the regulated strand, the control strand, or both have been designed to form a stem structure. In some forms, the aptamer domain and the ribozyme can be heterologous. In some forms, the riboswitch ribozyme can be operatively linked to a coding region, where the riboswitch ribozyme and the coding region are heterologous.

Also disclosed are methods and compositions for detecting a compound of interest. The method can comprise bringing into contact a sample and a riboswitch, where the riboswitch produces a signal when the sample contains the compound of interest. The riboswitch can be activated by the compound of interest and the riboswitch produces a signal when activated by the compound of interest. The riboswitch is a cyclic di-GMP-responsive riboswitch, where the riboswitch comprises a cyclic di-GMP-II motif.

In some forms, the riboswitch can change conformation when activated by the compound of interest, where the change in conformation produces a signal via a conformation dependent label. In some forms, the riboswitch can change conformation when activated by the compound of interest, where the change in conformation causes a change in expression of an RNA linked to the riboswitch, and where the change in expression produces a signal. In some forms, the signal can be produced by a reporter protein expressed from the RNA linked to the riboswitch.

Also disclosed are methods comprising (a) testing a compound for altering gene expression of a gene encoding an RNA comprising a riboswitch, and (b) altering gene expression by bringing into contact a cell and a compound that altered gene expression in step (a). The alteration can be via the riboswitch. The riboswitch is a cyclic di-GMP-responsive riboswitch, where the riboswitch comprises a cyclic di-GMP-II motif. The cell can comprise a gene encoding an RNA comprising a riboswitch, where the compound inhibits expression of the gene by binding to the riboswitch.

Also disclosed are methods and compositions for identifying riboswitches. The method can comprise assessing in-line spontaneous cleavage of an RNA molecule in the presence and absence of a compound, where the RNA molecule is encoded by a gene regulated by the compound, where a change in the pattern of in-line spontaneous cleavage of the RNA molecule indicates a riboswitch, where the RNA comprises a cyclic di-GMP-responsive riboswitch or a derivative of a cyclic di-GMP-responsive riboswitch. The riboswitch can comprise a cyclic di-GMP-II motif and the compound can be cyclic di-GMP.

Additional advantages of the disclosed method and compositions will be set forth in part in the description which follows, and in part will be understood from the description, or can be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIGS. 1A, 1B, 1C, 1D, and 1E show c-di-GMP-II riboswitches. (A) Consensus sequence and secondary structure model for c-di-GMP-II riboswitch aptamers (SEQ ID NOs:49, 50, and 52). Nucleotides in circles are conserved in >97% of the representatives. Other annotations are described in FIG. 5. (B) Wild-type (WT) 84 Cd RNA encompassing the motif upstream of a possible virulence gene (SEQ ID NO:52). Disruptive (M2) and restorative (M3) mutations are depicted, and sites of spontaneous cleavage are derived from C. Asterisks identify nucleotides added to facilitate in vitro transcription. (C) Denaturing polyacrylamide gel electrophoresis (PAGE) of WT 84 Cd RNA in-line probing cleavage products. No reaction (NR); partial digestion with ribonuclease (RNase) T1 (T1, cleaves after G residues); alkali (⁻OH, cleaves at all linkages); incubation in the absence (−) or presence (+) of 10 mM c-di-GMP. Selected RNase T1 digestion product bands are identified. (D) Plot of the fraction of RNA remaining unbound versus the logarithm of the concentration of c-di-GMP present during in-line probing. (E) K_{D} values for 84 Cd binding to c-di-GMP and various di- and mononucleotide analogs. A, adenosine; G, guanosine.

FIGS. 2A, 2B, 2C, and 2D show gene control by c-di-GMP-II riboswitches from B. halodurans and C. difficile. (A) Wild-type (WT; SEQ ID NO:53), J1/2 mutant (M1) or stem P3 mutants (M2 and M3) of the riboswitch from the B. halodurans BH0631 locus were fused to a β-galactosidase reporter gene and introduced into Bacillus subtilis for analysis. (B) Plot of the normalized level of β-galactosidase activity from cells carrying the WT, M1, M2 or M3 reporter constructs depicted in A. A value of 1 equals 157 Miller units. (C) Wild-type (WT; SEQ ID NO:54) or stem P3 mutants (M1 and M2) of the riboswitch from the ompR/baeS operon from C. difficile. Brackets identify the 146-nucleotide marker transcript (M) in D. (D) Second messenger-dependent transcription termination assays using WT and various mutant riboswitches as noted in C. FL, Term, and M identify the full-length, terminated, and marker transcripts, respectively.

FIGS. 3A, 3B, 3C, 3D, and 3E show the architecture, mechanism, and activity of a tandem riboswitch-ribozyme. (A) Structural features of the conjoined c-di-GMP-II aptamer and group I ribozyme from C. difficile, and the importance of aptamer function in RNA self-splicing (SEQ ID NO:55). Two alternative base pair interactions proposed to be important for allosteric function are outlined with alternating dashes and dots. (B) PAGE separation of products generated by self-splicing assays. NR, no reaction; C-IVS and L-IVS, circular and linear intervening sequences, respectively; Pre, 864-nucleotide precursor RNA (FIG. 6); 5′E-3′E, spliced exons; 3T*, 3′ fragment generated by GTP attack at an alternative site; Fragments designate additional RNA products presumably created by IVS circularization. Data for all mutants is not shown. (C) Tandem c-di-GMP-II aptamer and group I ribozyme arrangement. (D) Key features of the aptamer-ribozyme system, including validated splice and GTP attack sites (SEQ ID NOs:55, 56, and 57). Alternative base-pair interactions guiding allosteric function are enclosed in a dashed line oval and a alternating dash dot line oval. (E) PAGE separation of products generated by self-splicing assays. NR, no reaction; C-IVS and L-IVS, circular and linear intervening sequences, respectively; Pre, 864-nucleotide precursor RNA (FIG. 6); 5′E-3′E, spliced exons; 3′E*, 3′ fragment generated by GTP attack at an alternative site; Fragments designate additional RNA products presumably created by IVS circularization.

FIGS. 4A, 4B, 4C, 4D and 4E show the rate constant modulation by c-di-GMP. (A) Time course of [α-³²P]GTP attack at sites GTPA or GTP2 in the absence or presence of c-di-GMP. (B) Plot of the natural logarithm of the fraction of unprocessed or differently processed RNA (pre-dp RNA) versus time for the reaction in (A). Values for fraction processed were corrected for ˜50% of the precursor remaining after exhaustive incubation. (C) Time course of the production of spliced exons (5′E-3′E) or alternative GTP2 site fragment (3′E*) in the absence or presence of c-di-GMP Annotations are as described for FIG. 2C. (D) Plot of the natural logarithm of the fraction of unprocessed or differently processed RNA versus time for the reaction in (C), corrected as described in (B). (E) Changes in splice product yields on introduction of c-di-GMP. (Left) Ratio of the number of 3′E* molecules versus the number of 5′E-3′E spliced exon molecules in the absence or presence of c-di-GMP, respectively. (Right) Ratio of the numbers of 3′E* molecules and the numbers of 5′E-3′E spliced exon molecules in the absence or presence of c-di-GMP, respectively.

FIG. 5 shows comparison of the consensus sequences and structural models for class I (left; SEQ ID NOs:58 and 59) and class II (right; SEQ ID NOs:49, 50, and 51) c-di-GMP aptamers. Consensus model for c-di-GMP-I is as reported in Smith et al. 2009. The internal bulge between P2 and P3 of c-di-GMP-II aptamers conforms to a kink-turn motif (Klein et al. 2001; Winkler et al. 2001).

FIG. 6 shows the sequence and secondary structure model for the 5′ UTR and a portion of the ORF containing the 84 Cd aptamer and group I intron upstream of the gene at the CD3246 locus (SEQ ID NOs:122). The ribozyme has all the hallmarks of a typical group I ribozyme, including stems P1 through P10, and a U-G wobble base pair that defines a typical 5′ splice site (5′ SS). Nucleotides comprising the putative atypical start codon (UUG) for the associated ORF are depicted in circles. Other annotations are as described in and for FIG. 3D in the main text. This 864-nucleotide tandem construct was used for most in vitro splicing assays.

FIG. 7 is a c-di-GMP-dependent dose-response curve showing fraction of RNA fully spliced (5′E-3′E) vs log c (cyclic-di-GMP, M). The curve is for generation of ligated exons (5′E-3′E) by 5′ ³²P-labeled 864 Cd Tandem RNA incubated for 120 minutes in the presence of various concentrations of the second messenger. The S₅₀ value is the concentration of c-di-GMP required to half-maximally stimulate production of ligated exons. The line represents an expected curve for a 1-to-1 interaction between ligand and allosteric ribozyme.

FIGS. 8A, 8B, 8C, and 8D show in-line probing analysis of alternative base-paired structures that may be responsible for allosteric control. (A) Sequence and secondary structure model (SEQ ID NO:60). The structure depicted is the expected c-di-GMP ligand-bound state, while the nucleotides enclosed in dashed line ovals identify the alternative base-paired structure that would compete with formation of both the aptamer and ribozyme P1 stems. Sites of spontaneous cleavage, including sites of structure modulation, are noted with circles, diamonds, and squared enclosing nucleotides. Annotations are as described in the brief description to FIGS. 1A and 5. (B, C, D) Similar analyses of mutant RNAs as indicated (SEQ ID NOs:61, 62, and 63, respectively).

FIGS. 9A and 9B show the junctions of ribozyme self-circularization products. (A) Products due to circularization after GTP attack at the 5′ SS (GTP₁), followed by 5′ exon attack at the 3′ SS (nucleotide 667) or one nucleotide downstream (nucleotide 668). SEQ ID NOs:64, 65, and 66. (B) Products due to circularization after GTP attack at nucleotide 670, which corresponds to the alternative attack site (GTP₂). SEQ ID NOs:67, 68, 69, and 70. Nucleotides at the junctions are numbered, and the number of representatives of each circularization product observed among 19 clones sequenced are noted in parentheses.

FIGS. 10A, 10B, and 10C show the proposed mechanism for allosteric ribozyme-mediated gene control. (A) Precursor mRNA with the start codon sequestered by the ribozyme P10 stem (SEQ ID NOs:72 and 73). (B) RNA processed in the presence of GTP and c-di-GMP unmasks the start codon and creates a perfect ribosome binding site (SEQ ID NO:74). (C) RNA processed in the presence of GTP alone lacks a ribosome binding site (SEQ ID NO:75).

### DETAILED DESCRIPTION OF THE INVENTION

The disclosed methods, compounds, and compositions can be understood more readily by reference to the following detailed description of particular embodiments and the Examples included therein and to the Figures and their previous and following description.

Described herein are the interactions of two small RNA molecules and two larger RNA molecules that together influence the function of a self-splicing ribozyme, a structure many biologists had believed had no role other than to reproduce itself. However, in the pathogenic stomach bacterium Clostridium difficile, this RNA structure acts as a sort of sensor to help regulate the expression of genes, probably to help the bacterium manipulate human cells. The disclosed compositions and methods relate to a new class of riboswitches that sense the bacterial second messenger cyclic di-GMP. In the pathogen Clostridium difficile, three of these riboswitches are present. One resides adjacent to a group I self-splicing ribozyme, and the biochemical data described herein prove this RNA architecture represents the first natural example of an allosteric ribozyme. Ligand binding controls splicing of an mRNA for a virulence gene. Therefore, this new riboswitch class is a useful drug target for novel antibiotics.

Messenger RNAs are typically thought of as passive carriers of genetic information that are acted upon by protein- or small RNA-regulatory factors and by ribosomes during the process of translation. It was discovered that certain mRNAs carry natural aptamer domains and that binding of specific metabolites directly to these RNA domains leads to modulation of gene expression. In particular, it as been discovered certain cyclic di-GMP-responsive riboswitches comprise a cyclic di-GMP-II motif. Natural riboswitches exhibit two surprising functions that are not typically associated with natural RNAs. First, the mRNA element can adopt distinct structural states wherein one structure serves as a precise binding pocket for its target metabolite. Second, the metabolite-induced allosteric interconversion between structural states causes a change in the level of gene expression by one of several distinct mechanisms. Riboswitches typically can be dissected into two separate domains: one that selectively binds the target (aptamer domain) and another that influences genetic control (expression platform). It is the dynamic interplay between these two domains that results in metabolite-dependent allosteric control of gene expression.

Distinct classes of riboswitches have been identified and are shown to selectively recognize activating compounds (referred to herein as trigger molecules). For example, coenzyme B₁₂, glycine, thiamine pyrophosphate (TPP), and flavin mononucleotide (FMN) activate riboswitches present in genes encoding key enzymes in metabolic or transport pathways of these compounds. The aptamer domain of each riboswitch class conforms to a highly conserved consensus sequence and structure. Thus, sequence homology searches can be used to identify related riboswitch domains. Riboswitch domains have been discovered in various organisms from bacteria, archaea, and eukarya.

Riboswitches are genetic regulatory elements composed solely of RNA that bind metabolites and control gene expression commonly without the involvement of protein factors (Breaker R R. Riboswitches: from ancient gene-control systems to modern drug targets. Future Microbiol 2009; 4:771-773). Most simple riboswitches are composed of an aptamer domain and an expression platform, where the aptamer functions as a receptor for a specific metabolite and the expression platform modulates the expression of one or more genes in a ligand-dependent fashion (Barrick et al. The distributions, mechanisms, and structures of metabolite-binding riboswitches. Genome Biol 2007; 8:R239; Dambach et al. Expanding roles for metabolite-sensing regulatory RNAs. Curr Opin Microbiol 2009; 12:161-169). Riboswitches are usually found in the 5′ untranslated regions (UTRs) of bacterial mRNAs and often control gene expression in cis either at the level of transcription or translation, although other regulatory mechanisms are also known (Roth et al. The structural and functional diversity of metabolite-binding riboswitches Annu Rev Biochem 2009; 78:305-334). In most cases, metabolite binding triggers a structural rearrangement that affects the formation of either a terminator stem or a base-paired element that occludes the ribosome binding site. In addition, there is a known example of a trans-acting riboswitch (Loh et al. A trans-acting riboswitch controls expression of the virulence regulator PrfA in listeria monocytogenes. Cell 2009; 139:770-779) as well as eukaryotic riboswitches (Wachter A. Riboswitch-mediated control of gene expression in eukaryotes. RNA Biol 2010; 7:67-76) that modulate expression by controlling alternative mRNA spicing in algae (Croft et al. Thiamine biosynthesis in algae is regulated by riboswitches. Proc Natl Acad Sci USA 2007; 104:20770-20775), plants (Wachter et al. Riboswitch control of gene expression in plants by splicing and alternative 3′ end processing of mRNAs. Plant Cell 2007; 19:3437-3450), and fungi (Cheah et al. Control of alternative RNA splicing and gene expression by eukaryotic riboswitches. Nature 2007; 447:497-500).

Comparative sequence analysis methods have been developed for novel riboswitch class discovery (Rodionov et al. Regulation of lysine biosynthesis and transport genes in bacteria: yet another RNA riboswitch? Nucleic Acids Res 2003; 31:6748-6757; Barrick et al. New RNA motifs suggest an expanded scope for riboswitches in bacterial genetic control. Proc Natl Acad Sci USA 2004; 101:6421-6426; Weinberg et al., 2007). These techniques involve computational searches through genomic and metagenomic databases for sequences that are conserved both in their primary and secondary structures (Yao et al. A computational pipeline for high-throughput discovery of cis-regulatory noncoding RNA in prokaryotes. PLoS Comput Biol 2007; 3:e126; Tseng et al. Finding non-coding RNAs through genome-scale clustering. J Bioinform Comput Biol 2009; 7:373-388). Through one of these searches, the glnA motif and the Downstream-peptide motif (FIG. 29) were discovered in cyanobacteria and marine metagenomic sequences (Weinberg et al., Genome Biol 2010; 11:R31).

Structured noncoding RNAs perform many functions that are essential for protein synthesis, RNA processing, and gene regulation. Structured RNAs can be detected by comparative genomics, in which homologous sequences are identified and inspected for mutations that conserve RNA secondary structure. By applying a comparative genomics-based approach to genome and metagenome sequences from bacteria and archaea, 104 structured RNA motifs were identified. Three metabolite-binding RNA motifs were validated, including one that binds the coenzyme S-adenosylmethionine, and a further nine metabolite-binding RNA motifs were identified. New-found cis-regulatory RNA motifs are implicated in photosynthesis or nitrogen regulation in cyanobacteria, purine and one-carbon metabolism, stomach infection by Helicobacter, and many other physiological processes. A riboswitch termed crcB is represented in both bacteria and archaea. Another RNA motif controls gene expression from 3′ untranslated regions (UTRs) of mRNAs, which is unusual for bacteria. Many noncoding RNAs that act in trans are also revealed, and several of the noncoding RNA motifs are found mostly or exclusively in metagenome DNA sequences. This work greatly expands the variety of highly-structured noncoding RNAs known to exist in bacteria and archaea.

Following the discovery of complex riboswitch arrangements (Mandal et al. Science 306, 275 (2004); Welz and Breaker, RNA 13, 573 (2007); Sudarsan, et al. Science 13, 300 (2006); and E. Poiata, et al. RNA 15, 2046 (2009)) and examples of riboswitches that control alternative splicing in eukaryotes (Cheah et al. Nature 447, 497 (2007); Wachter et al. Plant Cell 19, 3437 (2007); Bocobza, et al. Genes Dev. 21, 2874 (2007); Croft et al. Proc. Natl. Acad. Sci. USA 104, 20770 (2007)) it was discovered that some of the many thousands of known group I or group II self-splicing ribozymes participate with other RNA motifs to purposefully regulate gene expression. The findings disclosed herein demonstrate that an allosteric ribozyme architecture exists naturally, and that an all-RNA component network (riboswitch, c-di-GMP, ribozyme, GTP) can be used to perform complex sensory and enzymatic functions.

It was realized that some organisms can productively harness group I ribozymes by thinking about the observation that bacteriophage ribozyme splicing is diminished in a bacterial host when ribosome function is inhibited (Sandergren and Sjoberg, J. Bacteriol. 189, 980-990 (2007)). Also, it was realized that the genomic locations and gene associations of some group I ribozymes are conserved in evolutionarily distant species (Nielsen and Johansen, RNA Biol. 6, 375 (2009)) strongly implies useful rather than purely selfish functions. Interestingly, of ten group I ribozymes present in C. difficile 630, nine are associated with a transposase gene (Table 6 in Example 1), which facilitates mobility of selfish genetic elements. Only the allosteric ribozyme described herein lacks a transposase gene, which led to the realization that this representative is not a selfish RNA element, but has a location and a function that benefits the host.

RNA engineering methods exist to couple aptamer and ribozyme domains to create allosteric ribozymes (Breaker, Curr. Opin. Biotechnol. 13, 31 (2002); Silverman, RNA 9, 377 (2003)). Interestingly, these methods were previously used to generate an allosteric group I ribozyme construct that controls gene expression when theophylline is added to cell culture (Thompson et al. BCM Biotechnol. 2, 21 (2002)). In these engineered allosteric ribozymes, theophylline aptamers were grafted to internal stems (P5 and P6) such that the presence of ligand would stabilize these important substructures of the ribozyme. In contrast, the natural allosteric group I ribozyme positions the c-di-GMP aptamer to influence folding most directly at the 5′ SS, and indirectly near the 3′ SS. However, the structural complexity of large ribozymes provides numerous additional locations wherein aptamer function can allosterically control catalysis.

Bacteria naturally exploit tandem riboswitch architectures to create more complex gene control elements (Mandal et al. Science 306, 275, 2004; Welz et al. RNA 13, 573, 2007; Sudarsan et al., Science 314, 300, 2006; Poiata et al. RNA 15, 2046, 2009). These findings add to this complexity by demonstrating how two ligand responsive RNAs, a self-splicing ribozyme and a riboswitch aptamer, collaborate to function as an allosteric RNA requiring two RNA compounds (GTP and c-di-GMP) to promote splicing. This conjoined riboswitch-ribozyme system validates a prediction made more than 20 years ago (Shub et al. Cold Spring Harb. Symp. Quant. Biol. 52, 193, 1987) that some group I ribozymes could be controlled by nucleotide-derived alarmones. Also, this RNA is a natural mimic of an engineered ribozyme that controls splicing and gene expression in response to theophylline binding (Thompson et al. BMC Biotechnol., 2, 21, 2002).

Some group I ribozymes can independently function as riboswitches that sense guanosine or one of its phosphorylated derivatives, because sufficient levels of the attacking nucleophile must be present for efficient splicing (Ames and Breaker, In: The Chemical Biology of Nucleic Acids, G. Meyer, ed., Wiley-VCH; Breaker, In: The RNA World, 4^{th} ed., Gesteland, Cech, Atkins, eds., Cold Spring Harbor Laboratory Press). The tandem riboswitch-ribozyme examined in this study could constitute a two-input gene control system that naturally reads the concentration of GTP and c-di-GMP, and trigger splicing accordingly. It is interesting to note that GTP is used as the immediate precursor for the synthesis of c-di-GMP (Hengge, Nat. Rev. Microbiol. 7, 263 (2009)), and overproduction of this second messenger causes substantial decreases in GTP concentrations in vivo (Simm et al. Mol. Microbiol. 53, 1123 (2004)). The biological utility of this tandem riboswitch-ribozyme may be used to ensure expression of the associated gene only when both compounds are present in sufficient quantities.

As the number of riboswitch discoveries expands, there are increasing opportunities to identify rare combinations of RNA structures that arrange aptamers in tandem or with other functional domains such as ribozymes. The allosteric self-splicing system described herein is one representative of many sophisticated RNA devices that are used by cells to carry out specialized sensory and catalytic functions.

### A. General Organization of Riboswitch RNAs

Bacterial riboswitch RNAs are genetic control elements that are located primarily within the 5′-untranslated region (5′-UTR) of the main coding region of a particular mRNA. Structural probing studies (discussed further below) reveal that riboswitch elements are generally composed of two domains: a natural aptamer (T. Hermann, D. J. Patel, Science 2000, 287, 820; L. Gold, et al., Annual Review of Biochemistry 1995, 64, 763) that serves as the ligand-binding domain, and an ‘expression platform’ that interfaces with RNA elements that are involved in gene expression (e.g. Shine-Dalgarno (SD) elements; transcription terminator stems). These conclusions are drawn from the observation that aptamer domains synthesized in vitro bind the appropriate ligand in the absence of the expression platform (see Examples 2, 3 and 6 of U.S. Application Publication No. 2005-0053951). Moreover, structural probing investigations indicate that the aptamer domain of most riboswitches adopts a particular secondary- and tertiary-structure fold when examined independently, that is essentially identical to the aptamer structure when examined in the context of the entire 5′ leader RNA. This indicates that, in many cases, the aptamer domain is a modular unit that folds independently of the expression platform (see Examples 2, 3 and 6 of U.S. Application Publication No. 2005-0053951).

Ultimately, the ligand-bound or unbound status of the aptamer domain is interpreted through the expression platform, which is responsible for exerting an influence upon gene expression. The view of a riboswitch as a modular element is further supported by the fact that aptamer domains are highly conserved amongst various organisms (and even between kingdoms as is observed for the TPP riboswitch), (N. Sudarsan, et al., RNA 2003, 9, 644) whereas the expression platform varies in sequence, structure, and in the mechanism by which expression of the appended open reading frame is controlled. For example, ligand binding to the TPP riboswitch of the tenA mRNA of B. subtilis causes transcription termination (A. S. Mironov, et al., Cell 2002, 111, 747). This expression platform is distinct in sequence and structure compared to the expression platform of the TPP riboswitch in the thiM mRNA from E. coli, wherein TPP binding causes inhibition of translation by a SD blocking mechanism (see Example 2 of U.S. Application Publication No. 2005-0053951). The TPP aptamer domain is easily recognizable and of near identical functional character between these two transcriptional units, but the genetic control mechanisms and the expression platforms that carry them out are very different.

Aptamer domains for riboswitch RNAs typically range from ˜70 to 170 nt in length (FIG. 11 of U.S. Application Publication No. 2005-0053951). This observation was somewhat unexpected given that in vitro evolution experiments identified a wide variety of small molecule-binding aptamers, which are considerably shorter in length and structural intricacy (T. Hermann, D. J. Patel, Science 2000, 287, 820; L. Gold, et al., Annual Review of Biochemistry 1995, 64, 763; M. Famulok, Current Opinion in Structural Biology 1999, 9, 324). Although the reasons for the substantial increase in complexity and information content of the natural aptamer sequences relative to artificial aptamers remains to be proven, this complexity is believed required to form RNA receptors that function with high affinity and selectivity. Apparent K_{D} values for the ligand-riboswitch complexes range from low nanomolar to low micromolar. It is also worth noting that some aptamer domains, when isolated from the appended expression platform, exhibit improved affinity for the target ligand over that of the intact riboswitch. (˜10 to 100-fold) (see Example 2 of U.S. Application Publication No. 2005-0053951). Presumably, there is an energetic cost in sampling the multiple distinct RNA conformations required by a fully intact riboswitch RNA, which is reflected by a loss in ligand affinity. Since the aptamer domain must serve as a molecular switch, this might also add to the functional demands on natural aptamers that might help rationalize their more sophisticated structures.

Riboswitches must be capable of discriminating against compounds related to their natural ligands to prevent undesirable regulation of metabolic genes. However, it is possible to generate analogs that trigger riboswitch function and inhibit bacterial growth, as has been demonstrated for riboswitches that normally respond to lysine (Sudarsan 2003) and thiamine pyrophosphate (Sudarsan 2006).

### B. Riboswitch Regulation of Gene Expression

Riboswitches control expression, effect, and function of RNA molecules in a variety of ways. For example, riboswitches can regulate transcription (or full transcription) of RNA molecules by, for example, causing premature termination of transcription, such as by forming a terminator signal in the RNA molecule or altering the coordination of transcription and translation of the RNA molecule. Riboswithes can also regulate tranlslation of RNA molecules by, for example, blocking or affecting binding of translation enzymes, proteins, or factors and/or altering the coordination of transcription and translation of the RNA molecule. Riboswitches can also affect expression of RNA molecules by altering processing of the RNA molecule. For example, riboswitches can modulate cleavage of the RNA molecule, splicing of the RNA molecules, latering stability of the RNA molecules (through regulation of addition or effect of RNA stability sequences, for example) and/or processing of the RNA molecules. Natural and engineered examples of such regulation by riboswitches are known and can be adapted for use with the disclosed riboswitches.

Bacteria primarily make use of two methods for termination of transcription. Certain genes incorporate a termination signal that is dependent upon the Rho protein, (J. P. Richardson, Biochimica et Biophysica Acta 2002, 1577, 251). while others make use of Rho-independent terminators (intrinsic terminators) to destabilize the transcription elongation complex (I. Gusarov, E. Nudler, Molecular Cell 1999, 3, 495; E. Nudler, M. E. Gottesman, Genes to Cells 2002, 7, 755). The latter RNA elements are composed of a GC-rich stem-loop followed by a stretch of 6-9 uridyl residues. Intrinsic terminators are widespread throughout bacterial genomes (F. Lillo, et al., 2002, 18, 971), and are typically located at the 3′-termini of genes or operons. Interestingly, an increasing number of examples are being observed for intrinsic terminators located within 5′-UTRs.

Among the wide variety of genetic regulatory strategies employed by bacteria there is a growing class of examples wherein RNA polymerase responds to a termination signal within the 5′-UTR in a regulated fashion (T. M. Henkin, Current Opinion in Microbiology 2000, 3, 149). During certain conditions the RNA polymerase complex is directed by external signals either to perceive or to ignore the termination signal. Although transcription initiation might occur without regulation, control over mRNA synthesis (and of gene expression) is ultimately dictated by regulation of the intrinsic terminator. Presumably, one of at least two mutually exclusive mRNA conformations results in the formation or disruption of the RNA structure that signals transcription termination. A trans-acting factor, which in some instances is a RNA (F. J. Grundy, et al., Proceedings of the National Academy of Sciences of the United States of America 2002, 99, 11121; T. M. Henkin, C. Yanofsky, Bioessays 2002, 24, 700) and in others is a protein (J. Stulke, Archives of Microbiology 2002, 177, 433), is generally required for receiving a particular intracellular signal and subsequently stabilizing one of the RNA conformations. Riboswitches offer a direct link between RNA structure modulation and the metabolite signals that are interpreted by the genetic control machinery.

Riboswitches can affect or regulate expression of RNA molecules by affecting processing of the RNA molecules. For example, regulation of splicing can affect processing of an RNA in which splicing is regulated. For example, a self-spicing ribozyme regulated by a riboswitch (the combination can be referred to as a riboswitch ribozyme) can be used to regulate formation of a functional (or non-functional) RNA through self-splicing.

As another example, an intron in the RNA can include an RNA processing signal or site. Splicing of the RNA can result in elimination of the processing signal or site. For example, a transcription termination signal or RNA cleavage site in the 3′ UTR of a mRNA can be deleted from the RNA if it resides in an intron that is spliced out of the RNA. Regulation of the splicing of that intron by a riboswitch as described herein can thus affect the processing of the RNA. As another example, an RNA processing signal or site can be created via splicing of an intron or different elements of an RNA processing system, signal or site can be brought into or taken out of an operable arrangement by splicing of an intron. As another example, an RNA processing signal or site can be brought into or taken out of an operable proximity with other elements of the RNA.

RNA processing can also be affected directly by a riboswitch without mediation by regulation of splicing. For example, an RNA processing signal or site can be in the expression platform domain of a riboswitch. In this way, the alteration in the structural relationship of the expression platform (and thus of the RNA processing signal or site) by activation of the riboswitch can affect processing by affecting the ability of the RNA processing signal or site to operate.

The riboswitch can affect RNA processing. By “affect RNA processing” is meant that the riboswitch can either directly or indirectly (via regulation of splicing, for example) act upon RNA to allow, stimulate, reduce or prevent RNA processing to take place. This can include, for example, allowing any processing to take place. This can increase or decrease processing fully or partially to any degree compared to the number of processing events that would have taken place without the riboswitch.

RNA processing can include, for example, transcription termination, formation of the 3′ terminus of the RNA, polyadenylation, and degradation or turnover of the RNA. As used herein, and RNA processing signal or site is a sequence, structure or location in an RNA that mediates, signals or is required for an RNA processing event or condition. For example, certain sequences or structures can signal transcription termination, RNA cleavage or polyadenylation.

The riboswitch can activate or repress splicing. By “activate splicing” is meant that the riboswitch can either directly or indirectly act upon RNA to allow splicing to take place. This can include, for example, allowing any splicing to take place (such as a single splice versus no splice) or allowing alternative splicing to take place. This can increase splicing fully or partially to any degree compared to the number of splicing events that would have taken place without the riboswitch.

By “repress splicing” is meant that the riboswitch can either directly or indirectly act upon RNA to suppress splicing. This can include, for example, preventing any splicing or reducing splicing from taking place (such as no splice versus a single splice) or preventing or reducing alternative splicing from taking place. This can decrease alternative splicing fully or partially to any degree compared to the number of alternative splicing events that would have taken place without the riboswitch.

The riboswitch can activate or repress alternative splicing. By “activate alternative splicing” is meant that the riboswitch can either directly or indirectly act upon RNA to allow alternative splicing to take place. This can increase alternative splicing fully or partially to any degree compared to the number of alternative splicing events that would have taken place without the riboswitch.

By “repress alternative splicing” is meant that the riboswitch can either directly or indirectly act upon RNA to suppress alternative splicing. This can decrease alternative splicing fully or partially to any degree compared to the number of alternative splicing events that would have taken place without the riboswitch.

The riboswitch can affect expression of a protein encoded by the RNA. For example, regulation of splicing or alternative splicing can affect the ability of the RNA to be translated, alter the coding region, or alter the translation initiation or termination. Alternative splicing can, for example, cause a start or stop codon (or both) to appear in the processed transcript that is not present in normally processed transcripts. As another example, alternative splicing can cause the normal start or stop codon to be removed from the processed transcript. A useful mode for using riboswitch-regulated splicing to regulate expression of a protein encoded by an RNA is to introduce a riboswitch in an intron in the 5′ untranslated region of the RNA and include or make use of a start codon in the intron such that the start codon in the intron will be the first start codon in the alternatively spliced RNA. Another useful mode for using riboswitch-regulated splicing to regulate expression of a protein encoded by an RNA is to introduce a riboswitch in an intron in the 5′ untranslated region of the RNA and include or make use of a short open reading frame in the intron such that the reading frame will appear first in the alternatively spliced RNA.

The RNA molecule can have a branched structure. For example, in the fungal TPP riboswitch (Cheah 2007), when TPP concentration is low, the newly transcribed mRNA adopts a structure that occludes the second 5′ splice site, while leaving the branch site available for splicing. Pre-mRNA splicing from the first 5′ splice site leads to production of the 1-3 form of mRNA and expression of the NMT1 protein. When TPP concentration is high, ligand binding to the TPP aptamer causes allosteric changes in RNA folding to increase the structural flexibility near the second 5′ splice site and to occlude nucleotides near the branch site.

Translation of RNA molecules can be regulated by riboswitches in a variety of ways. For example, a functionally significant sequence in an RNA molecule can be blocked or made accessible through action of a riboswitch. For example, the sequence of the aptamer and control strands of an aptamer domain can be adapted so that the control strand is complementary to a functionally significant sequence in an expression platform. For example, the control strand can be adapted to be complementary to the Shine-Dalgarno sequence of an RNA such that, upon formation of a stem structure between the control strand and the SD sequence, the SD sequence becomes inaccessible to ribosomes, thus reducing or preventing translation initiation. An example of this for of regulation where activation of a riboswitch causes inhibition of translation by a SD blocking mechanism is described in Example 2 of U.S. Application Publication No. 2005-0053951. As another example, the control strand can be adapted to be complementary to the initiation codon (or the region of the initiation codon) of an RNA such that, upon formation of a stem structure between the control strand and the initiation codon, the initiation codon becomes inaccessible to ribosomes, thus reducing or preventing translation initiation. As another example, the control strand can be adapted to be complementary to the binding site of a translation factor such that, upon formation of a stem structure between the control strand and the binding site, the binding site becomes inaccessible to the translation factor, thus reducing or preventing translation initiation.

### C. Features of and Methods of Using Cyclic Di-GMP-Responsive Riboswitches

Disclosed are methods and compositions for altering gene expression of genes by affecting cyclic di-GMP-responsive riboswitches operably linked to the genes, where the riboswitch comprises a cyclic di-GMP-II motif. For example, the methods can comprise bringing into contact a compound and a cell, where the compound affects the riboswitch. The cell can comprise a gene encoding an RNA comprising a cyclic di-GMP-responsive riboswitch. The riboswitch can comprise a cyclic di-GMP-II motif.

In some forms, the cell can have been identified as being in need of altered gene expression. In some forms, the cell can be a bacterial cell. In some forms, the cell can be a Clostridium, Deinococcus, or Bacillus cell. In some forms, the compound kills or inhibits the growth of the bacterial cell. In some forms, the compound and the cell can be brought into contact by administering the compound to a subject. In some forms, the cell is a bacterial cell in the subject and the compound kills or inhibits the growth of the bacterial cell. In some forms, the subject has a bacterial infection. In some forms, the compound can be administered in combination with another antimicrobial compound. In some forms, the compound inhibits bacterial growth in a biofilm.

Also disclosed are regulatable gene expression constructs comprising cyclic di-GMP-responsive riboswitches operably linked to coding regions, where the riboswitch comprises a cyclic di-GMP-II motif. For example, the disclosed constructs can comprise a nucleic acid molecule encoding an RNA comprising a riboswitch operably linked to a coding region, where the riboswitch regulates expression of the RNA, where the riboswitch and coding region are heterologous, where the riboswitch is a cyclic di-GMP-responsive riboswitch, and where the riboswitch comprises a cyclic di-GMP-II motif. As used herein, a riboswitch and coding region can be said to be heterologous if they are not operably linked in nature. For example, riboswitches and coding regions from different sources, such as different genes, different chromosomes, different organisms, and the like, can be said to be heterologous. Also disclosed are riboswitches where the riboswitch is a non-natural derivative of a naturally-occurring a cyclic di-GMP-responsive riboswitch, where the naturally-occurring riboswitch comprises a cyclic di-GMP-II motif. Also disclosed are riboswitch ribozymes comprising a riboswitch aptamer domain operably linked to a self-splicing ribozyme, where the aptamer is comprised of the cyclic di-GMP-II motif.

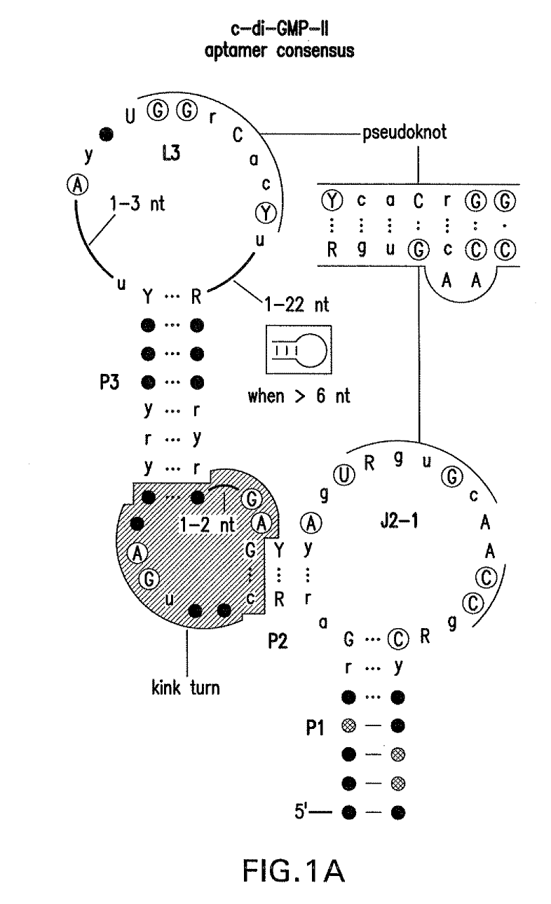

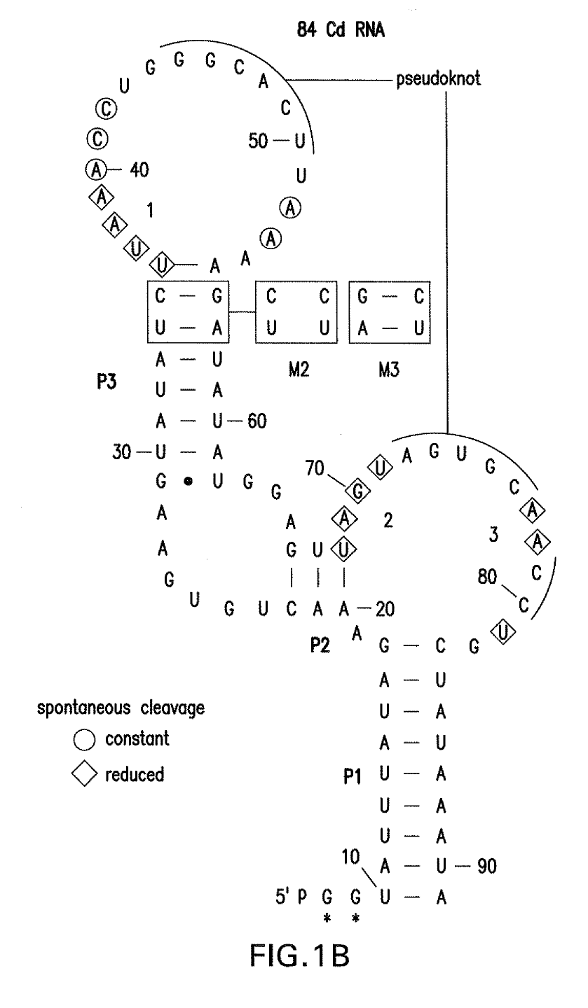

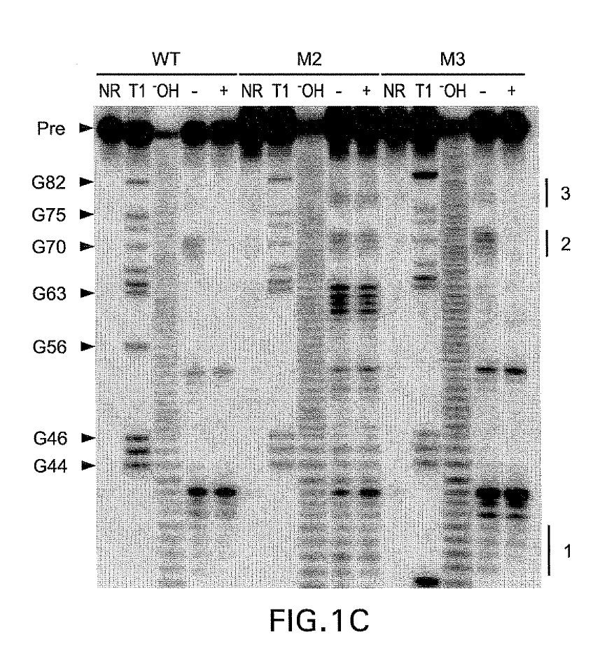

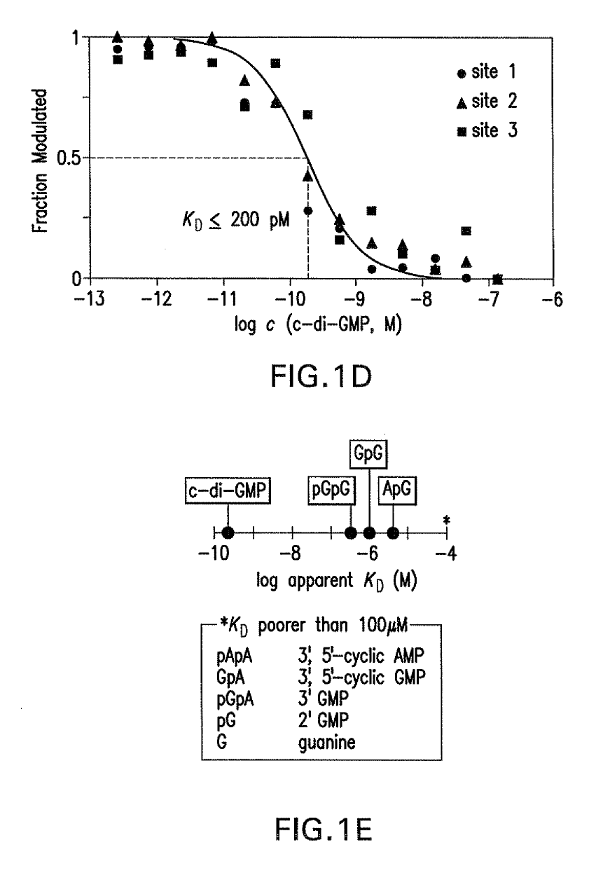

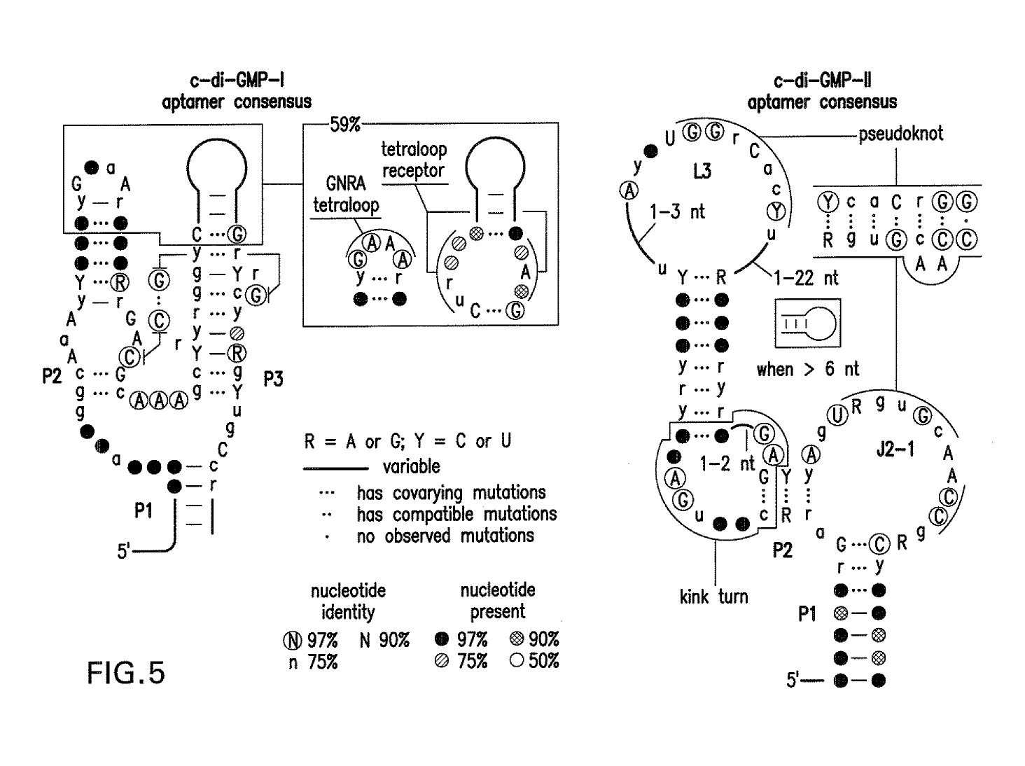

In some forms, the riboswitch can comprise an aptamer domain and an expression platform domain, where the aptamer domain and the expression platform domain are heterologous, where the aptamer is comprised of the cyclic di-GMP-II motif. In some forms, the riboswitch can comprise two or more aptamer domains and an expression platform domain, where at least one of the aptamer domains and the expression platform domain are heterologous, where at least one of the aptamer domains is comprised of the cyclic di-GMP-II motif. In some forms, at least two of the aptamer domains can exhibit cooperative binding. In some forms, the riboswitch can comprise the consensus structure of FIG. 1A or 5. As used herein, an aptamer domain and an expression platform domain can be said to be heterologous if they are not operably linked in nature. For example, aptamer domains and expression platform domains from different sources, such as different riboswitches, different genes, different chromosomes, different organisms, and the like, can be said to be heterologous.

In some forms, the riboswitch can comprise an aptamer domain and an expression platform domain, where the aptamer domain is derived from a naturally-occurring cyclic di-GMP-responsive riboswitch. In some forms, the aptamer domain can be the aptamer domain of a naturally-occurring cyclic di-GMP-responsive riboswitch. In some forms, the aptamer domain can have the consensus structure of an aptamer domain of the naturally-occurring riboswitch. In some forms, the aptamer domain can consist of only base pair conservative changes of the naturally-occurring riboswitch.

In some forms, the aptamer domain can comprise a P1 stem, where the P1 stem comprises an aptamer strand and a control strand, where the expression platform domain comprises a regulated strand, and where the regulated strand, the control strand, or both have been designed to form a stem structure. In some forms, the aptamer domain can comprise a control stem, where the control stem comprises an aptamer strand and a control strand, where the expression platform domain comprises a regulated strand, and where the regulated strand, the control strand, or both have been designed to form a stem structure.

In some forms, the riboswitch can comprise an aptamer domain and an expression platform domain, where the aptamer domain and the expression platform domain are heterologous, and where the aptamer is comprised of the cyclic di-GMP-II motif. In some forms, the riboswitch can be activated by a trigger molecule, where the riboswitch produces a signal when activated by the trigger molecule.

In some forms, the aptamer domain can comprise a control stem, where the control stem comprises an aptamer strand and a control strand, where the ribozyme comprises a regulated strand, and where the regulated strand, the control strand, or both have been designed to form a stem structure. In some forms, the aptamer domain and the ribozyme can be heterologous. In some forms, the riboswitch ribozyme can be operatively linked to a coding region, where the riboswitch ribozyme and the coding region are heterologous. As used herein, a riboswitch ribozyme and coding region can be said to be heterologous if they are not operably linked in nature. For example, riboswitch ribozymes and coding regions from different sources, such as different genes, different chromosomes, different organisms, and the like, can be said to be heterologous.

Also disclosed are methods and compositions for detecting a compound of interest. The method can comprise bringing into contact a sample and a riboswitch, where the riboswitch produces a signal when the sample contains the compound of interest. The riboswitch can be activated by the compound of interest and the riboswitch produces a signal when activated by the compound of interest. The riboswitch is a cyclic di-GMP-responsive riboswitch, where the riboswitch comprises a cyclic di-GMP-II motif.

In some forms, the riboswitch can change conformation when activated by the compound of interest, where the change in conformation produces a signal via a conformation dependent label. In some forms, the riboswitch can change conformation when activated by the compound of interest, where the change in conformation causes a change in expression of an RNA linked to the riboswitch, and where the change in expression produces a signal. In some forms, the signal can be produced by a reporter protein expressed from the RNA linked to the riboswitch.

Also disclosed are methods comprising (a) testing a compound for altering gene expression of a gene encoding an RNA comprising a riboswitch, and (b) altering gene expression by bringing into contact a cell and a compound that altered gene expression in step (a). The alteration can be via the riboswitch. The riboswitch is a cyclic di-GMP-responsive riboswitch, where the riboswitch comprises a cyclic di-GMP-II motif. The cell can comprise a gene encoding an RNA comprising a riboswitch, where the compound inhibits expression of the gene by binding to the riboswitch.

Also disclosed are methods and compositions for identifying riboswitches. The method can comprise assessing in-line spontaneous cleavage of an RNA molecule in the presence and absence of a compound, where the RNA molecule is encoded by a gene regulated by the compound, where a change in the pattern of in-line spontaneous cleavage of the RNA molecule indicates a riboswitch, where the RNA comprises a cyclic di-GMP-responsive riboswitch or a derivative of a cyclic di-GMP-responsive riboswitch. The riboswitch can comprise a cyclic di-GMP-II motif and the compound can be cyclic di-GMP.

Further disclosed are methods of killing or inhibiting the growth of bacteria. The method can comprise, for example, contacting the bacteria with a compound identified and/or confirmed by any of the methods disclosed herein. Further disclosed are methods of killing bacteria. The method can comprise, for example, contacting the bacteria with a compound identified and/or confirmed by any of the methods disclosed herein. The disclosed methods can be performed in a variety of ways and using different options or combinations of features and components. As an example, a gel-based assay or a chip-based assay can be used to determine if the test compound interacts with, modulates, inhibits, blocks, deactivates, and/or activates the riboswitch, such as a cyclic diGMP riboswitch. The test compound can interact in any manner, such as, for example, via van der Waals interactions, hydrogen bonds, electrostatic interactions, hydrophobic interactions, or a combination. The riboswitch, such as a cyclic diGMP riboswitch, can comprise an RNA cleaving ribozyme, for example. A fluorescent signal can be generated when a nucleic acid comprising a quenching moiety is cleaved. Molecular beacon technology can be employed to generate the fluorescent signal. The methods disclosed herein can be carried out using a high throughput screen.

Also disclosed are compositions and methods for selecting and identifying compounds that can activate, deactivate or block a riboswitch, such as a cyclic diGMP riboswitch. Activation of a riboswitch, such as a cyclic di-GMP riboswitch, refers to the change in state of the riboswitch upon binding of a trigger molecule. A riboswitch, such as a cyclic di-GMP riboswitch, can be activated by compounds other than the trigger molecule and in ways other than binding of a trigger molecule. The term trigger molecule is used herein to refer to molecules and compounds that can activate a riboswitch. This includes the natural or normal trigger molecule for the riboswitch and other compounds that can activate the riboswitch. Natural or normal trigger molecules are the trigger molecule for a given riboswitch in nature or, in the case of some non-natural riboswitches, the trigger molecule for which the riboswitch was designed or with which the riboswitch was selected (as in, for example, in vitro selection or in vitro evolution techniques). Non-natural trigger molecules can be referred to as non-natural trigger molecules.

Deactivation of a riboswitch refers to the change in state of the riboswitch, such as a cyclic di-GMP riboswitch, when the trigger molecule is not bound. A riboswitch, such as a cyclic di-GMP riboswitch, can be deactivated by binding of compounds other than the trigger molecule and in ways other than removal of the trigger molecule. Blocking of a riboswitch, such as a cyclic di-GMP riboswitch, refers to a condition or state of the riboswitch where the presence of the trigger molecule does not activate the riboswitch. Activation of a riboswitch, such as a cyclic di-GMP riboswitch, can be assessed in any suitable manner. For example, the riboswitch, such as a cyclic di-GMP riboswitch, can be linked to a reporter RNA and expression, expression level, or change in expression level of the reporter RNA can be measured in the presence and absence of the test compound. As another example, the riboswitch, such as a cyclic di-GMP riboswitch, can include a conformation dependent label, the signal from which changes depending on the activation state of the riboswitch, such as a cyclic di-GMP riboswitch. Such a riboswitch preferably uses an aptamer domain from or derived from a naturally occurring riboswitch. As can be seen, assessment of activation of a riboswitch can be performed with the use of a control assay or measurement or without the use of a control assay or measurement. Methods for identifying compounds that deactivate a riboswitch can be performed in analogous ways.

Also disclosed are method of inhibiting growth of a cell, such as a bacterial cell, that is in a subject. The method can comprise administering to the subject an effective amount of a compound identified and/or confirmed in any of the methods described herein. This can result in the compound being brought into contact with the cell. The subject can have, for example, a bacterial infection, and the bacterial cells can be the cells to be inhibited by the compound. The bacteria can be any bacteria, such as bacteria from the genus Clostridium, Deinococcus, or Bacillus, for example. Bacterial growth can also be inhibited in any context in which bacteria are found. For example, bacterial growth in fluids, biofilms, and on surfaces can be inhibited. The compounds disclosed herein can be administered or used in combination with any other compound or composition. For example, the disclosed compounds can be administered or used in combination with another antimicrobial compound.

It is to be understood that the disclosed methods and compositions are not limited to specific examples unless otherwise specified, and, as such, can vary. It is also to be understood that the terminology used herein is for the purpose of describing particular embodiments only and is not intended to be limiting.

### Materials

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference to each of various individual and collective combinations and permutation of these compounds can not be explicitly disclosed, each is specifically contemplated and described herein. For example, if a riboswitch or aptamer domain is disclosed and discussed and a number of modifications that can be made to a number of molecules including the riboswitch or aptamer domain are discussed, each and every combination and permutation of riboswitch or aptamer domain and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C—F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Riboswitches

Riboswitches are expression control elements that are part of an RNA molecule to be expressed and that change state when bound by a trigger molecule. Riboswitches typically can be dissected into two separate domains: one that selectively binds the target (aptamer domain) and another that influences genetic control (expression platform domain). It is the dynamic interplay between these two domains that results in metabolite-dependent allosteric control of gene expression. Disclosed are isolated and recombinant riboswitches, recombinant constructs containing such riboswitches, heterologous sequences operably linked to such riboswitches, and cells and transgenic organisms harboring such riboswitches, riboswitch recombinant constructs, and riboswitches operably linked to heterologous sequences. The heterologous sequences can be, for example, sequences encoding proteins or peptides of interest, including reporter proteins or peptides. Preferred riboswitches are, or are derived from, naturally occurring riboswitches. As used herein, a riboswitch and a sequence can be said to be heterologous if they are not operably linked in nature. For example, riboswitches and sequences from different sources, such as different genes, different chromosomes, different organisms, and the like, can be said to be heterologous.

The disclosed riboswitches, including the derivatives and recombinant forms thereof, generally can be from any source, including naturally occurring riboswitches and riboswitches designed de novo. Any such riboswitches can be used in or with the disclosed methods. However, different types of riboswitches can be defined and some such sub-types can be useful in or with particular methods (generally as described elsewhere herein). Types of riboswitches include, for example, naturally occurring riboswitches, derivatives and modified forms of naturally occurring riboswitches, chimeric riboswitches, and recombinant riboswitches. A naturally occurring riboswitch is a riboswitch having the sequence of a riboswitch as found in nature. Such a naturally occurring riboswitch can be an isolated or recombinant form of the naturally occurring riboswitch as it occurs in nature. That is, the riboswitch has the same primary structure but has been isolated or engineered in a new genetic or nucleic acid context. Chimeric riboswitches can be made up of, for example, part of a riboswitch of any or of a particular class or type of riboswitch and part of a different riboswitch of the same or of any different class or type of riboswitch; part of a riboswitch of any or of a particular class or type of riboswitch and any non-riboswitch sequence or component. Recombinant riboswitches are riboswitches that have been isolated or engineered in a new genetic or nucleic acid context.

Riboswitches can have single or multiple aptamer domains. Aptamer domains in riboswitches having multiple aptamer domains can exhibit cooperative binding of trigger molecules or can not exhibit cooperative binding of trigger molecules (that is, the aptamers need not exhibit cooperative binding). In the latter case, the aptamer domains can be said to be independent binders. Riboswitches having multiple aptamers can have one or multiple expression platform domains. For example, a riboswitch having two aptamer domains that exhibit cooperative binding of their trigger molecules can be linked to a single expression platform domain that is regulated by both aptamer domains. Riboswitches having multiple aptamers can have one or more of the aptamers joined via a linker. Where such aptamers exhibit cooperative binding of trigger molecules, the linker can be a cooperative linker.

Aptamer domains can be said to exhibit cooperative binding if they have a Hill coefficient n between x and x−1, where x is the number of aptamer domains (or the number of binding sites on the aptamer domains) that are being analyzed for cooperative binding. Thus, for example, a riboswitch having two aptamer domains (such as glycine-responsive riboswitches) can be said to exhibit cooperative binding if the riboswitch has Hill coefficient between 2 and 1. It should be understood that the value of x used depends on the number of aptamer domains being analyzed for cooperative binding, not necessarily the number of aptamer domains present in the riboswitch. This makes sense because a riboswitch can have multiple aptamer domains where only some exhibit cooperative binding.

Disclosed are chimeric riboswitches containing heterologous aptamer domains and expression platform domains. That is, chimeric riboswitches are made up an aptamer domain from one source and an expression platform domain from another source. The heterologous sources can be from, for example, different specific riboswitches, different types of riboswitches, or different classes of riboswitches. The heterologous aptamers can also come from non-riboswitch aptamers. The heterologous expression platform domains can also come from non-riboswitch sources.

Modified or derivative riboswitches can be produced using in vitro selection and evolution techniques. In general, in vitro evolution techniques as applied to riboswitches involve producing a set of variant riboswitches where part(s) of the riboswitch sequence is varied while other parts of the riboswitch are held constant. Activation, deactivation or blocking (or other functional or structural criteria) of the set of variant riboswitches can then be assessed and those variant riboswitches meeting the criteria of interest are selected for use or further rounds of evolution. Useful base riboswitches for generation of variants are the specific and consensus riboswitches disclosed herein. Consensus riboswitches can be used to inform which part(s) of a riboswitch to vary for in vitro selection and evolution.

Also disclosed are modified riboswitches with altered regulation. The regulation of a riboswitch can be altered by operably linking an aptamer domain to the expression platform domain of the riboswitch (which is a chimeric riboswitch). The aptamer domain can then mediate regulation of the riboswitch through the action of, for example, a trigger molecule for the aptamer domain. Aptamer domains can be operably linked to expression platform domains of riboswitches in any suitable manner, including, for example, by replacing the normal or natural aptamer domain of the riboswitch with the new aptamer domain. Generally, any compound or condition that can activate, deactivate or block the riboswitch from which the aptamer domain is derived can be used to activate, deactivate or block the chimeric riboswitch.

Also disclosed are inactivated riboswitches. Riboswitches can be inactivated by covalently altering the riboswitch (by, for example, crosslinking parts of the riboswitch or coupling a compound to the riboswitch). Inactivation of a riboswitch in this manner can result from, for example, an alteration that prevents the trigger molecule for the riboswitch from binding, that prevents the change in state of the riboswitch upon binding of the trigger molecule, or that prevents the expression platform domain of the riboswitch from affecting expression upon binding of the trigger molecule.

Also disclosed are biosensor riboswitches. Biosensor riboswitches are engineered riboswitches that produce a detectable signal in the presence of their cognate trigger molecule. Useful biosensor riboswitches can be triggered at or above threshold levels of the trigger molecules. Biosensor riboswitches can be designed for use in vivo or in vitro. For example, biosensor riboswitches operably linked to a reporter RNA that encodes a protein that serves as or is involved in producing a signal can be used in vivo by engineering a cell or organism to harbor a nucleic acid construct encoding the riboswitch/reporter RNA. An example of a biosensor riboswitch for use in vitro is a riboswitch that includes a conformation dependent label, the signal from which changes depending on the activation state of the riboswitch. Such a biosensor riboswitch preferably uses an aptamer domain from or derived from a naturally occurring riboswitch. Biosensor riboswitches can be used in various situations and platforms. For example, biosensor riboswitches can be used with solid supports, such as plates, chips, strips and wells.

Also disclosed are modified or derivative riboswitches that recognize new trigger molecules. New riboswitches and/or new aptamers that recognize new trigger molecules can be selected for, designed or derived from known riboswitches. This can be accomplished by, for example, producing a set of aptamer variants in a riboswitch, assessing the activation of the variant riboswitches in the presence of a compound of interest, selecting variant riboswitches that were activated (or, for example, the riboswitches that were the most highly or the most selectively activated), and repeating these steps until a variant riboswitch of a desired activity, specificity, combination of activity and specificity, or other combination of properties results.

In general, any aptamer domain can be adapted for use with any expression platform domain by designing or adapting a regulated strand in the expression platform domain to be complementary to the control strand of the aptamer domain. Alternatively, the sequence of the aptamer and control strands of an aptamer domain can be adapted so that the control strand is complementary to a functionally significant sequence in an expression platform. For example, the control strand can be adapted to be complementary to the Shine-Dalgarno sequence of an RNA such that, upon formation of a stem structure between the control strand and the SD sequence, the SD sequence becomes inaccessible to ribosomes, thus reducing or preventing translation initiation. Note that the aptamer strand would have corresponding changes in sequence to allow formation of a P1 stem in the aptamer domain. In the case of riboswitches having multiple aptamers exhibiting cooperative binding, one the P1 stem of the activating aptamer (the aptamer that interacts with the expression platform domain) need be designed to form a stem structure with the SD sequence.

As used herein, a control stem is a stem structure that can form in an aptamer domain of a riboswitch, where the control stem is formed from an aptamer strand and a control strand. As used herein, a regulated stem is a stem structure that can form in a riboswitch, where the regulated stem is formed from a control strand and a regulated strand. Control strands are part of aptamer domains of riboswitches that can form a stem structure with the aptamer strand of the riboswitch. Aptamer strands are part of aptamer domains of riboswitches that can form a stem structure with the control strand of the riboswitch. Regulated strands are part of expression platform domains of riboswitches that can form a stem structure with the control strand of the riboswitch. Thus, the control strand can form alternative stem structures in a riboswitch, generally based on whether the riboswitch is bound by a trigger molecule or not. One of the alternative stem structures is the control stem, where the control strand forms a stem with the aptamer strand. The other alternative stem structure is the regulated stem, where the control strand forms a stem with the regulated strand. Control stems, control strands, and aptamer strands can be referred to as being of or belonging to or being comprised in a riboswitch or aptamer domain. Regulated strands can be referred to as being of or belonging to or being comprised in a riboswitch or expression platform domain. Regulated stems can be referred to as being of or belonging to or being comprised in a riboswitch. Because regulated stems are comprised of one strand of the aptamer domain and one strand of the expression platform domain, regulated stems can also be referred to as being of or belonging to or being comprised in the aptamer domain or the expression platform domain.

In some forms, a transcription terminator can be added to an RNA molecule (most conveniently in an untranslated region of the RNA) where part of the sequence of the transcription terminator is complementary to the control strand of an aptamer domain (the sequence will be the regulated strand). This will allow the control strand of the aptamer domain to form alternative stem structures with the aptamer strand and the regulated strand, thus either forming or disrupting a transcription terminator stem upon activation or deactivation of the riboswitch. Any other expression element can be brought under the control of a riboswitch by similar design of alternative stem structures.

For transcription terminators controlled by riboswitches, the speed of transcription and spacing of the riboswitch and expression platform elements can be important for proper control. Transcription speed can be adjusted by, for example, including polymerase pausing elements (e.g., a series of uridine residues) to pause transcription and allow the riboswitch to form and sense trigger molecules.

Disclosed are regulatable gene expression constructs comprising a nucleic acid molecule encoding an RNA comprising a riboswitch operably linked to a coding region, where the riboswitch regulates expression of the RNA, where the riboswitch and coding region are heterologous, where the riboswitch is a cyclic di-GMP-responsive riboswitch, and where the riboswitch comprises a cyclic di-GMP-II motif. The riboswitch can comprise an aptamer domain and an expression platform domain, where the aptamer domain and the expression platform domain are heterologous. The riboswitch can comprise an aptamer domain and an expression platform domain, where the aptamer domain comprises a P1 stem, where the P1 stem comprises an aptamer strand and a control strand, where the expression platform domain comprises a regulated strand, where the regulated strand, the control strand, or both have been designed to form a stem structure. The riboswitch can comprise two or more aptamer domains and an expression platform domain, where at least one of the aptamer domains and the expression platform domain are heterologous. The riboswitch can comprise two or more aptamer domains and an expression platform domain, where at least one of the aptamer domains comprises a P1 stem, where the P1 stem comprises an aptamer strand and a control strand, where the expression platform domain comprises a regulated strand, where the regulated strand, the control strand, or both have been designed to form a stem structure.

Riboswitches can be referred to in different ways. For example, riboswitches can be identified by their trigger molecule (or main or natural trigger molecule): cyclic di-GMP riboswitch or SAM/SAH riboswitch, for example. Riboswitches can be identified by their responsiveness to a trigger molecule: cyclic di-GMP-responsive riboswitch or SAH-responsive riboswitch, for example. Riboswitches can be identified by the aptamer in the riboswitch: cyclic di-GMP-II, Downstream-peptide riboswitch, or crcB riboswitch, for example. Examples of riboswitches include cyclic di-GMP riboswitches and cyclic di-GMP-II riboswitches.

1. Aptamer Domains

Aptamers are nucleic acid segments and structures that can bind selectively to particular compounds and classes of compounds. Riboswitches have aptamer domains that, upon binding of a trigger molecule result in a change in the state or structure of the riboswitch. In functional riboswitches, the state or structure of the expression platform domain linked to the aptamer domain changes when the trigger molecule binds to the aptamer domain. Aptamer domains of riboswitches can be derived from any source, including, for example, natural aptamer domains of riboswitches, artificial aptamers, engineered, selected, evolved or derived aptamers or aptamer domains. Aptamers in riboswitches generally have at least one portion that can interact, such as by forming a stem structure, with a portion of the linked expression platform domain. This stem structure will either form or be disrupted upon binding of the trigger molecule. In the disclosed riboswitches, the aptamer domains can be or can be derived from cyclic di-GMP riboswitches and cyclic di-GMP-II riboswitches and can contain to be comprised of cyclic di-GMP-II mtifs.

Consensus and specific aptamer domains of cyclic di-GMP riboswitches and cyclic di-GMP-II riboswitches a variety of natural riboswitches are shown in FIGS. 1A, 1B, 2A, 2C, 3A, 3D, 5, and 8A and elsewhere herein. These aptamer domains (including all of the direct variants embodied therein) can be used in riboswitches. The consensus sequences and structures indicate variations in sequence and structure. Aptamer domains that are within the indicated variations are referred to herein as direct variants. These aptamer domains can be modified to produce modified or variant aptamer domains. Conservative modifications include any change in base paired nucleotides such that the nucleotides in the pair remain complementary. Moderate modifications include changes in the length of stems or of loops (for which a length or length range is indicated) of less than or equal to 20% of the length range indicated. Loop and stem lengths are considered to be “indicated” where the consensus structure shows a stem or loop of a particular length or where a range of lengths is listed or depicted. Moderate modifications include changes in the length of stems or of loops (for which a length or length range is not indicated) of less than or equal to 40% of the length range indicated. Moderate modifications also include and functional variants of unspecified portions of the aptamer domain. Consensus aptamer domains of a variety of other natural riboswitches are shown in FIG. 11 of U.S. Application Publication No. 2005-0053951. These aptamer domains (including all of the direct variants embodied therein) can be used in riboswitches.

The P1 stem (or control stem) and its constituent strands can be modified in adapting aptamer domains for use with expression platforms and RNA molecules. Such modifications, which can be extensive, are referred to herein as P1 modifications or control stem modifications. P1 modifications include changes to the sequence and/or length of the P1 stem of an aptamer domain.

Aptamer domains of the disclosed riboswitches can also be used for any other purpose, and in any other context, as aptamers. For example, aptamers can be used to control ribozymes, other molecular switches, and any RNA molecule where a change in structure can affect function of the RNA.

2. Expression Platform Domains

Expression platform domains are a part of riboswitches that affect expression of the RNA molecule that contains the riboswitch. Expression platform domains generally have at least one portion that can interact, such as by forming a stem structure, with a portion of the linked aptamer domain. This stem structure, which can be referred to as a regulated stem, will either form or be disrupted upon binding of the trigger molecule. The stem structure generally either is, or prevents formation of, or prevents access to, an expression regulatory structure. An expression regulatory structure is a structure that allows, prevents, enhances or inhibits expression of an RNA molecule containing the structure. Examples include Shine-Dalgarno sequences, initiation codons, expression factor binding sites, transcription terminators, splicing signals, stability signals, and processing signals.

### B. Trigger Molecules

Trigger molecules are molecules and compounds that can activate a riboswitch. This includes the natural or normal trigger molecule for the riboswitch and other compounds that can activate the riboswitch. Natural or normal trigger molecules are the trigger molecule for a given riboswitch in nature or, in the case of some non-natural riboswitches, the trigger molecule for which the riboswitch was designed or with which the riboswitch was selected (as in, for example, in vitro selection or in vitro evolution techniques). For the disclosed riboswitches, preferred trigger molecules are or are structurally related to cyclic diGMP.

C. Cyclic Di-GMP Riboswitches (Cyclic diGMP-II Motifs)

Disclosed is a new riboswitch motif class, termed cyclic di-GMP-II motif class. The cyclic di-GMP-II motif forms two internal bulges using three base-paired regions (P1, P2 and P3), and an imperfect pseudoknot structure between nucleotides of the P3 loop (L3) and the junction linking stems P2 and P1 (J2-1) (FIG. 1A). The internal bulge between P2 and P3 conforms to a kink-turn motif (Klein et al. EMBO J. 20, 4214 (2001); Winkler et al. RNA 7, 1165 (2001)). Although, 24 of the 28 bacterial species that carry this riboswitch also carry c-di-GMP-I riboswitches, it was realized that representatives of the new RNA motif serve as aptamers for a distinct class of c-di-GMP riboswitches, termed c-di-GMP-II.

A representative RNA from C. difficile was subjected to in-line probing (Soukup and Breaker, RNA 5, 1308 (1999); E. E. Regulski, R. R. Breaker, Methods Mol. Biol. 419, 53 (2008)). An 84-nucleotide 5′ ³²P-labeled RNA, corresponding to the conserved motif located upstream of a putative virulence gene in C. difficile (FIG. 1B), was incubated in the absence or presence of 10 μM c-di-GMP under in-line probing conditions. Spontaneous RNA cleavage products were separated by denaturing polyacrylamide gel electrophoresis (PAGE), and band locations and intensities (FIG. 1C) were used to map regions of structural change. The observed pattern of cleavage products produced in the absence of c-di-GMP is consistent with the structural model generated by comparative sequence analysis. Furthermore, 11 internucleotide linkages in four spans within the L3 and J2-1 regions exhibit reduced strand scission when c-di-GMP is present (FIGS. 1B and 1C), which indicates that the RNA structure is stabilized by binding the second messenger.

In-line probing reactions using a range of c-di-GMP concentrations resulted in a binding curve with an apparent dissociation constant (K_{D}) of ˜200 μM (FIG. 1D). The concentrations of c-di-GMP range from a minimum of 260 fM to a maximum of 1.2 μM, with increments of 3-fold increases in concentration. This apparent K_{D} approaches the limit of sensitivity of the assay (Regulski and Breaker, Methods Mol. Biol. 419, 53 (2008)), and therefore the true affinity may be even better. Similarly, the affinities were determined for various analogs of c-di-GMP, revealing that the RNA discriminates against the linear form of the second messenger and other analogs by more than three orders of magnitude (FIG. 1E). The molecular recognition characteristics displayed by this RNA are similar to those observed for c-di-GMP-I aptamers (Sudarsan et al. Science 321, 411 (2008); Smith et al. Nat. Struct. Mol. Biol. 16, 1218 (2009)), and therefore it is concluded that the RNA represents a new class of aptamers for this second messenger termed c-di-GMP-II.

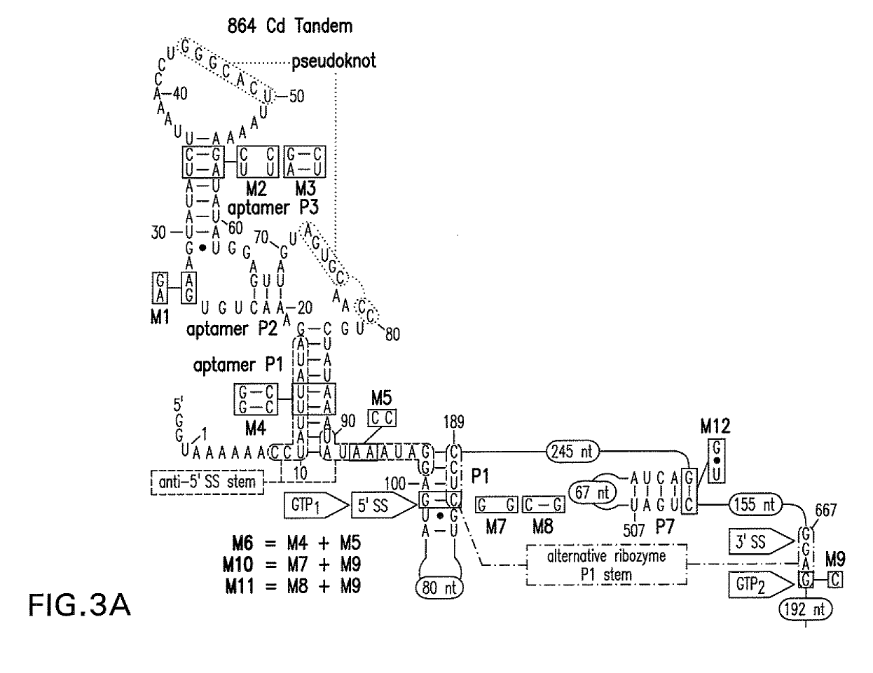

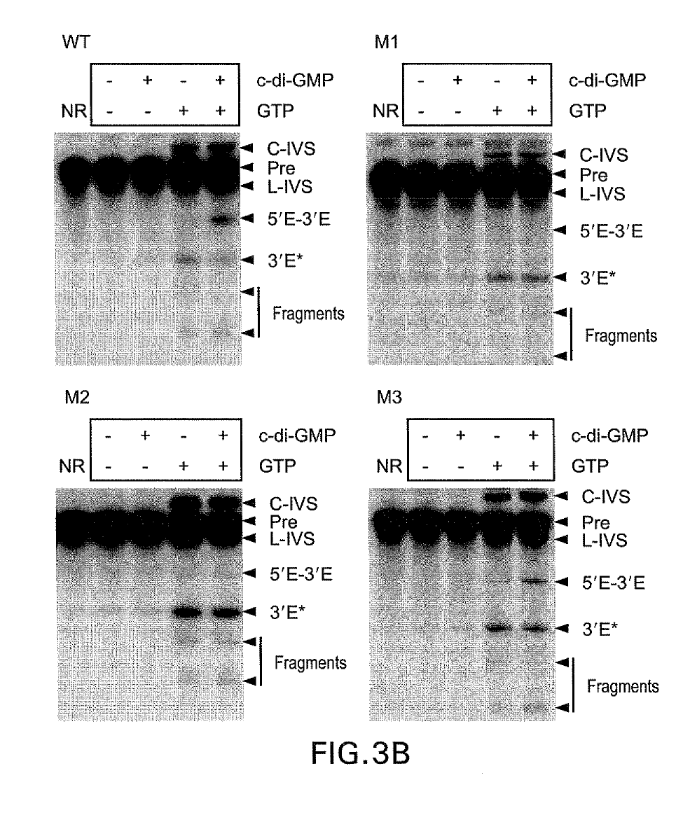

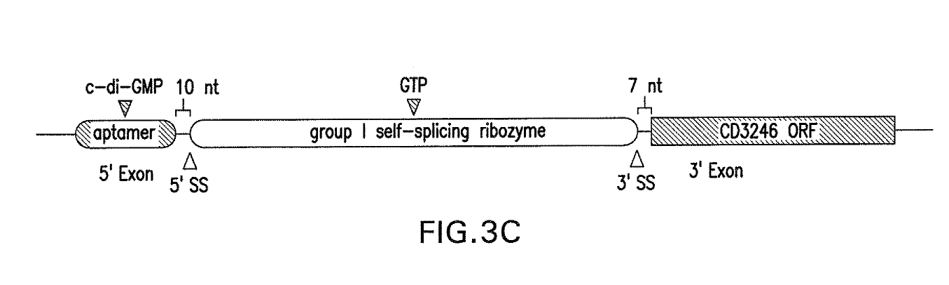

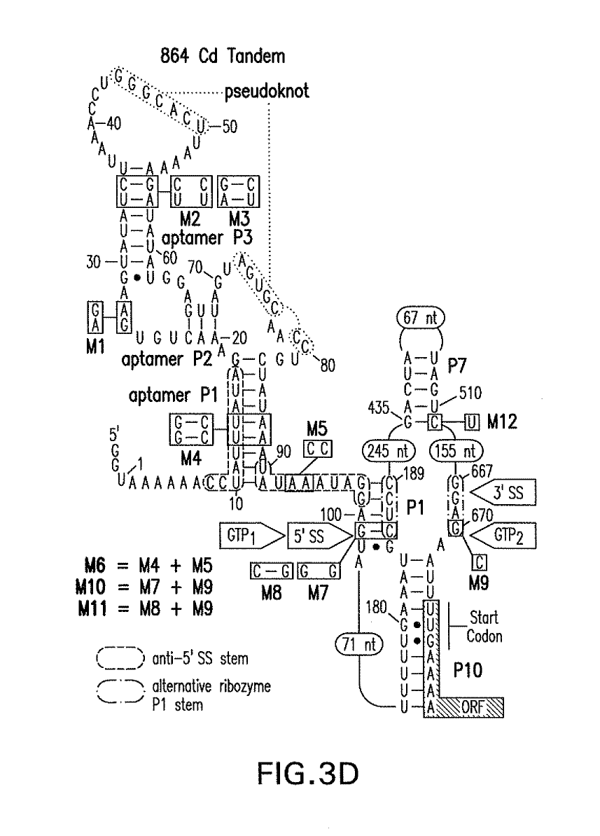

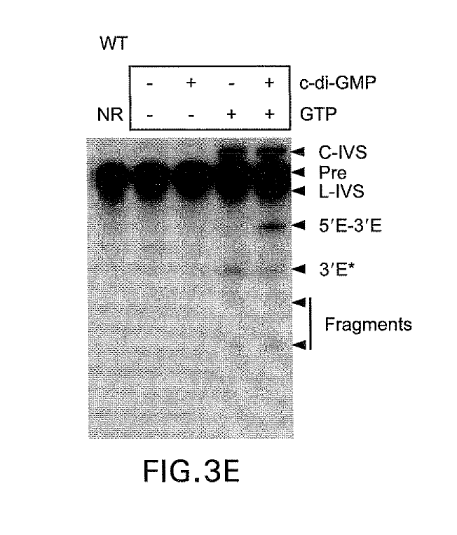

The example c-di-GMP-II riboswitch studied here is operably linked to and regulates activity of a self-splicing ribozyme. Two alternative base pairing structures were noticed that could explain how c-di-GMP binding controls splicing in the studied example of c-di-GMP-II riboswitch. The first alternative stem, called anti-5′ SS (FIG. 3A, enclosed in dased line oval), includes the left shoulder of the aptamer P1 (nucleotides 8 through 17), which are complementary to nucleotides (90 through 99) that link the aptamer and ribozyme domains. The second alternative stem, called alternative ribozyme P1 (FIG. 3A, enclosed in alternating dash dot oval), includes the right shoulder of ribozyme P1 (nucleotides 186 through 189), which are complementary to nucleotides (667 through 670) near the 3′ end of the ribozyme.

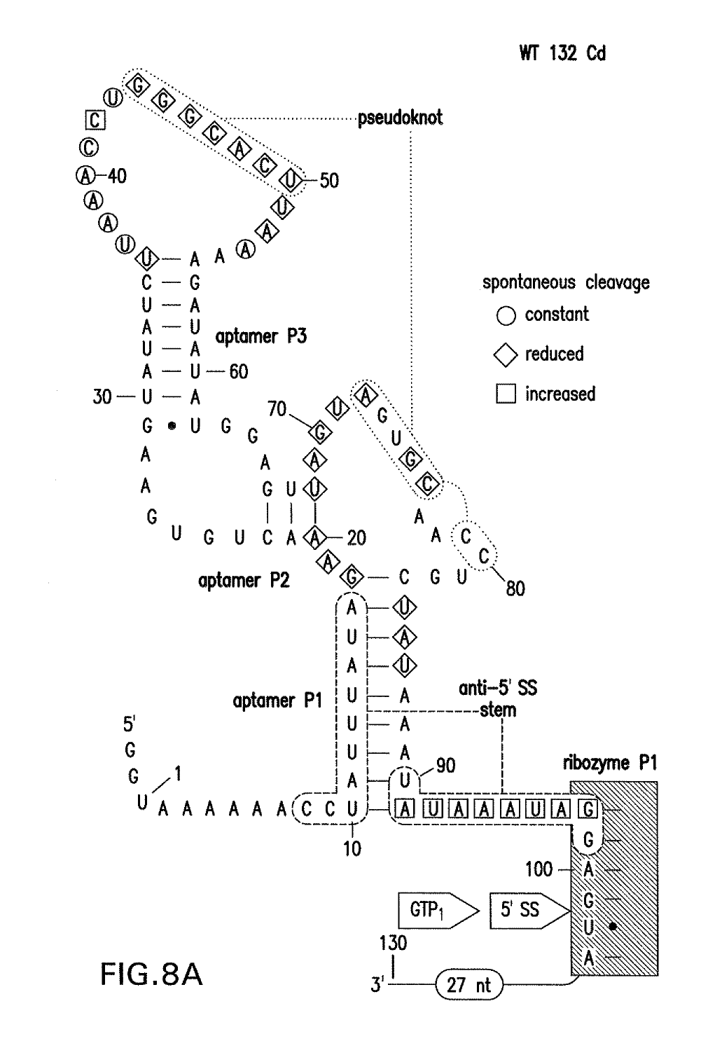

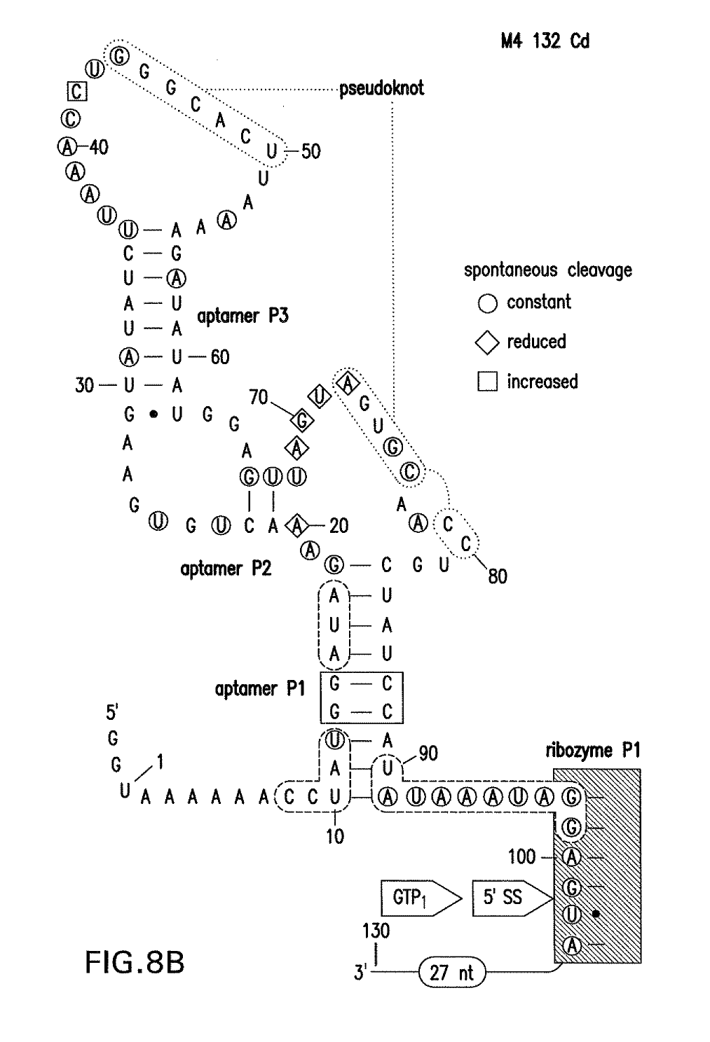

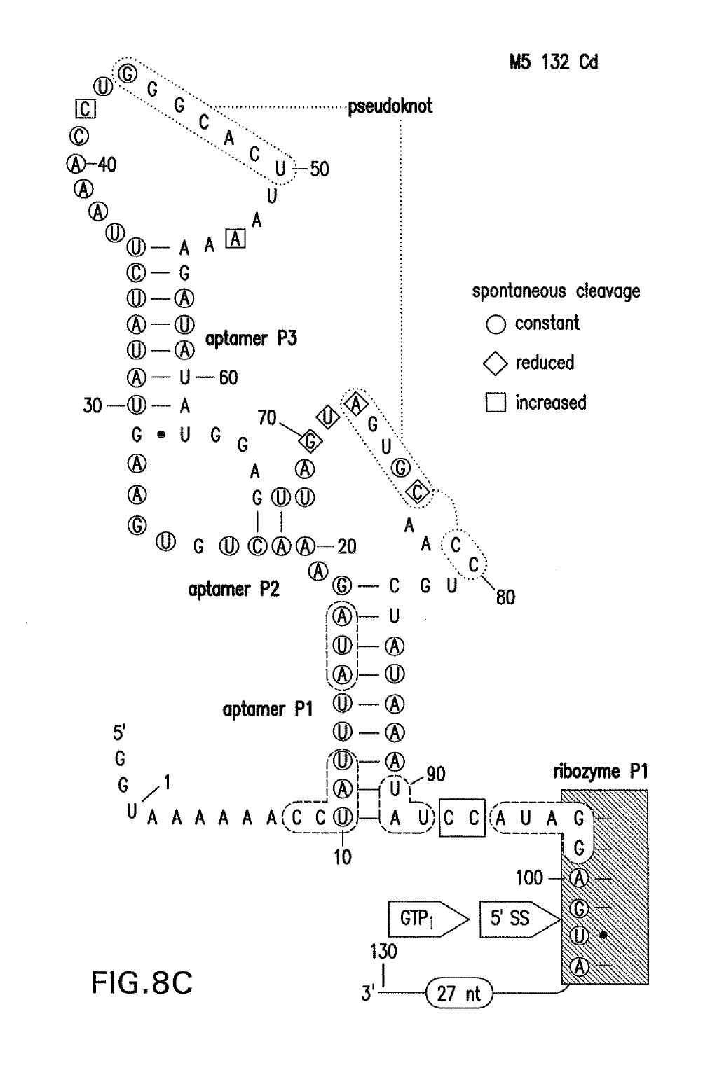

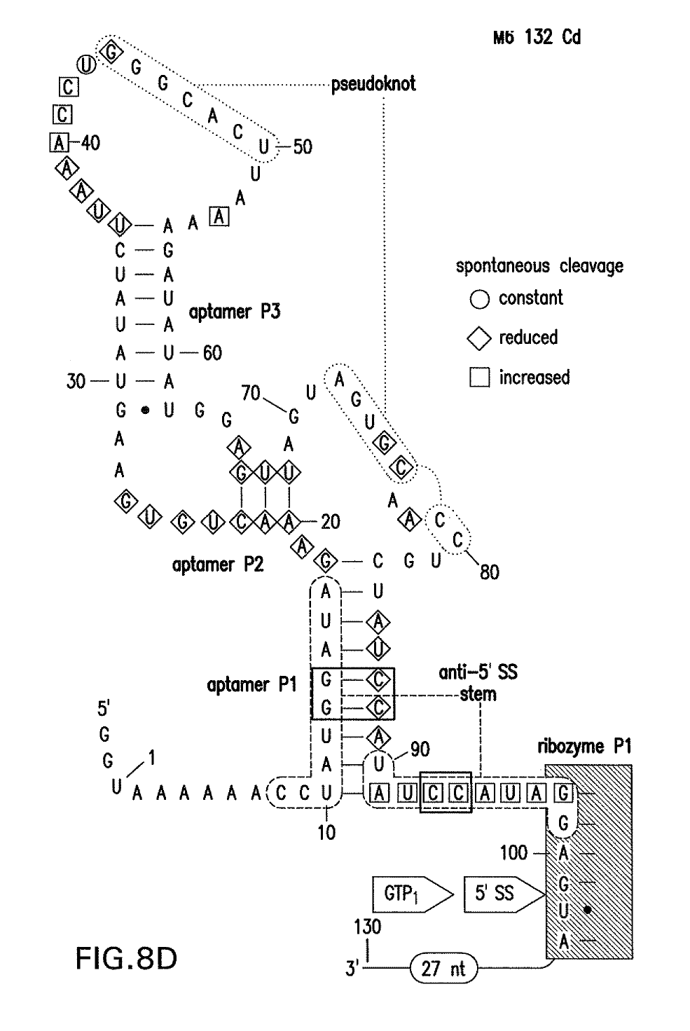

Formation of the anti-5′ SS stem disrupts two base pairs of the ribozyme P1 stem. Since c-di-GMP is expected to stabilize the proposed aptamer structure, the absence of c-di-GMP will weaken the aptamer P1 stem, and favor formation of the anti-5′ SS stem. Therefore, low concentrations of c-di-GMP can inhibit formation of the stem carrying the 5′ SS and prevent GTP attack at the 5′ SS. As predicted, c-di-GMP addition yields a spontaneous cleavage pattern consistent with aptamer P1 stem formation, while the absence of the second messenger permits the RNA to form the anti-5′ SS alternative pairing (FIG. 8A). The changes in the pattern of in-line probing data upon addition of c-di-GMP are consistent with formation of the alternative base-paired structure in the absence of ligand. Concentrations of c-di-GMP range from a minimum of 260 fM to a maximum of 1.2 μM, with increments of 9-fold increases in concentration.

Mutants M1 through M3 were prepared to examine the role of c-di-GMP binding on ribozyme splicing. Mutants M4 through M11 were prepared to assess other structures proposed to be important for allosteric ribozyme function, and mutant 12 was prepared to examine this group I ribozyme's usage of the typical guanosine binding pocket. The effects on self-splicing regulation of mutant M4, which retains aptamer P1 formation but disrupts the first alternative pairing, were also examined. M4 retains c-di-GMP binding activity (FIG. 8B), but splicing is no longer responsive to the second messenger, indicating that the ability to form the anti-5′ SS stem is necessary for allosteric control of ribozyme activity. Moreover, although M4 is no longer responsive to c-di-GMP, the yield of spliced exons is higher in the absence of the second messenger comparted to WT, which is expected if the aptamer P1 stem is not effectively competing with anti-5′ SS stem formation.

Construct M5, which carries mutations in the linker between the aptamer and the ribozyme, was expected to have similar activity as M4. This mutant loses splicing control as expected, but the RNA is defective in ligand binding and does not produce fully spliced products. Although the latter results are unexpected, in-line probing reveals that this mutant RNA is misfolded (FIG. 8C), which accounts for its lack of function. In contrast, placing M4 and M5 mutations in the same construct (M6) restores the potential for anti-5′ SS stem formation (FIG. 8D), and likewise restores allosteric activity of the construct. Collectively, the characteristics of these mutants strongly support the allosteric control mechanism involving mutually exclusive formation of aptamer P1 and anti-5′ SS stems. Similar experiments using additional mutants yield results that also support the formation of the alternative ribozyme P1 stem.

### D. Self-Splicing Ribozymes

Ribozymes can act on other RNA molecules or can act upon themselves. A self-splicing ribozyme is a ribonucleic acid which is defined by its capability to excise itself from a transcript in which it is located. The presence of this self-splicing ability is a preferable feature of the methods of the invention because this removes the need for additional components (e.g. the spliceosome) for a successful splicing reaction to occur. Such ribozymes are observed as introns in transcribed nucleic acids in a wide range of organisms, including prokaryotic and eukaryotic species, and also in viruses (e.g. Ko et al., 2002, J. Bact. 184: 3917-3922 Bonocora and Shub, 2004, J. Bact. 86: 8153-8155; Yamada et al., 1994 Nucleic Acids Research 22: 2532-2537).

In the transcribed nucleic acid, the functional structure of the ribozyme forms. The ribozyme then proceeds, through a series of reactions, to catalyse the cleavage and rejoining of the transcript such that the ribozyme is excised. Self-splicing ribozymes can in theory be incorporated anywhere within an encoding nucleic acid, but will only be removed from a transcribed region. Ribozymes only fold into their active form in any single-stranded RNA. The methods of this invention are equally applicable to the mutagenesis of messenger RNA (mRNA), transfer RNA (tRNA) and ribosomal RNA (rRNA). Use of self-splicing ribozymes is particularly advantageous as it permits the insertion of a nucleic acid fragment into a target nucleic acid, which will be excised when the sequence is present in a single-stranded ribonucleic acid, for example where the sequence to be removed is incorporated into the ribozyme.

### E. Constructs, Vectors and Expression Systems

The disclosed riboswitches, such as cyclic diGMP riboswitches, can be used with any suitable expression system. Recombinant expression is usefully accomplished using a vector, such as a plasmid. The vector can include a promoter operably linked to riboswitch-encoding sequence and RNA to be expression (e.g., RNA encoding a protein). The vector can also include other elements required for transcription and translation. As used herein, vector refers to any carrier containing exogenous DNA. Thus, vectors are agents that transport the exogenous nucleic acid into a cell without degradation and include a promoter yielding expression of the nucleic acid in the cells into which it is delivered. Vectors include but are not limited to plasmids, viral nucleic acids, viruses, phage nucleic acids, phages, cosmids, and artificial chromosomes. A variety of prokaryotic and eukaryotic expression vectors suitable for carrying riboswitch-regulated constructs can be produced. Such expression vectors include, for example, pET, pET3d, pCR2.1, pBAD, pUC, and yeast vectors. The vectors can be used, for example, in a variety of in vivo and in vitro situation.

Viral vectors include adenovirus, adeno-associated virus, herpes virus, vaccinia virus, polio virus, AIDS virus, neuronal trophic virus, Sindbis and other RNA viruses, including these viruses with the HIV backbone. Also useful are any viral families which share the properties of these viruses which make them suitable for use as vectors. Retroviral vectors, which are described in Verma (1985), include Murine Maloney Leukemia virus, MMLV, and retroviruses that express the desirable properties of MMLV as a vector. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral DNA.

A “promoter” is generally a sequence or sequences of DNA that function when in a relatively fixed location in regard to the transcription start site. A “promoter” contains core elements required for basic interaction of RNA polymerase and transcription factors and can contain upstream elements and response elements.

“Enhancer” generally refers to a sequence of DNA that functions at no fixed distance from the transcription start site and can be either 5′ (Laimins, 1981) or 3′ (Lusky et al., 1983) to the transcription unit. Furthermore, enhancers can be within an intron (Banerji et al., 1983) as well as within the coding sequence itself (Osborne et al., 1984). They are usually between 10 and 300 bp in length, and they function in cis. Enhancers function to increase transcription from nearby promoters. Enhancers, like promoters, also often contain response elements that mediate the regulation of transcription. Enhancers often determine the regulation of expression.

Expression vectors used in eukaryotic host cells (yeast, fungi, insect, plant, animal, human or nucleated cells) can also contain sequences necessary for the termination of transcription which can affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs.

The vector can include nucleic acid sequence encoding a marker product. This marker product is used to determine if the gene has been delivered to the cell and once delivered is being expressed. Preferred marker genes are the E. Coli lacZ gene which encodes β-galactosidase and green fluorescent protein.

In some embodiments the marker can be a selectable marker. When such selectable markers are successfully transferred into a host cell, the transformed host cell can survive if placed under selective pressure. There are two widely used distinct categories of selective regimes. The first category is based on a cell's metabolism and the use of a mutant cell line which lacks the ability to grow independent of a supplemented media. The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern and Berg, 1982), mycophenolic acid, (Mulligan and Berg, 1980) or hygromycin (Sugden et al., 1985).

Gene transfer can be obtained using direct transfer of genetic material, in but not limited to, plasmids, viral vectors, viral nucleic acids, phage nucleic acids, phages, cosmids, and artificial chromosomes, or via transfer of genetic material in cells or carriers such as cationic liposomes. Such methods are well known in the art and readily adaptable for use in the method described herein. Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)). Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991).

1. Viral Vectors

Preferred viral vectors are Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Preferred retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors have higher transaction (ability to introduce genes) abilities than do most chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

i. Retroviral Vectors

A retrovirus is an animal virus belonging to the virus family of Retroviridae, including any types, subfamilies, genus, or tropisms. Retroviral vectors, in general, are described by Verma, I. M., Retroviral vectors for gene transfer. In Microbiology-1985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

ii. Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A preferred viral vector is one based on an adenovirus which has had the E1 gene removed and these virons are generated in a cell line such as the human 293 cell line. In another preferred embodiment both the E1 and E3 genes are removed from the adenovirus genome.

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and can contain upstream elements and response elements.

2. Viral Promoters and Enhancers

Preferred promoters controlling transcription from vectors in mammalian host cells can be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell. Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell. Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, α-fetoprotein and insulin), typically one will use an enhancer from a eukaryotic cell virus. Preferred examples are the SV40 enhancer on the late side of the replication origin (bp 100-270), the cytomegalovirus early promoter enhancer, the polyoma enhancer on the late side of the replication origin, and adenovirus enhancers.

The promoter and/or enhancer can be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

It is preferred that the promoter and/or enhancer region be active in all eukaryotic cell types. A preferred promoter of this type is the CMV promoter (650 bases). Other preferred promoters are SV40 promoters, cytomegalovirus (full length promoter), and retroviral vector LTF.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells, for example) can also contain sequences necessary for the termination of transcription which can affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In a preferred embodiment of the transcription unit, the polyadenylation region is derived from the SV40 early polyadenylation signal and consists of about 400 bases. It is also preferred that the transcribed units contain other standard sequences alone or in combination with the above sequences improve expression from, or stability of, the construct.

3. Markers

The vectors can include nucleic acid sequence encoding a marker product. This marker product is used to determine if the gene has been delivered to the cell and once delivered is being expressed. Preferred marker genes are the E. Coli lacZ gene, which encodes β-galactosidase, and green fluorescent protein.

In some embodiments the marker can be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR⁻ cells and mouse LTK⁻ cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

### F. Biosensor Riboswitches

Also disclosed are biosensor riboswitches. Biosensor riboswitches are engineered riboswitches that produce a detectable signal in the presence of their cognate trigger molecule. Useful biosensor riboswitches can be triggered at or above threshold levels of the trigger molecules. Biosensor riboswitches can be designed for use in vivo or in vitro. For example, biosensor riboswitches operably linked to a reporter RNA that encodes a protein that serves as or is involved in producing a signal can be used in vivo by engineering a cell or organism to harbor a nucleic acid construct encoding the riboswitch/reporter RNA. An example of a biosensor riboswitch for use in vitro is a riboswitch that includes a conformation dependent label, the signal from which changes depending on the activation state of the riboswitch. Such a biosensor riboswitch preferably uses an aptamer domain from or derived from a naturally occurring riboswitch, such as from a cyclic diGMP riboswitch.

### G. Reporter Proteins and Peptides

For assessing activation of a riboswitch, or for biosensor riboswitches, a reporter protein or peptide can be used. The reporter protein or peptide can be encoded by the RNA the expression of which is regulated by the riboswitch. The examples describe the use of some specific reporter proteins. The use of reporter proteins and peptides is well known and can be adapted easily for use with riboswitches. The reporter proteins can be any protein or peptide that can be detected or that produces a detectable signal. Preferably, the presence of the protein or peptide can be detected using standard techniques (e.g., radioimmunoassay, radio-labeling, immunoassay, assay for enzymatic activity, absorbance, fluorescence, luminescence, and Western blot). More preferably, the level of the reporter protein is easily quantifiable using standard techniques even at low levels. Useful reporter proteins include luciferases, green fluorescent proteins and their derivatives, such as firefly luciferase (FL) from Photinus pyralis, and Renilla luciferase (RL) from Renilla reniformis.

### H. Conformation Dependent Labels

Conformation dependent labels refer to all labels that produce a change in fluorescence intensity or wavelength based on a change in the form or conformation of the molecule or compound (such as a riboswitch) with which the label is associated. Examples of conformation dependent labels used in the context of probes and primers include molecular beacons, Amplifluors, FRET probes, cleavable FRET probes, TaqMan probes, scorpion primers, fluorescent triplex oligos including but not limited to triplex molecular beacons or triplex FRET probes, fluorescent water-soluble conjugated polymers, PNA probes and QPNA probes. Such labels, and, in particular, the principles of their function, can be adapted for use with riboswitches. Several types of conformation dependent labels are reviewed in Schweitzer and Kingsmore, Curr. Opin. Biotech. 12:21-27 (2001).

Stem quenched labels, a form of conformation dependent labels, are fluorescent labels positioned on a nucleic acid such that when a stem structure forms a quenching moiety is brought into proximity such that fluorescence from the label is quenched. When the stem is disrupted (such as when a riboswitch containing the label is activated), the quenching moiety is no longer in proximity to the fluorescent label and fluorescence increases. Examples of this effect can be found in molecular beacons, fluorescent triplex oligos, triplex molecular beacons, triplex FRET probes, and QPNA probes, the operational principles of which can be adapted for use with riboswitches.

Stem activated labels, a form of conformation dependent labels, are labels or pairs of labels where fluorescence is increased or altered by formation of a stem structure. Stem activated labels can include an acceptor fluorescent label and a donor moiety such that, when the acceptor and donor are in proximity (when the nucleic acid strands containing the labels form a stem structure), fluorescence resonance energy transfer from the donor to the acceptor causes the acceptor to fluoresce. Stem activated labels are typically pairs of labels positioned on nucleic acid molecules (such as riboswitches) such that the acceptor and donor are brought into proximity when a stem structure is formed in the nucleic acid molecule. If the donor moiety of a stem activated label is itself a fluorescent label, it can release energy as fluorescence (typically at a different wavelength than the fluorescence of the acceptor) when not in proximity to an acceptor (that is, when a stem structure is not formed). When the stem structure forms, the overall effect would then be a reduction of donor fluorescence and an increase in acceptor fluorescence. FRET probes are an example of the use of stem activated labels, the operational principles of which can be adapted for use with riboswitches.

### I. Detection Labels

To aid in detection and quantitation of riboswitch activation, deactivation or blocking, or expression of nucleic acids or protein produced upon activation, deactivation or blocking of riboswitches, detection labels can be incorporated into detection probes or detection molecules or directly incorporated into expressed nucleic acids or proteins. As used herein, a detection label is any molecule that can be associated with nucleic acid or protein, directly or indirectly, and which results in a measurable, detectable signal, either directly or indirectly. Many such labels are known to those of skill in the art. Examples of detection labels suitable for use in the disclosed method are radioactive isotopes, fluorescent molecules, phosphorescent molecules, enzymes, antibodies, and ligands.

Examples of suitable fluorescent labels include fluorescein isothiocyanate (FITC), 5,6-carboxymethyl fluorescein, Texas red, nitrobenz-2-oxa-1,3-diazol-4-yl (NBD), coumarin, dansyl chloride, rhodamine, amino-methyl coumarin (AMCA), Eosin, Erythrosin, BODIPY®, Cascade Blue®, Oregon Green®, pyrene, lissamine, xanthenes, acridines, oxazines, phycoerythrin, macrocyclic chelates of lanthanide ions such as quantum Dye™, fluorescent energy transfer dyes, such as thiazole orange-ethidium heterodimer, and the cyanine dyes Cy3, Cy3.5, Cy5, Cy5.5 and Cy7. Examples of other specific fluorescent labels include 3-Hydroxypyrene 5,8,10-Tri Sulfonic acid, 5-Hydroxy Tryptamine (5-HT), Acid Fuchsin, Alizarin Complexon, Alizarin Red, Allophycocyanin, Aminocoumarin, Anthroyl Stearate, Astrazon Brilliant Red 4G, Astrazon Orange R, Astrazon Red 6B, Astrazon Yellow 7 GLL, Atabrine, Auramine, Aurophosphine, Aurophosphine G, BAO 9 (Bisaminophenyloxadiazole), BCECF, Berberine Sulphate, Bisbenzamide, Blancophor FFG Solution, Blancophor SV, Bodipy Fl, Brilliant Sulphoflavin FF, Calcien Blue, Calcium Green, Calcofluor RW Solution, Calcofluor White, Calcophor White ABT Solution, Calcophor White Standard Solution, Carbostyryl, Cascade Yellow, Catecholamine, Chinacrine, Coriphosphine O, Coumarin-Phalloidin, CY3.1 8, CY5.1 8, CY7, Dans (1-Dimethyl Amino Naphaline 5 Sulphonic Acid), Dansa (Diamino Naphtyl Sulphonic Acid), Dansyl NH—CH3, Diamino Phenyl Oxydiazole (DAO), Dimethylamino-5-Sulphonic acid, Dipyrrometheneboron Difluoride, Diphenyl Brilliant Flavine 7GFF, Dopamine, Erythrosin ITC, Euchrysin, FIF (Formaldehyde Induced Fluorescence), Flazo Orange, Fluo 3, Fluorescamine, Fura-2, Genacryl Brilliant Red B, Genacryl Brilliant Yellow 10GF, Genacryl Pink 3G, Genacryl Yellow 5GF, Gloxalic Acid, Granular Blue, Haematoporphyrin, Indo-1, Intrawhite Cf Liquid, Leucophor PAF, Leucophor SF, Leucophor WS, Lissamine Rhodamine B200 (RD200), Lucifer Yellow CH, Lucifer Yellow VS, Magdala Red, Marina Blue, Maxilon Brilliant Flavin 10 GFF, Maxilon Brilliant Flavin 8 GFF, MPS (Methyl Green Pyronine Stilbene), Mithramycin, NBD Amine, Nitrobenzoxadidole, Noradrenaline, Nuclear Fast Red, Nuclear Yellow, Nylosan Brilliant Flavin E8G, Oxadiazole, Pacific Blue, Pararosaniline (Feulgen), Phorwite AR Solution, Phorwite BKL, Phorwite Rev, Phorwite RPA, Phosphine 3R, Phthalocyanine, Phycoerythrin R, Polyazaindacene Pontochrome Blue Black, Porphyrin, Primuline, Procion Yellow, Pyronine, Pyronine B, Pyrozal Brilliant Flavin 7GF, Quinacrine Mustard, Rhodamine 123, Rhodamine 5 GLD, Rhodamine 6G, Rhodamine B, Rhodamine B 200, Rhodamine B Extra, Rhodamine BB, Rhodamine BG, Rhodamine WT, Serotonin, Sevron Brilliant Red 2B, Sevron Brilliant Red 4G, Sevron Brilliant Red B, Sevron Orange, Sevron Yellow L, SITS (Primuline), SITS (Stilbene Isothiosulphonic acid), Stilbene, Snarf 1, sulpho Rhodamine B Can C, Sulpho Rhodamine G Extra, Tetracycline, Thiazine Red R, Thioflavin S, Thioflavin TCN, Thioflavin 5, Thiolyte, Thiozol Orange, Tinopol CBS, True Blue, Ultralite, Uranine B, Uvitex SFC, Xylene Orange, and XRITC.

Useful fluorescent labels are fluorescein (5-carboxyfluorescein-N-hydroxysuccinimide ester), rhodamine (5,6-tetramethyl rhodamine), and the cyanine dyes Cy3, Cy3.5, Cy5, Cy5.5 and Cy7. The absorption and emission maxima, respectively, for these fluors are: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm), thus allowing their simultaneous detection. Other examples of fluorescein dyes include 6-carboxyfluorescein (6-FAM), 2′,4′,1,4,-tetrachlorofluorescein (TET), 2′,4′,5′,7′,1,4-hexachlorofluorescein (HEX), 2′,7′-dimethoxy-4′,5′-dichloro-6-carboxyrhodamine (JOE), 2′-chloro-5′-fluoro-7′,8′-fused phenyl-1,4-dichloro-6-carboxyfluorescein (NED), and 2′-chloro-7′-phenyl-1,4-dichloro-6-carboxyfluorescein (VIC). Fluorescent labels can be obtained from a variety of commercial sources, including Amersham Pharmacia Biotech, Piscataway, N.J.; Molecular Probes, Eugene, Oreg.; and Research Organics, Cleveland, Ohio.

Additional labels of interest include those that provide for signal only when the probe with which they are associated is specifically bound to a target molecule, where such labels include: “molecular beacons” as described in Tyagi & Kramer, Nature Biotechnology (1996) 14:303 and EP 0 070 685 B1. Other labels of interest include those described in U.S. Pat. No. 5,563,037; WO 97/17471 and WO 97/17076.

Labeled nucleotides are a useful form of detection label for direct incorporation into expressed nucleic acids during synthesis. Examples of detection labels that can be incorporated into nucleic acids include nucleotide analogs such as BrdUrd (5-bromodeoxyuridine, Hoy and Schimke, Mutation Research 290:217-230 (1993)), aminoallyldeoxyuridine (Henegariu et al., Nature Biotechnology 18:345-348 (2000)), 5-methylcytosine (Sano et al., Biochim. Biophys. Acta 951:157-165 (1988)), bromouridine (Wansick et al., J. Cell Biology 122:283-293 (1993)) and nucleotides modified with biotin (Langer et al., Proc. Natl. Acad. Sci. USA 78:6633 (1981)) or with suitable haptens such as digoxygenin (Kerkhof, Anal. Biochem. 205:359-364 (1992)). Suitable fluorescence-labeled nucleotides are Fluorescein-isothiocyanate-dUTP, Cyanine-3-dUTP and Cyanine-5-dUTP (Yu et al., Nucleic Acids Res., 22:3226-3232 (1994)). A preferred nucleotide analog detection label for DNA is BrdUrd (bromodeoxyuridine, BrdUrd, BrdU, BUdR, Sigma-Aldrich Co). Other useful nucleotide analogs for incorporation of detection label into DNA are AA-dUTP (aminoallyl-deoxyuridine triphosphate, Sigma-Aldrich Co.), and 5-methyl-dCTP (Roche Molecular Biochemicals). A useful nucleotide analog for incorporation of detection label into RNA is biotin-16-UTP (biotin-16-uridine-5′-triphosphate, Roche Molecular Biochemicals). Fluorescein, Cy3, and Cy5 can be linked to dUTP for direct labeling. Cy3.5 and Cy7 are available as avidin or anti-digoxygenin conjugates for secondary detection of biotin- or digoxygenin-labeled probes.

Detection labels that are incorporated into nucleic acid, such as biotin, can be subsequently detected using sensitive methods well-known in the art. For example, biotin can be detected using streptavidin-alkaline phosphatase conjugate (Tropix, Inc.), which is bound to the biotin and subsequently detected by chemiluminescence of suitable substrates (for example, chemiluminescent substrate CSPD: disodium, 3-(4-methoxyspiro-[1,2,-dioxetane-3-2′-(5′-chloro)tricyclo [3.3.1.1^{3,7}]decane]-4-yl) phenyl phosphate; Tropix, Inc.). Labels can also be enzymes, such as alkaline phosphatase, soybean peroxidase, horseradish peroxidase and polymerases, that can be detected, for example, with chemical signal amplification or by using a substrate to the enzyme which produces light (for example, a chemiluminescent 1,2-dioxetane substrate) or fluorescent signal.

Molecules that combine two or more of these detection labels are also considered detection labels. Any of the known detection labels can be used with the disclosed probes, tags, molecules and methods to label and detect activated or deactivated riboswitches or nucleic acid or protein produced in the disclosed methods. Methods for detecting and measuring signals generated by detection labels are also known to those of skill in the art. For example, radioactive isotopes can be detected by scintillation counting or direct visualization; fluorescent molecules can be detected with fluorescent spectrophotometers; phosphorescent molecules can be detected with a spectrophotometer or directly visualized with a camera; enzymes can be detected by detection or visualization of the product of a reaction catalyzed by the enzyme; antibodies can be detected by detecting a secondary detection label coupled to the antibody. As used herein, detection molecules are molecules which interact with a compound or composition to be detected and to which one or more detection labels are coupled.

### J. Sequence Similarities

It is understood that as discussed herein the use of the terms homology and identity mean the same thing as similarity. Thus, for example, if the use of the word homology is used between two sequences (non-natural sequences, for example) it is understood that this is not necessarily indicating an evolutionary relationship between these two sequences, but rather is looking at the similarity or relatedness between their nucleic acid sequences. Many of the methods for determining homology or identity between two evolutionarily related molecules are routinely applied to any two or more nucleic acids or proteins for the purpose of measuring sequence similarity or identity regardless of whether they are evolutionarily related or not.

In general, it is understood that one way to define any known variants and derivatives or those that might arise, of the disclosed riboswitches, aptamers, expression platforms, genes and proteins herein, is through defining the variants and derivatives in terms of homology or identity to specific known sequences. This identity of particular sequences disclosed herein is also discussed elsewhere herein. In general, variants of riboswitches, aptamers, expression platforms, genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology or identity to a stated sequence or a native sequence. Those of skill in the art readily understand how to determine the homology or identity of two proteins or nucleic acids, such as genes. For example, the homology or identity can be calculated after aligning the two sequences so that the homology or identity is at its highest level.

Another way of calculating homology or identity can be performed by published algorithms. Optimal alignment of sequences for comparison can be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology or identity can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods can differ, but the skilled artisan understands if identity is found with at least one of these methods, the sequences would be said to have the stated identity.

For example, as used herein, a sequence recited as having a particular percent homology or identity to another sequence refers to sequences that have the recited homology or identity as calculated by any one or more of the calculation methods described above. For example, a first sequence has 80 percent homology or identity, as defined herein, to a second sequence if the first sequence is calculated to have 80 percent homology or identity to the second sequence using the Zuker calculation method even if the first sequence does not have 80 percent homology or identity to the second sequence as calculated by any of the other calculation methods. As another example, a first sequence has 80 percent homology or identity, as defined herein, to a second sequence if the first sequence is calculated to have 80 percent homology or identity to the second sequence using both the Zuker calculation method and the Pearson and Lipman calculation method even if the first sequence does not have 80 percent homology or identity to the second sequence as calculated by the Smith and Waterman calculation method, the Needleman and Wunsch calculation method, the Jaeger calculation methods, or any of the other calculation methods. As yet another example, a first sequence has 80 percent homology or identity, as defined herein, to a second sequence if the first sequence is calculated to have 80 percent homology or identity to the second sequence using each of calculation methods (although, in practice, the different calculation methods will often result in different calculated homology or identity percentages).

### K. Hybridization and Selective Hybridization

The term hybridization typically means a sequence driven interaction between at least two nucleic acid molecules, such as a primer or a probe and a riboswitch or a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization can involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting nucleic acid is in for example, 10 or 100 or 1000 fold excess. This type of assay can be performed at under conditions where both the limiting and non-limiting nucleic acids are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of nucleic acid that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the nucleic acid is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the nucleic acid molecules are extended. Preferred conditions also include those suggested by the manufacturer or indicated in the art as being appropriate for the enzyme performing the manipulation.

Just as with homology and identity, it is understood that there are a variety of methods herein disclosed for determining the level of hybridization between two nucleic acid molecules. It is understood that these methods and conditions can provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

### L. Nucleic Acids

There are a variety of molecules disclosed herein that are nucleic acid based, including, for example, riboswitches, aptamers, and nucleic acids that encode riboswitches and aptamers. The disclosed nucleic acids can be made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if a nucleic acid molecule is introduced into a cell or cell environment through for example exogenous delivery, it is advantageous that the nucleic acid molecule be made up of nucleotide analogs that reduce the degradation of the nucleic acid molecule in the cellular environment.

So long as their relevant function is maintained, riboswitches, aptamers, expression platforms and any other oligonucleotides and nucleic acids can be made up of or include modified nucleotides (nucleotide analogs). Many modified nucleotides are known and can be used in oligonucleotides and nucleic acids. A nucleotide analog is a nucleotide which contains some type of modification to either the base, sugar, or phosphate moieties. Modifications to the base moiety would include natural and synthetic modifications of A, C, G, and T/U as well as different purine or pyrimidine bases, such as uracil-5-yl, hypoxanthin-9-yl (I), and 2-aminoadenin-9-yl. A modified base includes but is not limited to 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Additional base modifications can be found for example in U.S. Pat. No. 3,687,808, Englisch et al., Angewandte Chemie, International Edition, 1991, 30, 613, and Sanghvi, Y. S., Chapter 15, Antisense Research and Applications, pages 289-302, Crooke, S. T. and Lebleu, B. ed., CRC Press, 1993. Certain nucleotide analogs, such as 5-substituted pyrimidines, 6-azapyrimidines and N-2, N-6 and O-6 substituted purines, including 2-aminopropyladenine, 5-propynyluracil and 5-propynylcytosine. 5-methylcytosine can increase the stability of duplex formation. Other modified bases are those that function as universal bases. Universal bases include 3-nitropyrrole and 5-nitroindole. Universal bases substitute for the normal bases but have no bias in base pairing. That is, universal bases can base pair with any other base. Base modifications often can be combined with for example a sugar modification, such as 2′-β-methoxyethyl, to achieve unique properties such as increased duplex stability. There are numerous United States patents such as 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; and 5,681,941, which detail and describe a range of base modifications. Each of these patents is herein incorporated by reference in its entirety, and specifically for their description of base modifications, their synthesis, their use, and their incorporation into oligonucleotides and nucleic acids.

Nucleotide analogs can also include modifications of the sugar moiety. Modifications to the sugar moiety would include natural modifications of the ribose and deoxyribose as well as synthetic modifications. Sugar modifications include but are not limited to the following modifications at the 2′ position: OH; F; 0-, S—, or N-alkyl; 0-, S—, or N-alkenyl; 0-, S- or N-alkynyl; or O-alkyl-O-alkyl, wherein the alkyl, alkenyl and alkynyl can be substituted or unsubstituted C1 to C10, alkyl or C2 to C10 alkenyl and alkynyl. 2′ sugar modifications also include but are not limited to —O[(CH₂)n O]m CH₃, —O(CH₂)n OCH₃, —O(CH₂)n NH₂, —O(CH₂)n CH₃, —O(CH₂)n —ONH₂, and —O(CH₂)nON[(CH₂)n CH₃)]₂, where n and m are from 1 to about 10.

Other modifications at the 2′ position include but are not limited to: C1 to C10 lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂ CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications can also be made at other positions on the sugar, particularly the 3′ position of the sugar on the 3′ terminal nucleotide or in 2′-5′ linked oligonucleotides and the 5′ position of 5′ terminal nucleotide. Modified sugars would also include those that contain modifications at the bridging ring oxygen, such as CH₂ and S. Nucleotide sugar analogs can also have sugar mimetics such as cyclobutyl moieties in place of the pentofuranosyl sugar. There are numerous United States patents that teach the preparation of such modified sugar structures such as 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety, and specifically for their description of modified sugar structures, their synthesis, their use, and their incorporation into nucleotides, oligonucleotides and nucleic acids.

Nucleotide analogs can also be modified at the phosphate moiety. Modified phosphate moieties include but are not limited to those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. It is understood that these phosphate or modified phosphate linkages between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts and free acid forms are also included. Numerous United States patents teach how to make and use nucleotides containing modified phosphates and include but are not limited to, 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference its entirety, and specifically for their description of modified phosphates, their synthesis, their use, and their incorporation into nucleotides, oligonucleotides and nucleic acids.

It is understood that nucleotide analogs need only contain a single modification, but can also contain multiple modifications within one of the moieties or between different moieties.

Nucleotide substitutes are molecules having similar functional properties to nucleotides, but which do not contain a phosphate moiety, such as peptide nucleic acid (PNA). Nucleotide substitutes are molecules that will recognize and hybridize to (base pair to) complementary nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

Nucleotide substitutes are nucleotides or nucleotide analogs that have had the phosphate moiety and/or sugar moieties replaced. Nucleotide substitutes do not contain a standard phosphorus atom. Substitutes for the phosphate can be for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts. Numerous United States patents disclose how to make and use these types of phosphate replacements and include but are not limited to 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference its entirety, and specifically for their description of phosphate replacements, their synthesis, their use, and their incorporation into nucleotides, oligonucleotides and nucleic acids.

It is also understood in a nucleotide substitute that both the sugar and the phosphate moieties of the nucleotide can be replaced, by for example an amide type linkage (aminoethylglycine) (PNA). U.S. Pat. Nos. 5,539,082; 5,714,331; and 5,719,262 teach how to make and use PNA molecules, each of which is herein incorporated by reference (see also Nielsen et al., Science 254:1497-1500 (1991)).

Oligonucleotides and nucleic acids can be comprised of nucleotides and can be made up of different types of nucleotides or the same type of nucleotides. For example, one or more of the nucleotides in an oligonucleotide can be ribonucleotides, 2′-O-methyl ribonucleotides, or a mixture of ribonucleotides and 2′-O-methyl ribonucleotides; about 10% to about 50% of the nucleotides can be ribonucleotides, 2′-O-methyl ribonucleotides, or a mixture of ribonucleotides and 2′-O-methyl ribonucleotides; about 50% or more of the nucleotides can be ribonucleotides, 2′-O-methyl ribonucleotides, or a mixture of ribonucleotides and 2′-O-methyl ribonucleotides; or all of the nucleotides are ribonucleotides, 2′-O-methyl ribonucleotides, or a mixture of ribonucleotides and 2′-β-methyl ribonucleotides. Such oligonucleotides and nucleic acids can be referred to as chimeric oligonucleotides and chimeric nucleic acids.

### M. Solid Supports

Solid supports are solid-state substrates or supports with which molecules (such as trigger molecules) and riboswitches (or other components used in, or produced by, the disclosed methods) can be associated. Riboswitches and other molecules can be associated with solid supports directly or indirectly. For example, analytes (e.g., trigger molecules, test compounds) can be bound to the surface of a solid support or associated with capture agents (e.g., compounds or molecules that bind an analyte) immobilized on solid supports. As another example, riboswitches can be bound to the surface of a solid support or associated with probes immobilized on solid supports. An array is a solid support to which multiple riboswitches, probes or other molecules have been associated in an array, grid, or other organized pattern.

Solid-state substrates for use in solid supports can include any solid material with which components can be associated, directly or indirectly. This includes materials such as acrylamide, agarose, cellulose, nitrocellulose, glass, gold, polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, polylactic acid, polyorthoesters, functionalized silane, polypropylfumerate, collagen, glycosaminoglycans, and polyamino acids. Solid-state substrates can have any useful form including thin film, membrane, bottles, dishes, fibers, woven fibers, shaped polymers, particles, beads, microparticles, or a combination. Solid-state substrates and solid supports can be porous or non-porous. A chip is a rectangular or square small piece of material. Preferred forms for solid-state substrates are thin films, beads, or chips. A useful form for a solid-state substrate is a microtiter dish. In some embodiments, a multiwell glass slide can be employed.

An array can include a plurality of riboswitches, trigger molecules, other molecules, compounds or probes immobilized at identified or predefined locations on the solid support. Each predefined location on the solid support generally has one type of component (that is, all the components at that location are the same). Alternatively, multiple types of components can be immobilized in the same predefined location on a solid support. Each location will have multiple copies of the given components. The spatial separation of different components on the solid support allows separate detection and identification.

Although useful, it is not required that the solid support be a single unit or structure. A set of riboswitches, trigger molecules, other molecules, compounds and/or probes can be distributed over any number of solid supports. For example, at one extreme, each component can be immobilized in a separate reaction tube or container, or on separate beads or microparticles.

Methods for immobilization of oligonucleotides to solid-state substrates are well established. Oligonucleotides, including address probes and detection probes, can be coupled to substrates using established coupling methods. For example, suitable attachment methods are described by Pease et al., Proc. Natl. Acad. Sci. USA 91(11):5022-5026 (1994), and Khrapko et al., Mol Biol (Mosk) (USSR) 25:718-730 (1991). A method for immobilization of 3′-amine oligonucleotides on casein-coated slides is described by Stimpson et al., Proc. Natl. Acad. Sci. USA 92:6379-6383 (1995). A useful method of attaching oligonucleotides to solid-state substrates is described by Guo et al., Nucleic Acids Res. 22:5456-5465 (1994).

Each of the components (for example, riboswitches, trigger molecules, or other molecules) immobilized on the solid support can be located in a different predefined region of the solid support. The different locations can be different reaction chambers. Each of the different predefined regions can be physically separated from each other of the different regions. The distance between the different predefined regions of the solid support can be either fixed or variable. For example, in an array, each of the components can be arranged at fixed distances from each other, while components associated with beads will not be in a fixed spatial relationship. In particular, the use of multiple solid support units (for example, multiple beads) will result in variable distances.

Components can be associated or immobilized on a solid support at any density. Components can be immobilized to the solid support at a density exceeding 400 different components per cubic centimeter. Arrays of components can have any number of components. For example, an array can have at least 1,000 different components immobilized on the solid support, at least 10,000 different components immobilized on the solid support, at least 100,000 different components immobilized on the solid support, or at least 1,000,000 different components immobilized on the solid support.

### N. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits for detecting compounds, the kit comprising one or more biosensor riboswitches. The kits also can contain reagents and labels for detecting activation of the riboswitches.

### O. Mixtures

Disclosed are mixtures formed by performing or preparing to perform the disclosed method. For example, disclosed are mixtures comprising riboswitches and trigger molecules.

Whenever the method involves mixing or bringing into contact compositions or components or reagents, performing the method creates a number of different mixtures. For example, if the method includes 3 mixing steps, after each one of these steps a unique mixture is formed if the steps are performed separately. In addition, a mixture is formed at the completion of all of the steps regardless of how the steps were performed. The present disclosure contemplates these mixtures, obtained by the performance of the disclosed methods as well as mixtures containing any disclosed reagent, composition, or component, for example, disclosed herein.

### P. Systems

Disclosed are systems useful for performing, or aiding in the performance of, the disclosed method. Systems generally comprise combinations of articles of manufacture such as structures, machines, devices, and the like, and compositions, compounds, materials, and the like. Such combinations that are disclosed or that are apparent from the disclosure are contemplated. For example, disclosed and contemplated are systems comprising biosensor riboswitches, a solid support and a signal-reading device.

### Q. Data Structures and Computer Control

Disclosed are data structures used in, generated by, or generated from, the disclosed method. Data structures generally are any form of data, information, and/or objects collected, organized, stored, and/or embodied in a composition or medium. Riboswitch structures and activation measurements stored in electronic form, such as in RAM or on a storage disk, is a type of data structure.

The disclosed method, or any part thereof or preparation therefor, can be controlled, managed, or otherwise assisted by computer control. Such computer control can be accomplished by a computer controlled process or method, can use and/or generate data structures, and can use a computer program. Such computer control, computer controlled processes, data structures, and computer programs are contemplated and should be understood to be disclosed herein.

### Methods

Disclosed are methods of identifying compounds that activate, deactivate or block a riboswitch. For example, compounds that activate a riboswitch can be identified by bringing into contact a test compound and a riboswitch and assessing activation of the riboswitch. If the riboswitch is activated, the test compound is identified as a compound that activates the riboswitch. Activation of a riboswitch can be assessed in any suitable manner. For example, the riboswitch can be linked to a reporter RNA and expression, expression level, or change in expression level of the reporter RNA can be measured in the presence and absence of the test compound. As another example, the riboswitch can include a conformation dependent label, the signal from which changes depending on the activation state of the riboswitch. Such a riboswitch preferably uses an aptamer domain from or derived from a naturally occurring riboswitch. As can be seen, assessment of activation of a riboswitch can be performed with the use of a control assay or measurement or without the use of a control assay or measurement. Methods for identifying compounds that deactivate a riboswitch can be performed in analogous ways.

Identification of compounds that block a riboswitch can be accomplished in any suitable manner. For example, an assay can be performed for assessing activation or deactivation of a riboswitch in the presence of a compound known to activate or deactivate the riboswitch and in the presence of a test compound. If activation or deactivation is not observed as would be observed in the absence of the test compound, then the test compound is identified as a compound that blocks activation or deactivation of the riboswitch.

Multiple different approaches can be used to detect binding RNAs, including, for example, allosteric ribozyme assays using gel-based and chip-based detection methods, and in-line probing assays. High throughput testing can also be accomplished by using, for example, fluorescent detection methods. For example, the natural catalytic activity of a glucosamine-6-phosphate sensing riboswitch that controls gene expression by activating RNA-cleaving ribozyme can be used. This ribozyme can be reconfigured to cleave separate substrate molecules with multiple turnover kinetics. Therefore, a fluorescent group held in proximity to a quenching group can be uncoupled (and therefore become more fluorescent) if a compound triggers ribozyme function. Second, molecular beacon technology can be employed. This creates a system that suppresses fluorescence if a compound prevents the beacon from docking to the riboswitch RNA. Either approach can be applied to any of the riboswitch classes by using RNA engineering strategies described herein.

High-throughput screening can also be used to reveal entirely new chemical scaffolds that also bind to riboswitch RNAs either with standard or non-standard modes of molecular recognition. Multiple different approaches can be used to detect metabolite binding RNAs, including allosteric ribozyme assays using gel-based and chip-based detection methods, and in-line probing assays. Also disclosed are compounds made by identifying a compound that activates, deactivates or blocks a riboswitch and manufacturing the identified compound. This can be accomplished by, for example, combining compound identification methods as disclosed elsewhere herein with methods for manufacturing the identified compounds. For example, compounds can be made by bringing into contact a test compound and a riboswitch, assessing activation of the riboswitch, and, if the riboswitch is activated by the test compound, manufacturing the test compound that activates the riboswitch as the compound.

Also disclosed are compounds made by checking activation, deactivation or blocking of a riboswitch by a compound and manufacturing the checked compound. This can be accomplished by, for example, combining compound activation, deactivation or blocking assessment methods as disclosed elsewhere herein with methods for manufacturing the checked compounds. For example, compounds can be made by bringing into contact a test compound and a riboswitch, assessing activation of the riboswitch, and, if the riboswitch is activated by the test compound, manufacturing the test compound that activates the riboswitch as the compound. Checking compounds for their ability to activate, deactivate or block a riboswitch refers to both identification of compounds previously unknown to activate, deactivate or block a riboswitch and to assessing the ability of a compound to activate, deactivate or block a riboswitch where the compound was already known to activate, deactivate or block the riboswitch.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

It should be understood that particular contacts and interactions (such as hydrogen bond donation or acceptance) described herein for compounds interacting with riboswitches are preferred but are not essential for interaction of a compound with a riboswitch. For example, compounds can interact with riboswitches with less affinity and/or specificity than compounds having the disclosed contacts and interactions. Further, different or additional functional groups on the compounds can introduce new, different and/or compensating contacts with the riboswitches. For example, for cyclic diGMP riboswitches, large or small functional groups can be used. Such functional groups can have, and can be designed to have, contacts and interactions with other part of the riboswitch. Such contacts and interactions can compensate for contacts and interactions of the trigger molecules and core structure. Useful functional groups can be attached, for example, to the alpha-carbon of cyclic diGMP. Modifications to the side chain, carboxy group, primary amino group, or a combination, of cyclic diGMP can be used or avoided.

Also disclosed are methods of killing or inhibiting the growth of bacteria. The method can comprise contacting the bacteria with a compound identified by any of the methods disclosed herein. The method can comprise selecting a compound identified by any of the methods disclosed herein and contacting the bacteria with the selected compound. Also disclosed are methods of inhibiting gene expression. The method can comprise bringing into contact a compound and a cell, where the compound is identified by any of the disclosed methods. Also disclosed are methods of inhibiting gene expression. The method can comprise bringing into contact a compound and a cell, where the compound is identified by any of the disclosed methods. The method can comprise selecting a compound identified by any of the methods disclosed herein and bringing into contact the compound and a cell. Also disclosed are methods of promoting gene expression. The method can comprise bringing into contact a compound and a cell, where the compound is identified by any of the disclosed methods. The method can comprise selecting a compound identified by any of the methods disclosed herein and bringing into contact the compound and a cell.

Also disclosed are methods comprising: (a) testing a compound identified by any of the disclosed methods for inhibition of gene expression of a gene encoding an RNA comprising a cyclic diGMP riboswitch, where the inhibition is via the riboswitch; and (b) inhibiting gene expression by bringing into contact a cell and a compound that inhibited gene expression in step (a). The cell can comprise a gene encoding an RNA comprising a target riboswitch, where the target riboswitch is a cyclic diGMP riboswitch, where the compound inhibits expression of the gene by binding to the target riboswitch. The riboswitch can comprise a cyclic di-GMP-II motif.

Also disclosed are methods for activating, deactivating or blocking a riboswitch. Such methods can involve, for example, bringing into contact a riboswitch and a compound or trigger molecule that can activate, deactivate or block the riboswitch. Riboswitches function to control gene expression through the binding or removal of a trigger molecule. Compounds can be used to activate, deactivate or block a riboswitch. The trigger molecule for a riboswitch (as well as other activating compounds) can be used to activate a riboswitch. Compounds other than the trigger molecule generally can be used to deactivate or block a riboswitch. Riboswitches can also be deactivated by, for example, removing trigger molecules from the presence of the riboswitch. Thus, the disclosed method of deactivating a riboswitch can involve, for example, removing a trigger molecule (or other activating compound) from the presence or contact with the riboswitch. A riboswitch can be blocked by, for example, binding of an analog of the trigger molecule that does not activate the riboswitch. The method can comprise selecting a compound or trigger molecule that can activate, deactivate or block a riboswitch and bringing into contact the riboswitch and the selected compound or trigger molecule. The method can comprise selecting a compound identified by any of the disclosed methods that can activate, deactivate or block a riboswitch and bringing into contact the riboswitch and the selected compound.

Also disclosed are methods for altering expression of an RNA molecule, or of a gene encoding an RNA molecule, where the RNA molecule includes a riboswitch, by bringing a compound into contact with the RNA molecule. Riboswitches function to control gene expression through the binding or removal of a trigger molecule. Thus, subjecting an RNA molecule of interest that includes a riboswitch to conditions that activate, deactivate or block the riboswitch can be used to alter expression of the RNA. Expression can be altered as a result of, for example, termination of transcription or blocking of ribosome binding to the RNA. Binding of a trigger molecule can, depending on the nature of the riboswitch, reduce or prevent expression of the RNA molecule or promote or increase expression of the RNA molecule. The method can comprise selecting a compound that can activate, deactivate or block a riboswitch and bringing into contact an RNA molecule comprising the riboswitch and the selected compound. The method can comprise selecting a compound identified by any of the disclosed methods that can activate, deactivate or block a riboswitch and bringing into contact an RNA molecule comprising the riboswitch and the selected compound.

Also disclosed are methods for regulating expression of a naturally occurring gene or RNA that contains a riboswitch by activating, deactivating or blocking the riboswitch. If the gene is essential for survival of a cell or organism that harbors it, activating, deactivating or blocking the riboswitch can result in death, stasis or debilitation of the cell or organism. For example, activating a naturally occurring riboswitch in a naturally occurring gene that is essential to survival of a microorganism can result in death of the microorganism (if activation of the riboswitch turns off or represses expression). This is one basis for the use of the disclosed compounds and methods for antimicrobial and antibiotic effects. The compounds that have these antimicrobial effects are considered to be bacteriostatic or bacteriocidal. The method can comprise selecting a compound that can activate, deactivate or block a riboswitch and bringing into contact a gene or RNA that contains the riboswitch and the selected compound. The method can comprise selecting a compound identified by any of the disclosed methods that can activate, deactivate or block a riboswitch and bringing into contact a gene or RNA that contains the riboswitch and the selected compound.

Also disclosed are methods for selecting and identifying compounds that can activate, deactivate or block a riboswitch. Activation of a riboswitch refers to the change in state of the riboswitch upon binding of a trigger molecule. A riboswitch can be activated by compounds other than the trigger molecule and in ways other than binding of a trigger molecule. The term trigger molecule is used herein to refer to molecules and compounds that can activate a riboswitch. This includes the natural or normal trigger molecule for the riboswitch and other compounds that can activate the riboswitch. Natural or normal trigger molecules are the trigger molecule for a given riboswitch in nature or, in the case of some non-natural riboswitches, the trigger molecule for which the riboswitch was designed or with which the riboswitch was selected (as in, for example, in vitro selection or in vitro evolution techniques). Non-natural trigger molecules can be referred to as non-natural trigger molecules.

Also disclosed are methods of killing or inhibiting bacteria or microorganisms, comprising contacting the bacteria or microorganisms with a compound disclosed herein or identified by the methods disclosed herein. The method can comprise selecting a compound identified by any of the methods disclosed herein and bringing into contact bacteria or microorganisms and the selected compound. The method can comprise selecting a compound identified by any of the methods disclosed herein and bringing into contact bacteria or microorganisms and the selected compound. The method can comprise selecting a compound that can activate, deactivate or block a riboswitch and bringing into contact bacteria or microorganisms and the selected compound. The method can comprise selecting a compound identified by any of the disclosed methods that can activate, deactivate or block a riboswitch and bringing into contact bacteria or microorganisms and the selected compound. The method can comprise selecting a compound that can activate, deactivate or block a riboswitch and bringing into contact bacteria or microorganisms that contain the riboswitch and the selected compound. The method can comprise selecting a compound identified by any of the disclosed methods that can activate, deactivate or block a riboswitch and bringing into contact bacteria or microorganisms that contain the riboswitch and the selected compound.

Also disclosed are methods of identifying compounds that activate, deactivate or block a riboswitch. For examples, compounds that activate a riboswitch can be identified by bringing into contact a test compound and a riboswitch and assessing activation of the riboswitch. If the riboswitch is activated, the test compound is identified as a compound that activates the riboswitch. Activation of a riboswitch can be assessed in any suitable manner. For example, the riboswitch can be linked to a reporter RNA and expression, expression level, or change in expression level of the reporter RNA can be measured in the presence and absence of the test compound. As another example, the riboswitch can include a conformation dependent label, the signal from which changes depending on the activation state of the riboswitch. Such a riboswitch preferably uses an aptamer domain from or derived from a naturally occurring riboswitch. As can be seen, assessment of activation of a riboswitch can be performed with the use of a control assay or measurement or without the use of a control assay or measurement. Methods for identifying compounds that deactivate a riboswitch can be performed in analogous ways.

In addition to the methods disclosed elsewhere herein, identification of compounds that block a riboswitch can be accomplished in any suitable manner. For example, an assay can be performed for assessing activation or deactivation of a riboswitch in the presence of a compound known to activate or deactivate the riboswitch and in the presence of a test compound. If activation or deactivation is not observed as would be observed in the absence of the test compound, then the test compound is identified as a compound that blocks activation or deactivation of the riboswitch.

Also disclosed are methods of detecting compounds using biosensor riboswitches. The method can include bringing into contact a test sample and a biosensor riboswitch and assessing the activation of the biosensor riboswitch. Activation of the biosensor riboswitch indicates the presence of the trigger molecule for the biosensor riboswitch in the test sample. Biosensor riboswitches are engineered riboswitches that produce a detectable signal in the presence of their cognate trigger molecule. Useful biosensor riboswitches can be triggered at or above threshold levels of the trigger molecules. Biosensor riboswitches can be designed for use in vivo or in vitro. For example, biosensor riboswitches operably linked to a reporter RNA that encodes a protein that serves as or is involved in producing a signal can be used in vivo by engineering a cell or organism to harbor a nucleic acid construct encoding the riboswitch/reporter RNA. An example of a biosensor riboswitch for use in vitro is a cyclic diGMP riboswitch that includes a conformation dependent label, the signal from which changes depending on the activation state of the riboswitch. Such a biosensor riboswitch preferably uses an aptamer domain from or derived from a naturally occurring cyclic diGMP riboswitch.

Also disclosed are compounds made by identifying a compound that activates, deactivates or blocks a riboswitch and manufacturing the identified compound. This can be accomplished by, for example, combining compound identification methods as disclosed elsewhere herein with methods for manufacturing the identified compounds. For example, compounds can be made by bringing into contact a test compound and a riboswitch, assessing activation of the riboswitch, and, if the riboswitch is activated by the test compound, manufacturing the test compound that activates the riboswitch as the compound.

Also disclosed are compounds made by checking activation, deactivation or blocking of a riboswitch by a compound and manufacturing the checked compound. This can be accomplished by, for example, combining compound activation, deactivation or blocking assessment methods as disclosed elsewhere herein with methods for manufacturing the checked compounds. For example, compounds can be made by bringing into contact a test compound and a riboswitch, assessing activation of the riboswitch, and, if the riboswitch is activated by the test compound, manufacturing the test compound that activates the riboswitch as the compound. Checking compounds for their ability to activate, deactivate or block a riboswitch refers to both identification of compounds previously unknown to activate, deactivate or block a riboswitch and to assessing the ability of a compound to activate, deactivate or block a riboswitch where the compound was already known to activate, deactivate or block the riboswitch.

Disclosed is a method of detecting a compound of interest, the method comprising bringing into contact a sample and a cyclic diGMP riboswitch, where the riboswitch is activated by the compound of interest, where the riboswitch produces a signal when activated by the compound of interest, where the riboswitch produces a signal when the sample contains the compound of interest. The riboswitch can change conformation when activated by the compound of interest, where the change in conformation produces a signal via a conformation dependent label. The riboswitch can change conformation when activated by the compound of interest, where the change in conformation causes a change in expression of an RNA linked to the riboswitch, where the change in expression produces a signal. The signal can be produced by a reporter protein expressed from the RNA linked to the riboswitch.

Disclosed is a method comprising (a) testing a compound for inhibition of gene expression of a gene encoding an RNA comprising a riboswitch, where the inhibition is via the riboswitch, and (b) inhibiting gene expression by bringing into contact a cell and a compound that inhibited gene expression in step (a), where the cell comprises a gene encoding an RNA comprising a riboswitch, where the compound inhibits expression of the gene by binding to the riboswitch.

### A. Identification of Antimicrobial Compounds

Riboswitches are a class of structured RNAs that have evolved for the purpose of binding small organic molecules. The natural binding pocket of riboswitches can be targeted with metabolite analogs or by compounds that mimic the shape-space of the natural metabolite. The small molecule ligands of riboswitches provide useful sites for derivitization to produce drug candidates. Distribution of some riboswitches is shown in Table 1 of U.S. Application Publication No. 2005-0053951. Once a class of riboswitch has been identified and its potential as a drug target assessed, such as the cyclic diGMP riboswitch, candidate molecules can be identified.

The emergence of drug-resistant stains of bacteria highlights the need for the identification of new classes of antibiotics. Anti-riboswitch drugs represent a mode of anti-bacterial action that is of considerable interest for the following reasons. Riboswitches control the expression of genes that are critical for fundamental metabolic processes. Therefore manipulation of these gene control elements with drugs yields new antibiotics. These antimicrobial agents can be considered to be bacteriostatic, or bacteriocidal. Riboswitches also carry RNA structures that have evolved to selectively bind metabolites, and therefore these RNA receptors make good drug targets as do protein enzymes and receptors. Furthermore, it has been shown that two antimicrobial compounds (discussed above) kill bacteria by deactivating the antibiotics resistance to emerge through mutation of the RNA target.

### B. Methods of Using Antimicrobial Compounds

Disclosed herein are in vivo and in vitro anti-bacterial methods. By “anti-bacterial” is meant inhibiting or preventing bacterial growth, killing bacteria, or reducing the number of bacteria. Thus, disclosed is a method of inhibiting or preventing bacterial growth comprising contacting a bacterium with an effective amount of one or more compounds disclosed herein. Additional structures for the disclosed compounds are provided herein.

Disclosed herein is also a method of inhibiting growth of a cell, such as a bacterial cell or a microbial cell, that is in a subject, the method comprising administering an effective amount of a compound as disclosed herein to the subject. This can result in the compound being brought into contact with the cell. The subject can have, for example, a bacterial infection, and the bacterial cells can be inhibited by the compound. The bacteria can be any bacteria, such as cyanobacteria or bacteria from the genus Bacillus or Staphylococcus, for example. Bacterial growth can also be inhibited in any context in which bacteria are found. For example, bacterial growth in fluids, biofilms, and on surfaces can be inhibited. The compounds disclosed herein can be administered or used in combination with any other compound or composition. For example, the disclosed compounds can be administered or used in combination with another antimicrobial compound.

The bacteria can be any bacteria, such as bacteria from the genus Bacillus, Acinetobacter, Actinobacillus, Alkaliphilus, Clostridium, Dehalococcides, Deinococcus, Desulfitobacterium, Enterococcus, Erwinia, Escherichia, Exiguobacterium, Fusobacterium, Geobacillus, Haemophilus, Halothermothrix, Klebsiella, Idiomarina, Lactobacillus, Lactococcus, Leuconostoc, Listeria, Moorella, Mycobacterium, Oceanobacillus, Oenococcus, Pasteurella, Pediococcus, Pelotomaculum, Pseudomonas, Shewanella, Shigella, Solibacter, Staphylococcus, Streptococcus, Thermoanaerobacter, Thermosinus, Thermotoga, and Vibrio, for example. The bacteria can be, for example, Actinobacillus pleuropneumoniae, Alkaliphilus metalliredigens, Bacillus anthracis, Bacillus cereus, Bacillus clausii, Bacillus halodurans, Bacillus licheniformis, Bacillus subtilis, Bacillus thuringiensis, Clostridium acetobutylicum, Clostridiumbeijerinckii, Clostridiumbutyricum, Clostridium dificile, Clostridium kluyveri, Clostridium novyi, Clostridium perfringens, Clostridium tetani, Clostridium thermocellum, Deinococcus geothermalis, Deinococcus radiodurans, Desulfitobacterium hafniense, Enterococcus faecalis, Erwinia carotovora, Escherichia coli, Exiguobacterium sp., Fusobacterium nucleatum, Geobacillus kaustophilus, Haemophilus ducreyi, Haemophilus influenzae, Haemophilus somnus, Halothermothrix orenii, Idiomarina loihiensis, Lactobacillus acidophilus, Lactobacillus casei, Lactobacillus delbrueckii, Lactobacillus gasseri, Lactobacillus johnsonii, Lactobacillus plantarum, Lactococcus lactis, Leuconostoc mesenteroides, Listeria innocua, Listeria monocytogenes, Moorella thermoacetica, Oceanobacillus iheyensis, Oenococcus oeni, Pasteurella multocida, Pediococcus pentosaceus, Pelotomaculum thermopropionicum, Shewanella oneidensis, Shigella flexneri, Solibacter usitatus, Staphylococcus aureus, Staphylococcus epidermidis, Thermoanaerobacter ethanolicus, Thermoanaerobacter tengcongensis, Thermosinus carboxydivorans, Thermotoga maritima, Vibrio cholerae, Vibrio fischeri, Vibrio parahaemolyticus, or Vibrio vulnificus.

Bacterial growth can also be inhibited in any context in which bacteria are found. For example, bacterial growth in fluids, biofilms, and on surfaces can be inhibited. The compounds disclosed herein can be administered or used in combination with any other compound or composition. For example, the disclosed compounds can be administered or used in combination with another antimicrobial compound.

“Inhibiting bacterial growth” is defined as reducing the ability of a single bacterium to divide into daughter cells, or reducing the ability of a population of bacteria to form daughter cells. The ability of the bacteria to reproduce can be reduced by about 10%, about 20%, about 30%, about 40%, about 50%, about 60%, about 70%, about 80%, about 90%, or 100% or more.

Also provided is a method of killing a bacterium or population of bacteria comprising contacting the bacterium with one or more of the compounds disclosed and described herein.

“Killing a bacterium” is defined as causing the death of a single bacterium, or reducing the number of a plurality of bacteria, such as those in a colony. When the bacteria are referred to in the plural form, the “killing of bacteria” is defined as cell death of a given population of bacteria at the rate of 10% of the population, 20% of the population, 30% of the population, 40% of the population, 50% of the population, 60% of the population, 70% of the population, 80% of the population, 90% of the population, or less than or equal to 100% of the population.

The compounds and compositions disclosed herein have anti-bacterial activity in vitro or in vivo, and can be used in conjunction with other compounds or compositions, which can be bacteriocidal as well.

By the term “therapeutically effective amount” of a compound as provided herein is meant a nontoxic but sufficient amount of the compound to provide the desired reduction in one or more symptoms. As will be pointed out below, the exact amount of the compound required will vary from subject to subject, depending on the species, age, and general condition of the subject, the severity of the disease that is being treated, the particular compound used, its mode of administration, and the like. Thus, it is not possible to specify an exact “effective amount.” However, an appropriate effective amount may be determined by one of ordinary skill in the art using only routine experimentation.

The compositions and compounds disclosed herein can be administered in vivo in a pharmaceutically acceptable carrier. By “pharmaceutically acceptable” is meant a material that is not biologically or otherwise undesirable, i.e., the material may be administered to a subject without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions or compounds disclosed herein can be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition or compounds, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The compositions and compounds disclosed herein can be used therapeutically in combination with a pharmaceutically acceptable carrier. Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

Therapeutic compositions as disclosed herein may also be delivered by the use of monoclonal antibodies as individual carriers to which the compound molecules are coupled. The therapeutic compositions of the present disclosure may also be coupled with soluble polymers as targetable drug carriers. Such polymers can include, but are not limited to, polyvinyl-pyrrolidone, pyran copolymer, polyhydroxypropylmethacryl-amidephenol, polyhydroxyethylaspartamidephenol, or polyethyl-eneoxidepolylysine substituted with palmitoyl residues. Furthermore, the therapeutic compositions of the present disclosure may be coupled to a class of biodegradable polymers useful in achieving controlled release of a drug, for example, polylactic acid, polyepsilon caprolactone, polyhydroxy butyric acid, polyorthoesters, polyacetals, polydihydro-pyrans, polycyanoacrylates and cross-linked or amphipathic block copolymers of hydrogels.

Preferably at least about 3%, more preferably about 10%, more preferably about 20%, more preferably about 30%, more preferably about 50%, more preferably 75% and even more preferably about 100% of the bacterial infection is reduced due to the administration of the compound. A reduction in the infection is determined by such parameters as reduced white blood cell count, reduced fever, reduced inflammation, reduced number of bacteria, or reduction in other indicators of bacterial infection. To increase the percentage of bacterial infection reduction, the dosage can increase to the most effective level that remains non-toxic to the subject.

As used throughout, “subject” refers to an individual. Preferably, the subject is a mammal such as a non-human mammal or a primate, and, more preferably, a human. “Subjects” can include domesticated animals (such as cats, dogs, etc.), livestock (e.g., cattle, horses, pigs, sheep, goats, etc.), laboratory animals (e.g., mouse, rabbit, rat, guinea pig, etc.) and fish.

A “bacterial infection” is defined as the presence of bacteria in a subject or sample. Such bacteria can be an outgrowth of naturally occurring bacteria in or on the subject or sample, or can be due to the invasion of a foreign organism.

The compounds disclosed herein can be used in the same manner as antibiotics. Uses of antibiotics are well established in the art. One example of their use includes treatment of animals. When needed, the disclosed compounds can be administered to the animal via injection or through feed or water, usually with the professional guidance of a veterinarian or nutritionist. They are delivered to animals either individually or in groups, depending on the circumstances such as disease severity and animal species. Treatment and care of the entire herd or flock may be necessary if all animals are of similar immune status and all are exposed to the same disease-causing microorganism.

Another example of a use for the compounds includes reducing a microbial infection of an aquatic animal, comprising the steps of selecting an aquatic animal having a microbial infection, providing an antimicrobial solution comprising a compound as disclosed, chelating agents such as EDTA, TRIENE, adding a pH buffering agent to the solution and adjusting the pH thereof to a value of between about 7.0 and about 9.0, immersing the aquatic animal in the solution and leaving the aquatic animal therein for a period that is effective to reduce the microbial burden of the animal, removing the aquatic animal from the solution and returning the animal to water not containing the solution. The immersion of the aquatic animal in the solution containing the EDTA, a compound as disclosed, and TRIENE and pH buffering agent may be repeated until the microbial burden of the animal is eliminated. (U.S. Pat. No. 6,518,252).

Other uses of the compounds disclosed herein include, but are not limited to, dental treatments and purification of water (this can include municipal water, sewage treatment systems, potable and non-potable water supplies, and hatcheries, for example).

### EXAMPLES

### A. Example 1

### Class II Cyclic di-GMP Riboswitches and Natural Allosteric Ribozymes

1. Materials and Methods

i. Reagents, Oligonucleotides, Plasmids and Bacteria

c-di-GMP, pGpG and 3′,5′-cyclic GMP were obtained from the BioLog Life Science Institute. ApG, GpG, GpA, pGpA, and pApA were purchased from Oligos Etc. and 3′-guanosine monophosphate was purchased from MP Biomedicals. All other chemicals were purchased from Sigma-Aldrich. Synthetic oligonucleotides were obtained from either Sigma-Genosys or the W.M. Keck Foundation Biotechnology Resource Laboratory at Yale University.

pCR2.1 was purchased as a component of the TOPO TA cloning kit from Invitrogen. pDG1661 and B. subtilis strain 1A1 were obtained from the Bacillus Genetic Stock Center (BGSC; The Ohio State University). C. difficile 630 genomic DNA was obtained from ATCC (ATCC number: BAA-1382D-5). Competent TOP10 E. coli were obtained from Invitrogen. C. difficile 630 spores used to initiate cultures were obtained as a gift from Linc Sonenshein (Tufts University).

ii. Bioinformatics

Computer aided searches for new riboswitch candidates and the subsequent analysis of consensus sequence and structural models were conducted as reported previously (Weiberg et al., Nature 463:656 (2009); Weiberg et al., Genome Biol. 11:R31 (2010)).

iii. Preparation of RNAs

Wild-type DNA for the preparation of tandem riboswitch-ribozyme RNAs was amplified by PCR from the genomic DNA of Clostridium difficile 630. Primers were designed to include the promoter sequence for T7 RNA polymerase (T7 RNAP) in addition to two guanine residues at the start site of transcription to increase RNA yields. DNA was cloned into pCR2.1 using a TOPO TA cloning kit and TOP10 E. coli cells (Invitrogen) following the manufacturer's protocol. Mutants were obtained by using appropriately-mutated primers during PCR, and the resulting PCR products were cloned as described above. Nucleotide sequences of the resulting constructs were verified by DNA sequencing (W.M. Keck Foundation DNA sequencing facility). All primers used to generate wild-type and mutant tandem DNAs are listed in Table 4.

DNA templates for in vitro RNA transcription were obtained by PCR amplification of wild-type or mutant pCR2.1 plasmids prepared as described above. The full-length templates were transcribed in vitro using bacteriophage T7 RNA polymerase (T7 RNAP) in 80 mM N-(2-hydroxyethyl)piperazine-M-(2-ethanesulfonic acid) (HEPES) (pH 7.5 at 25° C.), 40 mM dithiothreitol (DTT), 6 mM MgCl₂, 2 mM spermidine, and 2 mM of each nucleoside 5′-triphosphate (NTP). Note that Mg²′ concentrations are reduced compared to typical transcription reactions to restrict ribozyme splicing during production. For preparation of internally ³²P-labeled RNAs, transcription reaction mixtures were supplemented with 0.5 μCi/μL [α-³²P]UTP. Shorter templates (smaller than 250 nucleotides and lacking ribozyme activity) were transcribed in vitro with T7 RNAP under the same conditions as described above, except that 24 mM MgCl₂ was used. RNA was purified using denaturing (8 M urea) 6% polyacrylamide gel electrophoresis (PAGE). Desired RNAs were isolated by excising and crushing the appropriate product band, and eluting the RNA with 10 mM Tris-HCl (pH 7.5 at 23° C.), 200 mM NaCl, and 1 mM EDTA (pH 8.0), followed by precipitation with ethanol.

5′ ³²P-labeled RNAs were prepared by first removing the 5′-terminal phosphates using alkaline phosphatase (Roche Diagnostics) following the manufacturer's protocol. RNAs were then radiolabeled using [γ-³²P]ATP and T4 polynucleotide kinase (New England Biolabs). 5′ ³²P-labeled RNAs were gel purified and precipitated as described above.

iv. In-Line Probing

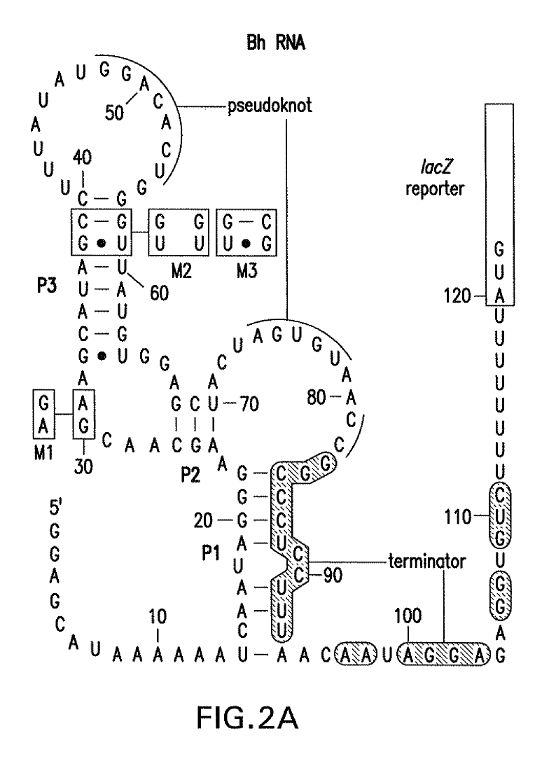

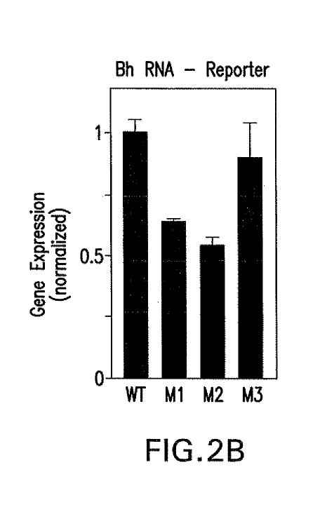

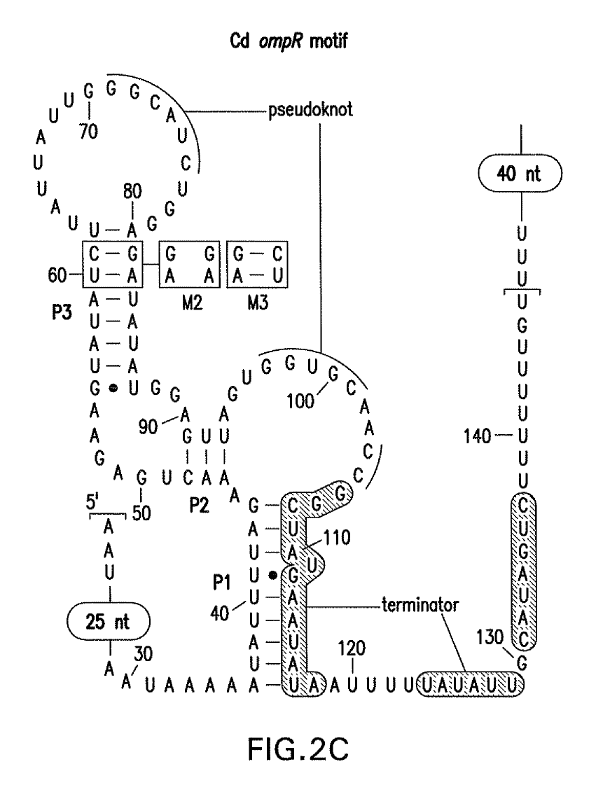

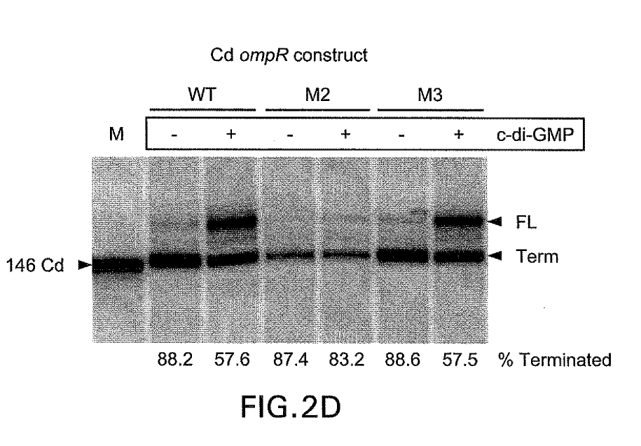

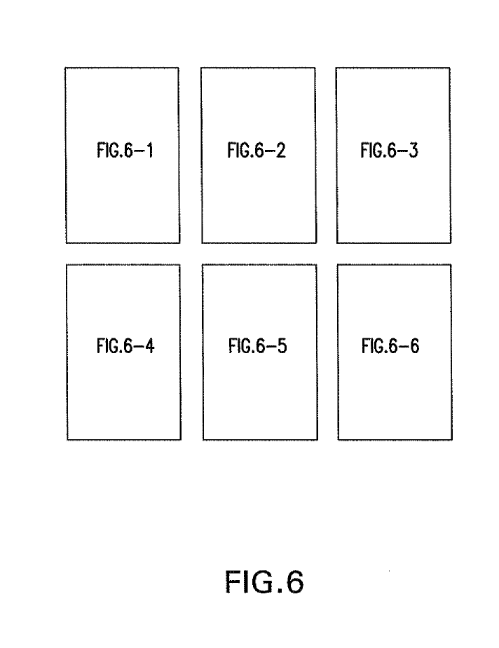

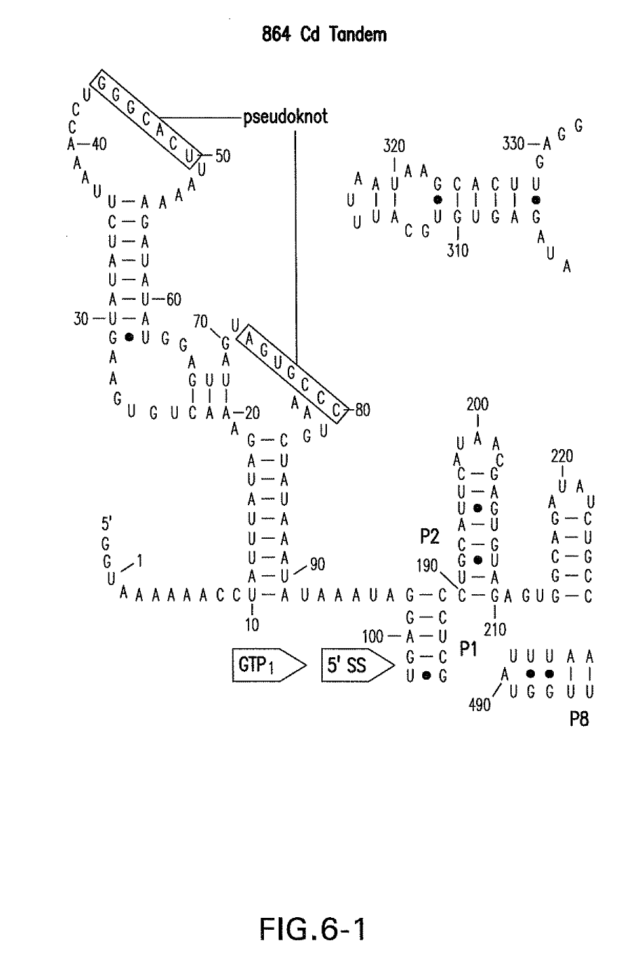

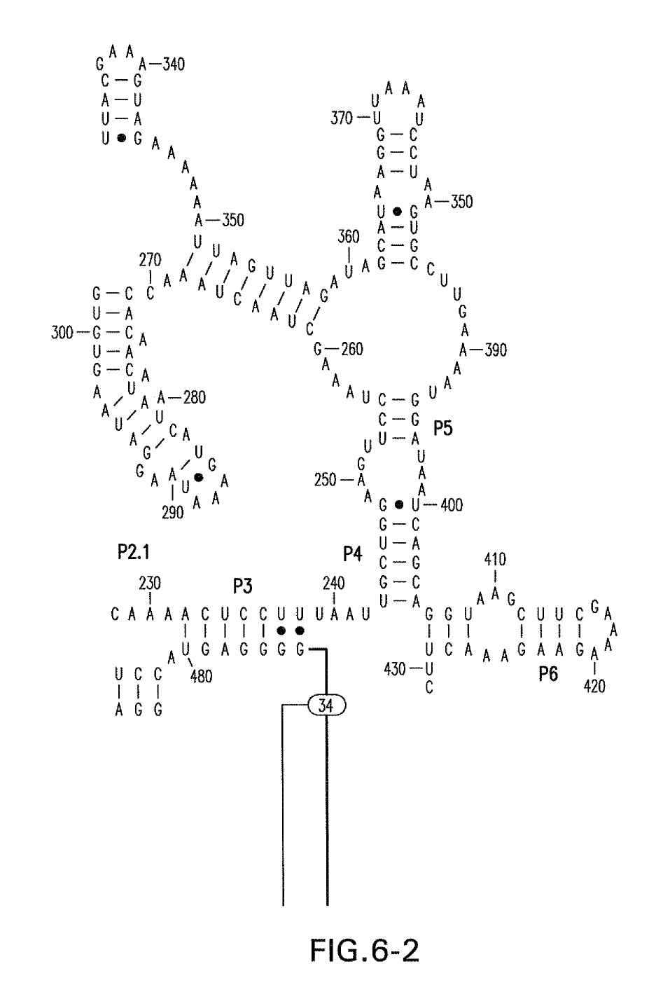

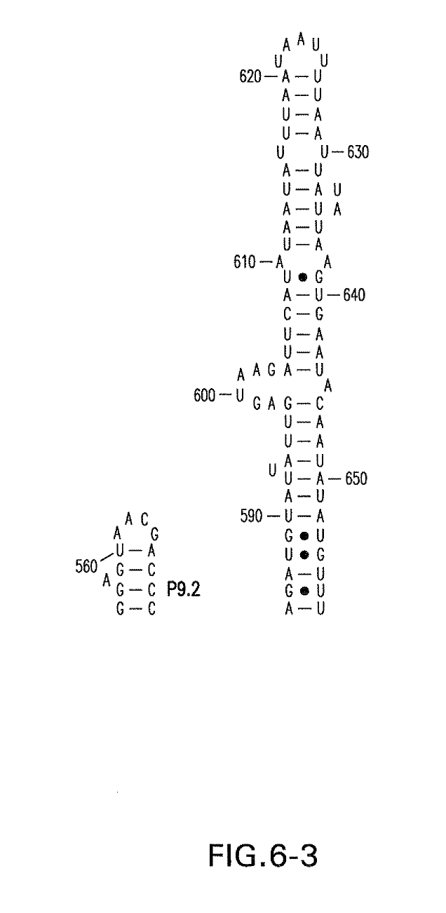

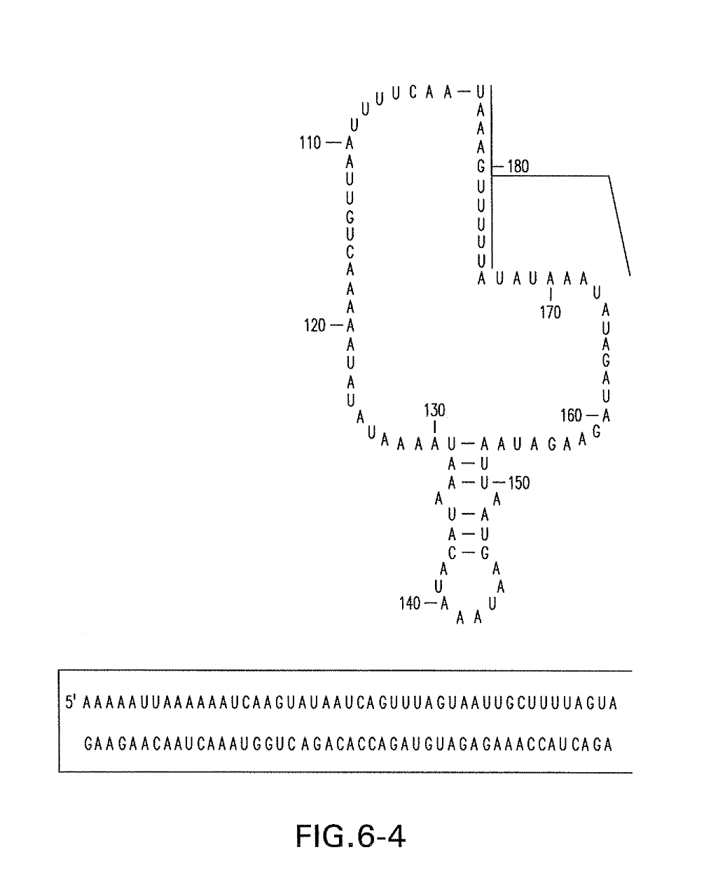

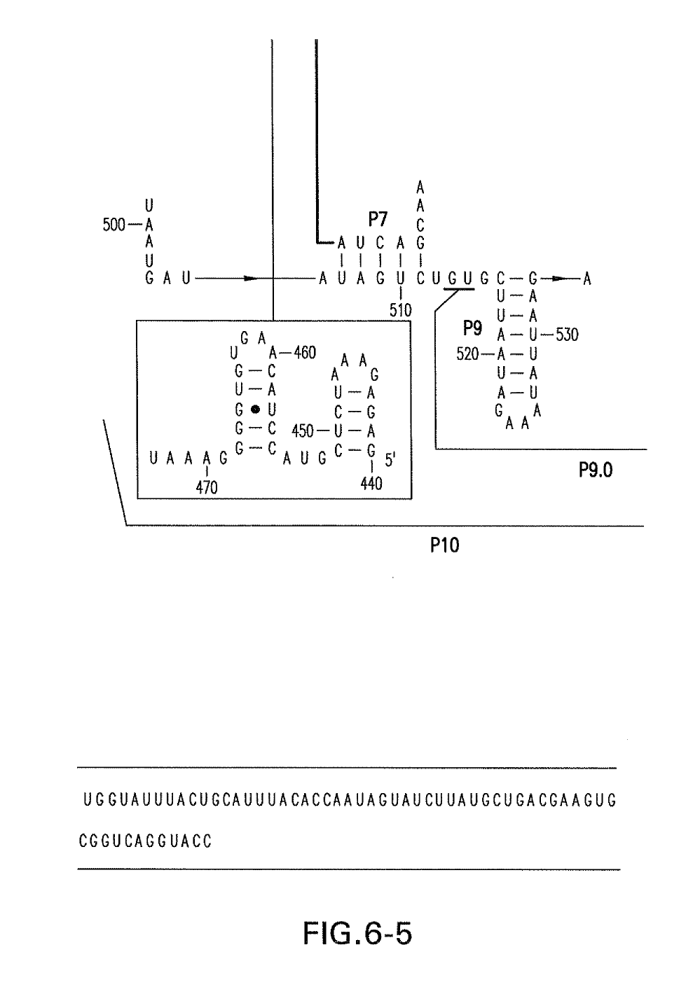

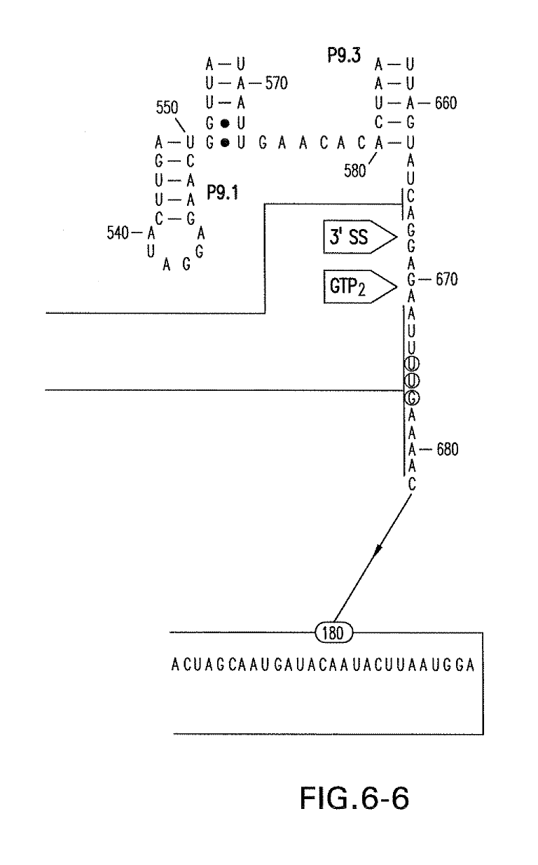

In-line probing exploits the instability of RNA phosphodiester linkages in unstructured portions of an RNA polymer to yield ligand-binding and structural information. In-line probing reactions were carried out largely as described previously (FIG. 2). Briefly, 5′ ³²P-labeled RNA (˜100 μM) was incubated at room temperature (˜23° C.) for approximately 40 hours in 100 mM KCl, 50 mM Tris-HCl (pH 8.3 at 23° C.), and 20 mM MgCl₂. Cleavage products were separated by denaturing 10% PAGE. Gels were dried and imaged using a PhosphorImager (Molecular Dynamics). Analyses of band intensities were carried out using SAFA v1.1 software (FIG. 6). This data was corrected for differences in radioactive sample loading per lane. Areas of band-intensity modulation were identified (designated as 1, 2, and 3 in FIG. 1C and data not shown) and the intensities were normalized and plotted against the logarithm of the concentration of ligand (M), as shown in FIG. 1D. Concentrations of c-di-GMP used for in-line probing of the 84 Cd RNA ranged from 1.2 μM to 260 fM with 1:3 dilutions. The apparent KD of 200 μM for 84 Cd RNA approaches the limit of sensitivity of the assay, and therefore the true affinity may be even better. In-line probing with various analogs used concentrations of linear pGpG, GpG, and ApG ranging from 100 μM to 2.3 μM with 1:3 dilutions. Concentrations of c-di-GMP used for in-line probing of the 132 Cd RNA ranged from 1.2 μM to 260 fM with 1:9 dilutions. Analysis of mutant 132 Cd RNAs used c-di-GMP concentrations from 1 μM to 100 μM with 1:10 dilutions. Dissociation constant (KD) values were determined by a best fit of the data to a standard 1:1 binding curve equation. The standard binding curve is represented by the equation F=[L]/([L]+K_{D}), where F and [L] represent the normalized fraction of RNA modulated and ligand concentration (M), respectively.

v. B. halodurans Reporter Constructs

DNAs corresponding to the 5′ UTR from the BH0361 locus of B. halodurans encompassing the c-di-GMP aptamer and the corresponding expression platform were created by PCR with two overlapping synthetic oligonucleotides that span the entire region. The native promoter was replaced with the promoter from the B. subtilis lysC gene to separate the effects of the promoter from the aptamer function. To do this, a synthetic oligonucleotide containing sequences corresponding to the lysC promoter and part of the c-di-GMP-II aptamer was used in a subsequent PCR amplification. EcoRI and BamHI restriction sites were included at the 5′ ends of the oligonucleotides to facilitate ligation using quick ligase (NEB) into pDG1661 after restriction digestion (NEB) and PCR purification (Qiagen) according to the manufacturers' protocols. Ligated plasmid was transformed into TOP10 cells (Invitrogen) and plated on Luria-Bertani (LB) agar plates containing 100 μg/mL carbenicillin. Plasmid DNA was collected from single-colony transformed cells grown in liquid LB medium supplemented with 100 μg/mL carbenicillin using a QIAprep Spin Miniprep kit (Qiagen) according to the manufacturer's protocol. The wild-type construct was verified by DNA sequencing (W.M. Keck Foundation DNA sequencing facility) and then used as a PCR template for the preparation of mutants as described above.

Wild-type and mutant Bha-pDG1661 plasmids were transformed into the genome of B. subtilis by double-crossover homologous recombination at the amyE locus. B. subtilis strain 1A1 was grown to an OD600 of between 0.4 and 0.8. Approximately 0.5 μg of each plasmid was added to 1 mL of the culture and grown with shaking for 40 minutes at 37° C. 2XYT medium was added and the cultures were grown as before for 45 minutes before plating 200 μl on TBAB plates containing 5 μg/mL chloramphenicol. Single colonies were tested for double-crossover events by verifying that they did not grow on 100 μg/mL spectinomycin. The spectinomycin-resistance gene in pDG1661 will only remain if a single crossover event occurs.

vi. β-galactosidase Assays

Strains of B. subtilis containing wild-type or mutant B. halodurans BH0361 locus aptamers fused to an E. coli lacZ gene were grown overnight in 2XYT medium containing 5 μg/mL chloramphenicol at 37° C. with shaking Cultures were diluted to an OD600 of approximately 0.2 and grown for approximately 3 hours before performing β-galactosidase assays using the standard protocol.

vii. Transcription Termination Assays

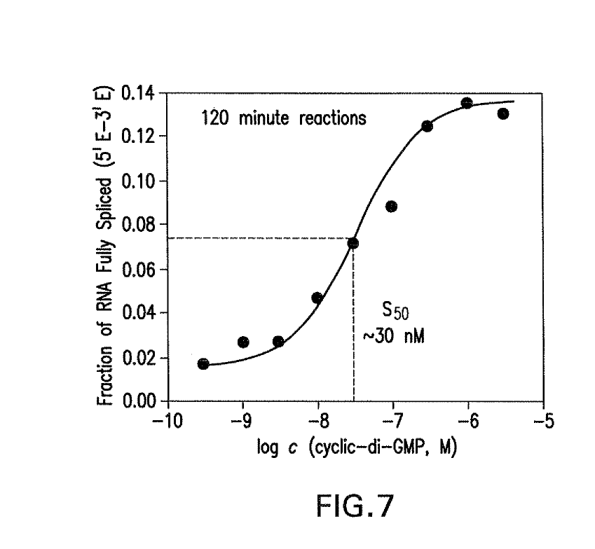

Single round transcription assays were conducted using a method (FIG. 7) adapted as described previously (data not shown) with the following modifications. The elongation mixture contained 50 μM UTP, and 150 μM each of ATP, GTP and CTP. Heparin was added at 0.1 mg/mL at initiation of elongation. For the reactions containing the second messenger, c-di-GMP was added to a final concentration of 50 μM.

A template DNA harboring the 5′ UTR of the ompR gene of C. difficile and its native promoter was prepared by PCR (see Table 4). The predicted intrinsic transcription termination site is 40 nucleotides shorter than the terminus of the template DNA. The ompR riboswitch is predicted to be an ‘ON’ switch based on its architecture. Therefore the full length transcript was expected to undergo decreased termination in the presence of c-di-GMP. Mutant templates M2 and M3 were constructed by PCR and confirmed by sequencing (see Table 4). A template DNA for production of a simulated intrinsic transcription termination product was generated by PCR and was used as a marker during PAGE analysis of transcription products.

viii. Splicing Assays of Wild-Type and Mutant RNAs

Initial ribozyme self-splicing reaction conditions were based on those previously reported for use with a group I ribozyme from Tetrahymena thermophile and consisted of incubations at 30° C. in the presence of 50 mM HEPES (pH 7.5 at 23° C.), 100 mM (NH₄)₂SO₄, 5 mM MgCl₂, and 0.1 mM GTP. At concentrations of MgCl₂ above 10 mM and temperatures at 37° C. or above, the difference between the ligated exons and alternate GTP attack site products became less pronounced. A range of ligand concentrations were tested for both GTP (5 μM to 1 mM) and cyclic di-GMP (200 μM to 100 μM) to determine the optimal amounts for splicing activity, based on probable biological relevance and degree of allosteric control.

Splicing assays for wild-type and mutant RNAs were carried out for 30 minutes (unless otherwise noted) under the conditions described above, with the addition of 50 μM GTP and/or 10 μM c-di-GMP when stated. Final RNA concentrations were lower than 1 μM for all reactions. Reaction mixtures containing unlabeled RNA were supplemented with 1 μCi/μL [α-³²P]GTP. Products were separated by denaturing 6% PAGE. Gels were dried and products were subsequently imaged using a PhosphorImager (Molecular Dynamics).

ix. Splicing Assays with [α-³²P]GTP to Find the 5′ Splice Site

Unlabeled RNA (˜5 μM) was subjected to the splicing reaction conditions described above. The reaction mixture was supplemented with 2.5 μCi/μL [α-³²P]GTP to radiolabel cleavage products derived from GTP attack. The reaction mixtures were incubated at 30° C. for 1 to 2 hours. Products were separated by denaturing 6% PAGE and RNA products were recovered from the gel as described above. The purified products carried [α-³²P]GTP at their 5′ terminus and therefore could be directly analyzed using partial digests with RNase T1 and alkali as described for the marker lanes for in-line probing assays (FIG. 2). Products of the partial digestion reactions were separated by denaturing 20% PAGE and the resulting gels were imaged using a PhosphorImager (Molecular Dynamics).

x. Splicing Assays to Determine Rate Constants and the S₅₀

Aliquots from splicing reactions conducted with either 5′ or internally ³²P-labeled 864 Cd Tandem RNAs were removed at various incubation times and the reactions were terminated by the addition of stop buffer (see below). Similarly, time course experiments were conducted with unlabeled 864 Cd Tandem RNAs supplemented with ˜50 nCi/μL [α-³²P]GTP. Reactions were initiated by combining a solution containing RNA only (˜100 pM 5′ ³²P-labeled RNA, ˜1 μM internally ³²P-labeled RNA, or ˜10 nM unlabeled RNA) with a solution containing the buffer and any ligands to yield a final concentration of contents as described above. The two mixtures were briefly pre-heated at 30° C. and the reaction was initiated by the transfer of buffer/ligand solution to the RNA solution. The zero time point was removed prior to initiation of the reaction and immediately placed into an equivalent volume of stop buffer (8 M urea, 20% w/v sucrose, 0.1% SDS, 0.05% bromophenol blue, 0.05% xylene cyanol FF, 0.09 M Tris base, 0.09 M borate, 1 mM EDTA [pH 8.0]). Likewise, subsequent aliquots were removed at each time point and quenched by placement in stop buffer. Reaction products were separated as described above and dried gels were imaged using a PhosphorImager (Molecular Dynamics).

For precursor RNAs that were initially ³²P-labeled, rate constants for product formation were established by determining the yield of specific products relative to the unprocessed or misprocessed RNAs. First, an average background for each lane was calculated and subtracted from intensities of individual bands or lanes. Second, product band intensities were compared to the total intensity of their corresponding lane, which corrected for any differences due to sample loading. Third, the band intensity at time zero was subtracted from the corresponding bands of all other time points. Yields of products from assays conducted with internally ³²P-labeled RNAs were also corrected for the number of uridine nucleotides in the product RNA versus the total number of uridine nucleotides in the precursor RNA (252 nucleotides). This correction allows for direct comparison of the number of molecules present in each product band. The total fraction of RNA processed at 120 minutes is ˜40%. However, the yields do not increase substantively at these longer incubation times, and therefore we speculate that approximately half of the precursor RNAs are unable to react (perhaps due to misfolding problems). This extent of RNA misfolding, particularly in the absense of possible protein factors, is also typical of other large functional RNAs. Thus, at 120 minutes, approximately 80% of the total reactable RNA has undergone processing. The fractions of RNA processed for the internally ³²P-labeled RNA was adjusted to reflect this value of 80% at 120 minutes.

For splicing assays conducted using unlabeled precursor RNAs, determination of rate constants were conducted differently. To account for differences in lane loading, a loading correction factor was calculated. This factor was BN/B1, where B1 and BN represent the background intensities for the first lane (time zero minutes) and the Nth lane, respectively. Lane intensities are likely proportional to the amount of [α-³²P]GTP loaded into each lane. The loading correction factor was multiplied by the intensity of the product band at time zero. This value was subtracted from the band intensities obtained for subsequent time points. The total fraction of the two products processed at 120 minutes was assumed to be 80% of the active RNA population, based on the estimations described above.

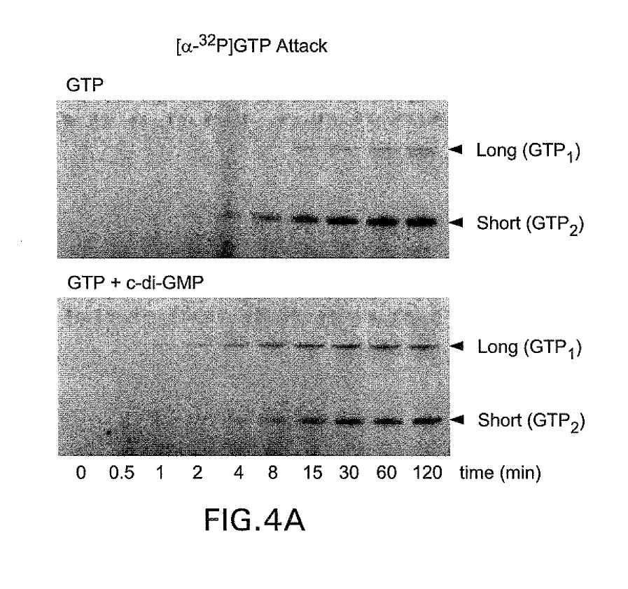

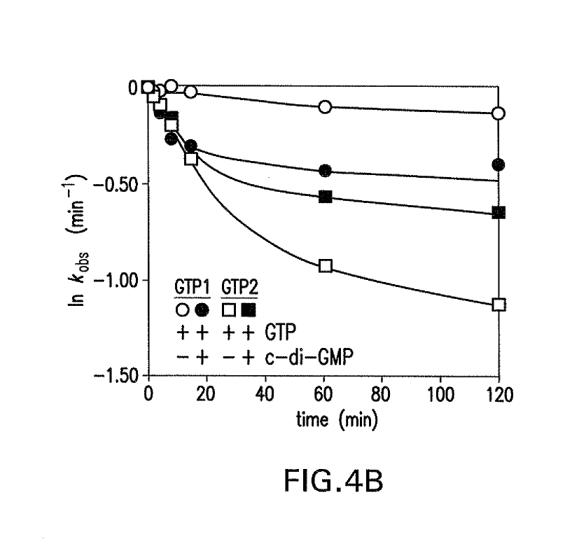

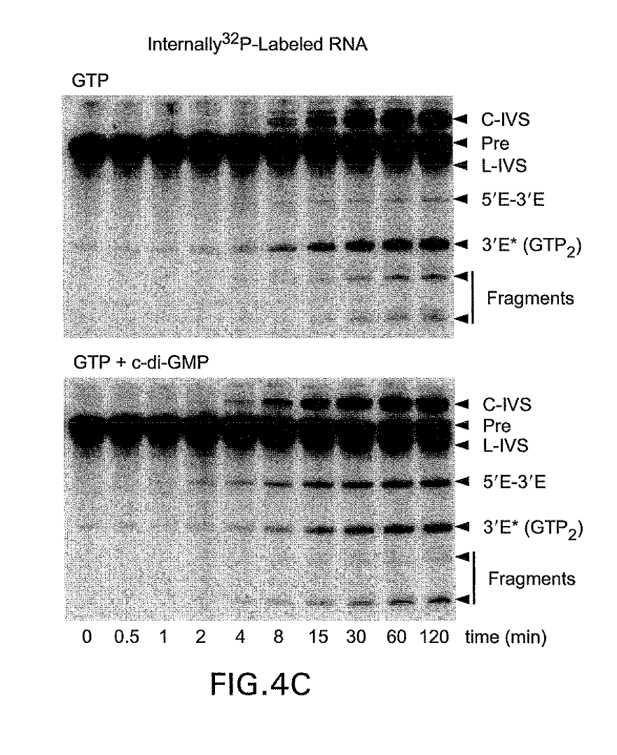

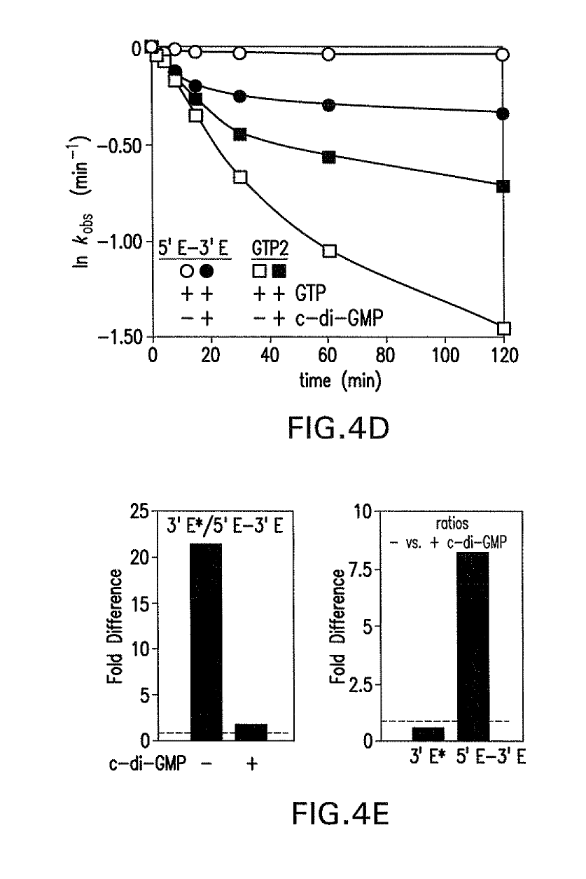

Rate constants (Table 5) were calculated by plotting the natural logarithm of the fraction of unreacted RNA versus time for the internally ³²P-labeled RNA and the unlabeled RNA assays. The rate constants were estimated by determining the negative slope of the resulting best fit line through the data points from the first 15 minutes of each assay. The resulting plots are presented in FIGS. 4B and 4D.

The S₅₀, defined as the concentration of c-di-GMP at which the 5′E-3′E is half-maximally processed, was determined by conducting assays using 5′ ³²P-labeled 864 Cd Tandem RNA. The fraction of the 5′E-3′E processed at 120 minutes was plotted versus the logarithm of the concentration of cyclic di-GMP, ranging from 300 μM to 3 μM. The S₅₀ is ˜30 nM.

xi. Growth of Clostridium difficile 630

Growth of C. difficile strain 630 cells were initiated from spore stock (gift of Dr. Linc Sonenshein, Tufts University), were maintained at 37° C. in a anaerobic chamber (Coy) on BHIS (Brain Heart Infusion Medium; obtained from BD Biosciences) agar plates supplemented with 0.1% cysteine, or grown in BHIS medium as described previously (Sorg and Dineen, Curr. Protoc. Microbiol. Ch. 9, Unit 9A.1 (2009)). For total RNA isolation, C. difficile cells were grown overnight in BHIS medium and were subcultured into fresh medium the next day. Aliquots of cells were drawn at different time points and were frozen at −80° C.

RNA was isolated using the Trizol LS reagent (Invitrogen) following the manufacturer's instructions.

xii. RT-PCR Analysis of Clostridium difficile 630 Splicing

In vivo splicing was examined by conducting reverse transcription and PCR of RNA samples isolated from C. difficile cells as described above. The total RNA isolated was treated with RNase-free DNase RQ1 (Promega) at 37° C. for two hours. After inactivation of DNase (65° C., 10 min), reverse transcription was carried out using ˜1.25 μg of RNA at 55° C. with Superscript III reverse transcriptase (Invitrogen) using primer 47 that anneals near the 5′ end within the ORF of CD3246 and downstream of the predicted group I intron 3′ splice site. To rule out the possibility of amplification from genomic DNA, identical control reactions were carried out without the addition of reverse transcriptase. Primer 48 anneals in the 5′ region of the c-di-GMP-II aptamer located in the 5′ UTR of the CD3246 gene, and was used with primer 47 for PCR.

The PCR product corresponding in size to the spliced exons was purified by agarose gel electrophoresis, cloned using a TA cloning kit (Invitrogen), and the splice product was confirmed by DNA sequencing.

xiii. RT-PCR Analysis of In Vitro Splice Products

Products of RNA processing were evaluated by conducting RT-PCR reactions with appropriate DNA primers and sequencing the resulting products. For example, RNAs corresponding to the putative spliced exons (5′E-3′E) were isolated by denaturing PAGE, and approximately 1 ng of RNA was used to make cDNA with SSII reverse transcriptase (Invitrogen) according to the manufacturer's protocol. The resulting cDNA was amplified by PCR using primers 3 and 37 (Table 4) and cloned into the TOPO pCR2.1 vector as described above. The sequence of the ligated exons was determined by DNA sequencing (W.M. Keck Foundation DNA sequencing facility).

Similarly, RT-PCR products were prepared and examined to establish sites of ribozyme circularization. Briefly, splicing reactions were carried out as described above and products were separated by denaturing 6% PAGE. This gel was run long enough to visualize three or four circularization products via autoradiography. An attempt was made to excise individual circularization product bands from the gel. RT-PCR experiments and cloning were carried out as described above on the excised product bands (GTP only lane). Approximately five single colonies were chosen from the colonies on each plate and their DNA was sequenced as noted above. Identical sequences were found on multiple plates, suggesting that separation of different circular RNA products was incomplete. Although there may be a bias toward certain products, our results verify the existence of the GTP2 attack site and the 3′SS.

TABLE 4
Sequences of DNA primers used tin this study.

| SEQ | | |
|---|---|---|
| ID | | |
| NO | DNA sequence (5′ to 3′) | Use |
| 1 | taatacgactcactataggtatttatagaaactgtgaag | In-line probing, 84 Cd |
| 2 | tatttatagcaggttgcactac | In-line probing, 84 Cd |
| 3 | tacgactcactataggtaaaaaacctatttatagaaactgtg | Constructing Cd-pCR2.1; in-line |
| | | probing, 132 Cd |
| 4 | ggtacctgaccgtctgatggtttc | Constructing Cd-pCR2.1 |
| 5 | taatacgactcactataggtaaaaaacctatttatagaaactgta | Two-step PCR, Cd-pCR2.1, M1 |
| | gagtatatcttaaacctgggcacttaaaag | |
| 6 | atatcttaaacctgggcacttaaaacttatatggagttagtagtgc | Two-step PCR, Cd-pCR2.1, M2 |
| | aacc | |
| 7 | ggttgcactactaactccatataagttttaagtgcccaggtttaag | Two-step PCR, Cd-pCR2.1, M2 |
| | atat | |
| 8 | aacctatttatagaaactgtgaagtataagttaaacctgggcactt | Two-step PCR, Cd-pCR2.1, M3 |
| | aaaacttatatg | |
| 9 | catataagttttaagtgcccaggtttaacttatacttcacagtttcta | Two-step PCR, Cd-pCR2.1, M3 |
| | taaataggtt | |
| 10 | taatacgactcactataggtaaaaaacctatggatagaaactgt | Two-step PCR, Cd-pCR2.1, M4 |
| | gaagtatatcttaaacc | |
| 11 | atatggagttagtagtgcaacctgctatccatataaataggagta | Two-step PCR, Cd-pCR2.1, M4 |
| | acttttaattgtc | |
| 12 | gacaattaaaagttactcctatttatatggatagcaggttgcacta | Two-step PCR, Cd-pCR2.1, M4 |
| | ctaactccatat | |
| 13 | tatatggagttagtagtgcaacctgctataaatatccataggagta | Two-step PCR, Cd-pCR2.1, M5 |
| | acttttaattgtc | |
| 14 | gacaattaaaagttactcctatggatatttatagcaggttgcacta | Two-step PCR, Cd-pCR2.1, M5 |
| | ctaactccatata | |
| 15 | gagttagtagtgcaacctgctatccatatccataggagtaactttt | Two-step PCR, Cd-pCR2.1, M6 |
| | aattgtc | |
| 16 | gacaattaaaagttactcctatggatatggatagcaggttgcact | Two-step PCR, Cd-pCR2.1, M6 |
| | actaactc | |
| 17 | gatagatataaatatatttttgaaatggtccctgcattcataacgag | Two-step PCR, Cd-pCR2.1, M7 |
| | tg | |
| 18 | cactcgttatgaatgcagggaccatttcaaaaatatatttatatcta | Two-step PCR, Cd-pCR2.1, M7 |
| | tc | |
| 19 | gtagtgcaacctgctataaatataaataggactaacttttaattgtc | Two-step PCR, Cd-pCR2.1, M8 |
| | aaaaatatataaaataa | |
| 20 | ttattttatatatttttgacaattaaaagttagtcctatttatatttatag | Two-step PCR, Cd-pCR2.1, M8 |
| | caggttgcactac | |
| 21 | ttaagtgaatacaatatatgtttttagtatcaggacaattttgaaaa | Two-step PCR, Cd-pCR2.1, M9 |
| | caaaaattaaaaaatcaagtataa | |
| 22 | ttatacttgattttttaatttttgttttcaaaattgtcctgatactaaaaa | Two-step PCR, Cd-pCR2.1, M9 |
| | catatattgtattcacttaa | |
| 23 | ggagtacctaatttatggttaggtaatgatatagtttgtgcttaata | Two-step PCR, Cd-pCR2.1, M12 |
| | gaaatat | |
| 24 | atatttctattaagcacaaactatatcattacctaaccataaattag | Two-step PCR, Cd-pCR2.1, M12 |
| | gtactcc | |
| 25 | ttttatatatttttgacaattaaaagttactcc | In-line probing, 132 Cd |
| 26 | ggagcataaaaaatcaatagggaagcaacgaagcatagccttt | Constructing Bha-pDG1661 |
| | atatggacacttgggttatgtggagctactagtgtaaccggccct | |
| 27 | agttttcataaaaaaaagacacctctcctattgttaaaggagggc | Constructing Bha-pDG1661 |
| | cggttacactagtagctccacataacccaagtgtccatataaa | |
| 28 | tgagaattctacgacaaattgcaaaaataatgttgtcctttta | Constructing Bha-pDG1661, add lysC |
| | aataagatctgataaaatgtgaactaaggaggataaaaaat | |
| | caatagggaagc | |
| 29 | gatgaattctacgacaaattgcaaaaataatgttgtccttttaaata | lysC forward primer with EcoRI site |
| | agatctgataaaatgtgaac | |
| 30 | ctggatccagttttcataaaaaaaagacacc | Constructing Bha-pDG1661, reverse |
| | | primer with BamHI |
| 31 | gggaagcaacagagcatagcc | Two-step PCR, Bha-pDG1661, M1 |
| 32 | ggctatgctctgttgcttccc | Two-step PCR, Bha-pDG1661, M1 |
| 33 | agggaagcaacgaagcatatgctttatatggacacttggg | Two-step PCR, Bha-pDG1661, M2 |
| 34 | cccaagtgtccatataaagcatatgcttcgttgcttccct | Two-step PCR, Bha-pDG1661, M2 |
| 35 | tatgctttatatggacacttggcgtatgtggagctactagtgtaac | Two-step PCR, Bha-pDG1661, M3 |
| 36 | gttacactagtagctccacatacgccaagtgtccatataaagcat | Two-step PCR, Bha-pDG1661, M3 |
| | a | |
| 37 | ggtacctgaccgtctgatggtttctctacatctggtgtctgaccat | RT-PCR, reverse for ligated exons |
| | ttgattgttcttctcc | |
| 38 | gaccctaattgaacacactaaagatgtattattgagtaagattc | RT-PCR, circular product |
| 39 | ctaattttttctactttcgtaacctcaagtgcttattaaatgc | RT-PCR, circular product |
| 40 | atcttatatctaagaatatggaaata | For amplifying the ompR 5′ UTR from |
| | | C. difficile, forward primer |
| 41 | tactcttattttcaaattttgcaacatttttgttaatttt | For amplifying the ompR 5′ UTR, |
| | | reverse primer that anneals 40 nts from |
| | | the predicted terminator site |
| 42 | acaaaaaaagactatgcaatataaaaattatattcat | Reverse primer for the ompR 5′ UTR |
| | | that anneals near the predicted |
| | | terminator |
| 43 | ttttagaaactgagaagtataagttattattgggcatctggag | Forward Primer that introduces the M1 |
| | | mutation in the ompR 5′ UTR by PCR |
| | | mutagenesis |
| 44 | ctccagatgcccaataataacttatacttctcagtttctaaaa | Reverse complement of Primer 43 for |
| | | introducing the M1 mutation in the |
| | | ompR 5′ UTR |
| 45 | ttttagaaactgagaagtataagttattattgggcatctggacttat | Forward primer that introduces the M2 |
| | atggagttagtggtgca | mutation in the ompR 5′ UTR by PCR |
| | | mutagenesis |
| 46 | tgcaccactaactccatataagtccagatgcccaataataacttat | Reverse complement of Primer 45 for |
| | acttctcagtttctaaaa | introducing the M2 mutation in the |
| | | ompR 5′ UTR |
| 47 | ccaatagtatcttatgctgacgaagtg | Primer for generating the cDNA for |
| | | RT-PCR analysis of splicing of |
| | | CD3246 region |
| 48 | gtgaagtatatcttaaacctgggcactt | Forward primer for RT-PCR analysis |
| | | of splicing of CD3246 region |

The T7 RNAP promoter is shown in italics, lysine promoter is shown in bold, and the restriction sites are underlined.

TABLE 5
Rate constants calculated for unlabeled
and internally ³²P-labeled RNA.
Unlabeled RNA

| GTP | c-di-GMP | Product | Rate constant (min⁻¹) |
|---|---|---|---|
| + | − | GTP1 | 1.8 × 10⁻³ |
| + | − | GTP2 | 2.7 × 10⁻² |
| + | + | GTP1 | 2.3 × 10⁻² |
| + | + | GTP2 | 2.3 × 10⁻² |

Internally ³²P-labeled RNA

| GTP | C-di-GMP | Product | Rate constant (min⁻¹) |
|---|---|---|---|
| + | − | 5′E-3′E | 1.1 × 10⁻³ |
| + | − | 3′E* | 2.4 × 10⁻² |
| + | + | 5′E-3′E | 1.3 × 10⁻² |
| + | + | 3′E* | 1.8 × 10⁻² |

TABLE 6
Genomic locations and gene associations for ten group I ribozymes in
Clostridium difficile. Representative 10 is the tandem riboswitch
ribozyme examined in this study.
Complement = ribozyme or gene is encoded on complementary strand.

Representative 1
Group I Intron Coordinates: 727944 to 728413 (Complement)
Adjacent to Transposase tlpB; Coordinates: 728498 to 728608
(Complement)
Group I intron interrupts clcA gene; Locus Tag CD0606; Coordinates:
727281 to 727943 and 728726 to 729637
Representative 2
Group I Intron Coordinates: 990470 to 990939
Adjacent to Transposase tlpB; Coordinates: 991118 to 992236
Group I intron interrupts the ptbA gene; Locus tag CD0816; Coordinates:
988673 to 990469 and 992354 to 992434
Representative 3
Group I Intron Coordinates: 1521545 to 1522014
Adjacent to Transposase tlpB; Coordinates: 1522193 to 1523311
Group I interrupts an ORF for putative RNA binding protein; Locus tag
CD1311; Coordinates: 1520876 to 1521544 and 1523429 to 1523734
Representative 4
Group I Intron Coordinates: 1736753 to 1737222
Adjacent to Transposase tlpB; Coordinates: 1737399 to 1738517
Group I interrupts the mviN gene; Locus tag CD1499; Coordinates:
1736355 to 1736753 and 1738636 to 1739781
Representative 5
Group I Intron Coordinates: 1818880 to 1819348
Adjacent to Transposase tlpA; Coordinates: 1819510 to 1819602
Group I intron interrupts an ORF for lysophospholipase; Locus tag
CD1570; Coordinates: 1818353 to 1818877 and 1820846 to 1821253
Representative 6
Group I Intron Coordinates: 1930135 to 1930604
Adjacent to Transposase tlpA; Coordinates: 1930766 to 1930873
Group I intron does not interrupt a gene.
Representative 7
Group I Intron Coordinates: 1992919 to 1993388
Adjacent to Transposase tlpB; Coordinates: 1993567 to 1994685
Group I intron interrupts an ORF involved in hydantoinase production;
Locus tag CD1718; Coordinates: 1992553 to 1992918 and 1994803 to
1995981
Representative 8
Group I Intron Coordinates: 2102880 to 2103349
Adjacent to Transposase tlpB; Coordinates: 2103561 to 2104646
Group I intron interrupts the adeC gene; Locus tag CD1820;
Coordinates: 2102703 to 2102879 and 2104764 to 2106284
Representative 9
Group I Intron Coordinates: 2954861 to 2955331 (Complement)
The Group I intron is inserted within the Transposase tlpB gene;
Locus tag CD2553
Representative 10
Group I Intron Coordinates: 3800624 to 3801050 (Complement)
Immediately adjacent to c-di-GMP aptamer; CDG-II aptamer boundary
coordinates: 3801145 to 3801070 (Complement)
Downstream (~100 nt) ORF is CD3246; Coordinates: 3800518 to
379822 (Complement)

2. Results

i. A New Aptamer Class for a Bacterial Second Messenger

A computational pipeline (Yao et al. PLoS Comput. Biol. 3, e126 (2007)) was employed to identify many candidate riboswitches and other RNA motifs in bacteria and archaea (Weinberg et al. Genome Biol.). Among several classes of riboswitch candidates identified is one (FIG. 1A) whose representatives are sometimes associated with bacterial genes involved in c-di-GMP production, degradation, and signaling (Tables 1, 2, and 3). 28 organisms from the class Clostridia or from the genus Deinococcus carry a total of 45 examples of this newfound c-di-GMP riboswitch. The conserved sequences and structural features are distinct from a previously validated riboswitch class (N. Sudarsan, et al Science 321, 411 (2008); N. Kulshina, et al. Nat. Struct. Mol. Biol. 16, 1212 (2009); K. D. et al. Nat. Struct. Mol. Biol. 16, 1218 (2009)), hereafter termed c-di-GMP-I riboswitches (FIG. 5), that was recently demonstrated (N. Sudarsan, et al Science 321, 411 (2008)) to control gene expression in response to changing concentrations of this second messenger. Table 1 shows a summary of organisms containing c-di-GMP-II riboswitches. Each riboswitch is assigned an abbreviation to identify it in Table 2 and Table 7. For example, the abbreviation “Dsp-1-1” denotes the first (and sole) predicted c-di-GMP riboswitch in Dehalococcoides sp. CBDB1. The full taxonomy of each organism is also listed.

TABLE 1
Summary of organisms containing predicted
c-di-GMP-II riboswitches.

| Abbrevia- | |
|---|---|
| tion | Taxonomy of species |
| Dsp-1-1 | Bacteria Chloroflexi Dehalococcoidetes Dehalococcoides |
| | sp. CBDB1 |
| Dge-1-1 | Bacteria Deinococcus-Thermus Deinococci Deinococcales |
| | Deinococcaceae Deinococcus geothermalis DSM 11300 |
| Dra-1-1 | Bacteria Deinococcus-Thermus Deinococci Deinococcales |
| to Dra-1-2 | Deinococcaceae Deinococcus radiodurans R1 |
| Bha-1-1 | Bacteria Firmicutes Bacillales Bacillaceae Bacillus halodurans |
| | C-125 |
| Bsp-1-1 | Bacteria Firmicutes Bacillales Bacillaceae Bacillus sp. NRRL |
| | B-14911 |
| Tca-1-1 | Bacteria Firmicutes Clostridia Clostridiales |
| | Acidaminococcaceae Thermosinus carboxydivorans Nor1 |
| Ame-1-1 | Bacteria Firmicutes Clostridia Clostridiales Clostridiaceae |
| | Alkaliphilus metalliredigens QYMF |
| Cac-1-1 | Bacteria Firmicutes Clostridia Clostridiales Clostridiaceae |
| to Cac-1-2 | Clostridium acetobutylicum ATCC 824 |
| Cbe-1-1 | Bacteria Firmicutes Clostridia Clostridiales Clostridiaceae |
| to Cbe-1-6 | Clostridium beijerinckii NCIMB 8052 |
| Cbu-1-1 | Bacteria Firmicutes Clostridia Clostridiales Clostridiaceae |
| | Clostridium butyricum 5521 |
| Cdi-1-1 | Bacteria Firmicutes Clostridia Clostridiales Clostridiaceae |
| to Cdi-1-4 | Clostridium difficile 630 |
| Cdi-2-1 | Bacteria Firmicutes Clostridia Clostridiales Clostridiaceae |
| to Cdi-2-3 | Clostridium difficile QCD-32g58 |
| Cdi-3-1 | Bacteria Firmicutes Clostridia Clostridiales Clostridiaceae |
| to Cdi-3-3 | Clostridium difficile QCD-66c26 |
| Ckl-1-1 | Bacteria Firmicutes Clostridia Clostridiales Clostridiaceae |
| | Clostridium kluyveri DSM 555 |
| Cno-1-1 | Bacteria Firmicutes Clostridia Clostridiales Clostridiaceae |
| | Clostridium novyi NT |
| Cpe-1-1 | Bacteria Firmicutes Clostridia Clostridiales Clostridiaceae |
| | Clostridium perfringens ATCC 13124 |
| Cpe-2-1 | Bacteria Firmicutes Clostridia Clostridiales Clostridiaceae |
| | Clostridium perfringens B str. ATCC 3626 |
| Cpe-3-1 | Bacteria Firmicutes Clostridia Clostridiales Clostridiaceae |
| | Clostridium perfringens C str. JGS1495 |
| Cpe-4-1 | Bacteria Firmicutes Clostridia Clostridiales Clostridiaceae |
| | Clostridium perfringens CPE str. F4969 |
| Cpe-5-1 | Bacteria Firmicutes Clostridia Clostridiales Clostridiaceae |
| | Clostridium perfringens E str. JGS1987 |
| Cpe-6-1 | Bacteria Firmicutes Clostridia Clostridiales Clostridiaceae |
| | Clostridium perfringens |

Table 2 shows the genes located downstream of predicted c-di-GMP-II riboswitches. All predicted c-di-GMP-II riboswitches (denoted “c-di-GMP-II→>”) are listed, with their nucleotide coordinates and downstream genes. The listed genes are derived from the annotation of the given sequence. Some sequences lack annotated genes, because no annotation was performed (e.g., the sequence containing Hor-1-1) or because only fragments were sequenced (e.g., the sequence containing env-3), and no gene is listed for such sequences. The directions of each riboswitch and downstream gene are indicated with an arrow (→). Conserved protein domains predicted to be encoded downstream of more than one c-di-GMP-II riboswitch are shown, and a brief description of each domain is provided in Table 3. Conserved domains were analyzed using the Conserved Domain Database. The accession number of the nucleotide sequence containing each c-di-GMP-II riboswitch is given, and the nucleotide numbers corresponding to the 5′ and 3′ ends of each predicted riboswitch aptamer is provided. The riboswitches env-4 and env-8 are adjacent to one another, and are depicted on the same line.

Conserved domains that were referenced in Table 2 are briefly described in Table 3, using text from the Conserved Domain Database.

TABLE 2
Genes located downstream of predicted c-di-GMP-II riboswitches

| Abbrev. | Sequence accession | | 5′ at | 3′ at | Genes |
|---|---|---|---|---|---|
| env-1 | soil_Contig17117 | + | 115 | 211 | c-di-GMP-II→Putative multicopper |
| | | | | | oxidases→ |
| env-2 | soil_XZS94218_g1 | − | 559 | 457 | c-di-GMP-II→hypo→ |
| env-3 | soil_XZS76186_x1 | − | 413 | 329 | c-di-GMP-II→ |
| env-4 | JCVI_SCAF_109662713 | + | 343 | 428 | c-di-GMP-II→c-di-GMP-II→ ←ORF |
| | 2715 | | | | |
| env-8 | JCVI_SCAF_109662713 | + | 175 | 258 | (shown above) |
| | 2715 | | | | |
| Ame-1-1 | NC_009633.1 | − | 3560727 | 3560645 | c-di-GMP-II→ PulE |
| | | | | | (COG2804)→ PilT |
| | | | | | (COG2805)→ PulF |
| | | | | | (COG1459)→ ComGC (COG4537)→ |
| env-5 | JCVI_SCAF_110166856 | + | 1009 | 1091 | c-di-GMP-II→ |
| | 9280 | | | | |
| Tet-1-1 | NZ_AAKQ01000038.1 | + | 10227 | 10310 | c-di-GMP-II→ PulE |
| | | | | | (COG2804)→ PilT |
| | | | | | (COG2805)→ PulF |
| | | | | | (COG1459)→ general secretion pathway |
| | | | | | protein H→ leader peptidase (prepilin |
| | | | | | peptidase)/N-methyltransferase→ |
| Tet-2-1 | NZ_AATV01000050.1 | − | 4408 | 4325 | c-di-GMP-II→ PulE |
| | | | | | (COG2804)→ PilT |
| | | | | | (COG2805)→ PulF (COG1459)→ |
| Pth-1-1 | NC_009454.1 | + | 2908378 | 2908465 | c-di-GMP-II→ COG4886 |
| | | | | | (COG4886)SLH (pfam00395)→ |
| Dra-1-1 | NC_001263.1 | + | 692045 | 692129 | c-di-GMP-II→ hypo→ |
| Dge-1-1 | NC_008025.1 | + | 298588 | 298670 | c-di-GMP-II→ COG4719 (COG4719)→ |
| | | | | | Transposase_11 (pfam01609)NOG38674 |
| | | | | | (NOG38674)→ |
| Dra-1-2 | NC_001263.1 | − | 1919839 | 1919755 | c-di-GMP-II→ hypo→ hypo→ hypo→ |
| env-6 | soil_XZS84852_b1 | + | 24 | 124 | c-di-GMP-II→ |
| Bsp-1-1 | NZ_AAOX01000007.1 | + | 31274 | 31356 | c-di-GMP-II→ OmpR (COG0745)PleD |
| | | | | | (COG3706)→hypo→ COG1215 |
| | | | | | (COG1215)→ hypo→ REC (cd00156)→ |
| | | | | | OmpR (COG0745)VicK (COG5002)→ |
| Dsp-1-1 | NC_007356.1 | + | 1260708 | 1260792 | c-di-GMP-II→ hypo→ |
| Hor-1-1 | NZ_AAOZ01000061.1 | + | 5221 | 5304 | c-di-GMP-II→ |
| Bha-1-1 | NC_002570.2 | + | 387918 | 388001 | c-di-GMP-II→ COG4932 (COG4932)→ |
| | | | | | SrtA (COG3764)→ |
| Ckl-1-1 | NC_009706.1 | + | 1160200 | 1160286 | c-di-GMP-II→ DiS_P_DiS |
| | | | | | (pfam06750)→ |
| Cbu-1-1 | NZ_ABDT01000023.1 | − | 17918 | 17835 | c-di-GMP-II→ |
| env-7 | BAAW01001404.1 | − | 567 | 484 | c-di-GMP-II→ hypo→ |
| Cbe-1-1 | NC_009617.1 | − | 4369760 | 4369677 | c-di-GMP-II→ Tar (COG0840)→ |
| Cbe-1-2 | NC_009617.1 | + | 2207052 | 2207135 | c-di-GMP-II→ hypo→ |
| Cbe-1-3 | NC_009617.1 | − | 3552610 | 3552527 | c-di-GMP-II→ MA (smart00283)→ |
| Cbe-1-4 | NC_009617.1 | + | 2550212 | 2550295 | c-di-GMP-II→ COG3434 (COG3434)→ |
| Cdi-1-1 | NC_009089.1 | − | 3801149 | 3801064 | c-di-GMP-II→ group I intron → putative |
| | | | | | surface protein (pfam06346) → |
| Cdi-2-1 | NZ_AAML04000014.1 | − | 1152543 | 1152460 | c-di-GMP-II→ OmpR (COG0745)→ |
| | | | | | hypo→ |
| Cdi-3-1 | NZ_ABFD01000063.1 | + | 191103 | 191186 | c-di-GMP-II→ |
| Cdi-1-2 | NC_009089.1 | − | 3826693 | 3826610 | c-di-GMP-II→ OmpR (COG0745)→ |
| | | | | | BaeS (COG0642)→ OmpR (COG0745)→ |
| Cdi-2-2 | NZ_AAML04000014.1 | − | 638715 | 638630 | c-di-GMP-II→ Cna_B (pfam05738)→ |
| | | | | | hypo→ |
| Cdi-3-2 | NZ_ABFD01000057.1 | + | 13031 | 13116 | c-di-GMP-II→ |
| Cdi-1-3 | NC_009089.1 | − | 3306767 | 3306682 | c-di-GMP-II→ Cna_B (pfam05738)→ |
| Cpe-7-1 | NC_008262.1 | + | 175590 | 175673 | c-di-GMP-II→ Cna_B (pfam05738)→ |
| | | | | | Cna_B (pfam05738)→ |
| Cpe-1-1 | NC_008261.1 | + | 171340 | 171423 | c-di-GMP-II→ Cna_B (pfam05738)→ |
| | | | | | Cna_B (pfam05738)→ |
| Cpe-3-1 | NZ_ABDU01000018.1 | + | 14999 | 15082 | c-di-GMP-II→ |
| Cpe-5-1 | NZ_ABDW01000009.1 | − | 70792 | 70709 | c-di-GMP-II→ |
| Cpe-6-1 | NZ_ABDY01000009.1 | + | 15518 | 15601 | c-di-GMP-II→ |
| Cpe-2-1 | NZ_ABDV01000011.1 | + | 27277 | 27360 | c-di-GMP-II→ |
| Cpe-8-1 | NC_003366.1 | + | 197260 | 197343 | c-di-GMP-II→ Cna_B (pfam05738)→ |
| | | | | | Cna_B (pfam05738→ |
| Cpe-4-1 | NZ_ABDX01000006.1 | − | 72290 | 72207 | c-di-GMP-II→ |
| Cdi-1-4 | NC_009089.1 | − | 4105882 | 4105797 | c-di-GMP-II→ pilin→ |
| Cdi-2-3 | NZ_AAML04000015.1 | − | 157646 | 157561 | c-di-GMP-II→ hypo→ |
| Cdi-3-3 | NZ_ABFD01000065.1 | − | 57289 | 57204 | c-di-GMP-II→ |
| Cac-1-1 | NC_003030.1 | − | 3752962 | 3752879 | c-di-GMP-II→ Probable S-layer protein→ |
| Cno-1-1 | NC_008593.1 | − | 1875256 | 1875172 | c-di-GMP-II→ Cna_B (pfam05738)→ |
| | | | | | Sortase (cd00004)→ |
| Cte-1-1 | NC_004557.1 | + | 474745 | 474827 | c-di-GMP-II→ Collagen_bind |
| | | | | | (pfam05737)Cna_B (pfam05738)→ |
| Cbe-1-5 | NC_009617.1 | − | 4868650 | 4868568 | c-di-GMP-II→ DiS_P_DiS |
| | | | | | (pfam06750)→ |
| Cbe-1-6 | NC_009617.1 | + | 1272636 | 1272718 | c-di-GMP-II→ hypo→ |
| Cac-1-2 | NC_003030.1 | + | 648479 | 648561 | c-di-GMP-II→ Cellulase_N_term |
| | | | | | (cd02850)Glyco_hydro_9 |
| | | | | | (pfam00759)CBM_4_9 |
| | | | | | (pfam02018)NOG07884 (NOG07884)→ |
| env-9 | soil_XZS39253_g1 | − | 485 | 401 | c-di-GMP-II→ |
| Tca-1-1 | NZ_AAWL01000006.1 | − | 67971 | 67888 | c-di-GMP-II→ PulE (COG2804)→ PilT |
| | | | | | (COG2805)→ PulF |
| | | | | | (COG1459)→ PulG |
| | | | | | (COG2165)→ DiS_P_DiS |
| | | | | | (pfam06750)→ FimT |
| | | | | | (COG4970)→ hypo→ hypo→ hypo |
| | | | | | → hypo (cons)→ PilM |
| | | | | | (COG4972)→ PilN |
| | | | | | (pfam05137)→ PilO (COG3167)→ |

TABLE 3
Conserved protein domains encoded by genes downstream of c-di-GMP-II riboswitches.

| domain | description |
|---|---|
| cd00004 | Sortase domain; transpeptidase of Gram-positive bacteria, cleaves surface proteins at |
| | the LPXTG motif between Thr and Gly and catalyzes the formation of an amide bond |
| | between the carboxyl group of Thr and the amino group of cell-wall crossbridges. |
| cd00156 | Signal receiver domain; originally thought to be unique to bacteria (CheY, OmpR, |
| | NtrC, and PhoB), now recently identified in eukaroytes ETR1 Arabidopsis thaliana; |
| | this domain receives the signal from the sensor partner in a two-component systems; |
| | contains a phosphoacceptor site that is phosphorylated by histidine kinase homologs; |
| | usually found N-terminal to a DNA binding effector domain; forms homodimers |
| cd02850 | Cellulase N-terminus domain. |
| COG0642 | Signal transduction histidine kinase [Signal transduction mechanisms] |
| COG0745 | Response regulators consisting of a CheY-like receiver domain and a winged-helix |
| | DNA-binding domain [Signal transduction mechanisms/Transcription] |
| COG0840 | Methyl-accepting chemotaxis protein [Cell motility and secretion/Signal |
| | transduction mechanisms] |
| COG1215 | Glycosyltransferases, probably involved in cell wall biogenesis [Cell envelope |
| | biogenesis, outer membrane] |
| COG1459 | Type II secretory pathway, component PulF [Cell motility and secretion/ |
| | Intracellular trafficking and secretion] |
| COG2165 | Type II secretory pathway, pseudopilin PulG [Cell motility and secretion/ |
| | Intracellular trafficking and secretion] |
| COG2804 | Type II secretory pathway, ATPase PulE/Tfp pilus assembly pathway, ATPase PilB |
| | [Cell motility and secretion/Intracellular trafficking and secretion] |
| COG2805 | Tfp pilus assembly protein, pilus retraction ATPase PilT [Cell motility and secretion/ |
| | Intracellular trafficking and secretion] |
| COG3167 | Tfp pilus assembly protein PilO [Cell motility and secretion/Intracellular trafficking |
| | and secretion] |
| COG3434 | Predicted signal transduction protein containing EAL and modified HD-GYP |
| | domains [Signal transduction mechanisms] |
| COG3706 | Response regulator containing a CheY-like receiver domain and a GGDEF domain |
| | [Signal transduction mechanisms] |
| COG3764 | Sortase (surface protein transpeptidase) [Cell envelope biogenesis, outer membrane] |
| COG4537 | Competence protein ComGC [Intracellular trafficking and secretion] |
| COG4719 | Uncharacterized protein conserved in bacteria [Function unknown] |
| COG4886 | Leucine-rich repeat (LRR) protein [Function unknown] |
| COG4932 | Predicted outer membrane protein [Cell envelope biogenesis, outer membrane] |
| COG4970 | Tfp pilus assembly protein FimT [Cell motility and secretion/Intracellular |
| | trafficking and secretion] |
| COG4972 | Tfp pilus assembly protein, ATPase PilM [Cell motility and secretion/Intracellular |
| | trafficking and secretion] |
| COG5002 | Signal transduction histidine kinase [Signal transduction mechanisms] |

Table 7. Multiple sequence alignment of c-di-GMP-II riboswitch aptamers, with unaligned flanking sequence (SEQ ID NOs:76 to 121). Nucleotides in columns that form base pairs in the consensus structure are indicated with < - - - - - > at the top of the sequences. These basepaired regions constitute Watson-Crick or G-U pairs unless marked as indicated below. P1 and P2 stems are at alignment positions 98-112, 186-188, 203-213. P3 stems are at alignment positions 120-129 and 174-181. Pseudoknots are at alignment positions 138-145 and 192-200. The modular hairpin within L3 is at alignment position 153-170. The regions defined by P1, P2, P3 and L3 are marked in FIG. 5. Nucleotide pairs that are not Watson-Crick or G-U are in bold. Nucleotides in the basepaired regions that are not basepaired shade.

Nucleotides predicted to form the stem of a rho-independent transcription terminator are underlined with a solid line. These nucleotides include bulges within the stem. Predicted start codons within annotated sequences are underlined with a dotted line, and nucleotides that perfectly fit consensus −35 and −10 box sequences are double underlined. Nucleotides at positions 1-97 and 214-509 within the sequences are outside of the conserved motif that roughly corresponds to the c-di-GMP-II riboswitch aptamer structure. Riboswitches Cdi-3-1, Cdi-3-3, Cpe-3-1, Cpe-5-1, Cpe-6-1 and Tet-2-1 are identical in sequence to other riboswitches, and are not shown in the alignment below.

The last five rows in each alignment block depict certain structures. Columns that normally form base pairs are also indicated below each block of the multiple-sequence alignment by angle brackets, wherein matching <and > symbols mark base-paired columns. Below these symbols, the number 2 marks columns exhibiting covariation, 1 indicates compatible mutations, 0 indicates invariant nucleotides, and a question mark indicates that the nucleotides are non-canonical in more than 10% of the sequences.

The consensus sequence is also shown below each block of the multiple-sequence alignment. Nucleotides at positions 117, 118, 134, 138, 139, 145, 185, 186, 190, 192, 196, 200, 201, and 204 of the alignment are conserved in at least 97% of sequences. Nucleotides at positions 108, 111, 129, 137, 141, 174, 187, 188, 193, 198, 199, and 203 of the alignment are conserved in at least 90% of sequences. Nucleotides at positions 1, 107, 109, 110, 112, 113, 116, 121-125, 130, 133, 135, 140, 142-144, 146, 148-173, 178-180, 182 189, 191, 194, 195, 197, 202 205, 227, 138, 508-509 of the alignment are conserved in at least 75% of sequences. The symbol N stands for a nucleotide whose identity is not conserved. Nucleotides at positions 3-104, 106, 114, 115, 119, 120, 126-128, 131, 136, 147, 175-177, 181, 183, 205, 206, 209-225, 227-236, 238-426 of the alignments are present in 97% or more sequences. Nucleotides at positions 2, 105, 207, 208, 427-507 of the alignment are present in 90% or more sequences. The nucleotide at position 132 is present in 75% or more sequences. Nucleotides at positions 107-112, 116-118, 121-123, 129, 130, 134, 135, 137-141, 143-146, 178-180, 182, 184-204, 226, and 237 are present in 50% or more sequences, while those present in less than 50% of the sequences are located at positions 1, 113, 124, 125, 142, 148-173, and 508-509.

Alignment positions 1-85:

| | 1 10 20 30 40 50 60 70 80 |
|---|---|
| env-1 | .CACUUUGGUACAUACAUUCACAACCACUAAAAUAGAUUAAGAAAGUUAAAAAGACCUUAUUUGUUAUCAAUAACGGUCUGGACA |
| env-2 | .AUCUGCCCAGGAGCGCACGUAGGCCGGCCGGUUCUCGAUUAGCACGCCAUCACGGUCCAGGAAGAUUGCCGGGUACAUAAGGGC |
| env-3 | .AUUGACCAGUGAAGCCUAGUUGCCGCGAUGGGCUUGACUUCUCCAGCACGGGUAGCCUACAGUACCGAAUGAGGUAGUACUUUC |
| env-4 | .GAUCGGUCGACGUUCUCAUCGUGAUUCGCACUCCGCCUUCUCCGAUCUCCCCUCCACCCGAAACGGUCAAGCACCCAAUCGUUG |
| Ame-1-1 | GUGUAGAAAAGAGGGUUUCGUUGUAUAAUGUAGAACUGUGUAAUAGUAAUAAGUGUUGGAUAUUGUUAAUAGUUUGAUCAAGUGA |
| env-5 | AGUUGAUUAUCCCCACCCCGAGUUAAAUAGCUCAAAUUCGUUGACAGAAGCAUUCACGAGUUAUACAAUAAGCUCCUAAGACUAA |
| Tet-1-1 | .GCUAAUUAUUCUUUCAAAUUAUGGACAGGGGGAUUUUUCGACAAAAAUUUGGUAUACAGGUAUUUAAAAGAAGGAAUUACGCAU |
| Pth-1-1 | .ACAUUUGGCAUAUAUUGACACAAAAUAUAAAAACCGAAUAUAAUUAACGUAAAAUAUAAAAUAUUAAAAUAAAAGCAUUAAAUA |
| Dra-1-1 | .GUUGUGAGAAAUCUCAUAUUUGGAAGUGCUUCCUGAGCCAUUAUAAAGAAGUCCCCGCGCACCCCAUGCCUGGCCGUAUGAGGA |
| Dge-1-1 | .CAAAAGUCACUUUGCUUCCUUUUUGUGCUGCUGUUGUGACGAGCUGCUGUUGUGAGUUGGCAGUAAGGGUUCUGUUAGAGGUGC |
| Dra-1-2 | .UGGAAAGAAUCAGGGCGCAUUAAGACGCCUUAAGAGUUCUGUCAGAGAUCCCACUUUAGACUCGCUUCACUUCGCACAGAUUCA |
| env-6 | ..........................................................................NGGUUUUNNNG |
| Bsp-1-1 | .AAAUGAAUAAAAUAUAUAUUGACUUAAUAUGACAGGAAUCAUAUAUUUUAAUUGGCUUGCAAUUGUCUGUUUCGACAGAAAAUG |
| Dsp-1-1 | .AGGAUAUAGCAGUGGUAAUGUAUAGUAAUGAUAAUAUAGUAUCAGUUAUAUUGACUUUGCAUCUUUUGCAGGCCAAUAAUGGUA |
| Hor-1-1 | .GAUUUUUCUUGACUAUGGGAAAAAAGUAGGAUAUAAUAAAAUUGAAAUUUGUAUAAUUAUCCAAUAGUUUAAGGUAUUUAUUGU |
| Bha-1-1 | .GUUAUUCCUGUUAAAAGUAAUAACCUUAGACAAAAUUGUAUCAUUUUUAAGAAGUCGGACCAAAUUAAAUGCCUCGUGUAAUAU |
| Ckl-1-1 | .GUUAUUAGAGUGAUUUGAGUGAAAGUUUUAAUAUUUGGCAGUUAAAUUAUUUUCCAAAGUUUAUUGAAACUGGAGUGUUUUUAU |
| Cbu-1-1 | .UAAGUUAAAAAUGAUAUUAUAUAUAUGGAAAUAGAAAAAAAUGGUAUAUAAUUACAUUAUGUUAGGUUAUAUAUAAGUAAAAAU |
| env-7 | .UGCAUAUUUUAAUGCAAAUAAUUAUAUUUUUUAUCAAAAUAUGCUUGAAUAUAUGGAGUUAUUAGAAUAUUAUAUAUAUAAAUU |
| Cbe-1-1 | .AGGAAAUUAACAAAAAGAUAGUUAGAUUAUUGAUUUUUUAAUAUCUAUAUGAUAAUAUGACAUAUAUAUUAAAAAAAUGACAAU |
| Cbe-1-2 | .AAUAAAUAUUGUAAGGUAAUUAUUGCAUUUAAUGUUGAUUUUUCUUAUAUUAUUGUUAUAAUAUAUACAUAGAAAGACAUAAAG |
| Cbe-1-3 | .GGAAUGAAAUUCGACAAUUUAUAUUAAGAAUCAUUGAUUUUCUUACAUGAAUAUGAUAAUAUUUCUAUAUAUUAGGUAAGUGUA |
| Cbe-1-4 | .CUAAUUUAGUAUUCUUAAUGAUAUUAUUUGUUGAUUUUAUUAUAGAUUGAUGAUAAUAUUUCAUAUAUAUGAAAUAAGUACAUA |
| Cdi-1-1 | .UGUCUUUUAUUAGAUAAGUAUCUAGGAUACUAAUUUAUAUUUUAUUGAGUUGUUAAAAAAGUGCACUUAAAAAGUUAAAAAAGA |
| Cdi-2-1 | AUCUUAUAUCUAAAAAUAUGGAAAUAUUGACAAAAUAAUAUUACAAUGUUAGAAUAAAAAAGAAAUUUAUGAAAAUAUAUAAAAG |
| Cdi-1-2 | AUCUUAUAUCUAAGAAUAUGGAAAUAUUGACAAAAUAAUAUUACAAUGUUAGAAUAAAAAAGAAAUUUAUGAAAAUAUAUAAAAG |
| Cdi-2-2 | .AUGUAAAAAAGAUAGACAAUAGUAUAGAGGCAUAAUAUAAUAGAAGUAUAUAAUUGUAUUUUUAAUAUAAAUAAAGCAAUUUUA |
| Cdi-3-2 | .AUGUAAAAAAGAUAGACAAUAGUAUAGAGGCAUAAUAUAAUAGAAGUAUAUAAUUGUAUUUUUAAUAUAAAUAAAGCAAUUUUA |
| Cdi-1-3 | .AUGUAAAAAGGAUAGACAAUAGUAUAGAGGCAUAAUAUAAUAGAAGUAUAUAAUUGUAUUUUUAAUAUAAAUAAAGCAAUUUUA |
| Cpe-7-1 | .UUUGACAGGUUUUUUAAAAUACUGUAACAUACAAAUACAAAUUGAAAAUUAAGUGUUAAAAAGUAGUAAUUUUUAAGAAAUAAU |
| Cpe-1-1 | .UUUGACAGUUUUUUUAAAAUACUGUAACAUACAAAUACAAAUUGAAAAUUAAGUGUUAAAAAGUAAUGAAUUUUAAGAAAUAAU |
| Cpe-2-1 | .UUUGACAGUUUUUUUAAAAUACUGUAACAUACAAAUACAAAUUGAAAAUUAAGUGUUAAAAAGUAAUGAAUUUUAAGAAAUAAU |
| Cpe-8-1 | .UUUGACAGUUUUUUUAAAAUACUGUAACAUACAAAUACAAAUUGAAAAUUAAGUGUUAAAAAGUAAUGAAUUUUAAGAAAUAAU |
| Cpe-4-1 | .UUUGACAGUUUUUUUAAAAUACUGUAACAUACAAAUACAAAUUGAAAAUUAAGUGUUAAAAAGUAAUGAAUUUUAAGAAAUAAU |
| Cdi-1-4 | .UAUAUAAAUAGUAUAAACAGUGUUGCUAAAAUAUGGUAGAUAUUAUAUAAUAUAAGUGGAUAUGUAUAUACAUUAUGAGUUAUA |
| Cdi-2-3 | .UAUAUAAAUAGUAUAAACAGUGUUGCUAAAAUAUGGUAGAUAUUAUAUAAUAUAAGUGGAUAUGUAUAUACAUUAUGAGUUAUA |
| Cac-1-1 | .AUAAAAAAUAUUAAUUUUUUUUGAUUUACCUAUUAAAGAAAAUAAAAACUAUACGAAAAUAAUAUUGUUAAGAGUUUUAAAUAA |
| Cno-1-1 | ..AAUUCUUUAUUAUUAAUAAAAUAUAUAUUAAAAUUUUAUAUUGACAAAGUAAUAUGCAUAAAUUACUAUAAAUAUAAGAUUAA |
| Cte-1-1 | .AAAUGGUUAAAUUUAUUACCUAAUACUCAUUAUUGAUAAAAUCAUGAAAUUAAAGUAAUAUGUAUAUACAAUAGUUUAAUACCA |
| Cbe-1-5 | UAGACCAAUUAAUGAUCAUUAUGGUAUAAUUACUAUGGCAUAUAUUGGCUACAUUUAAUAUGUUAUGCAAGAAAUAAAAGAAAUA |
| Cbe-1-6 | .AUAUGUUAAAAUUUGAAAUUGUUUCAUUUUAUUCACUAUUUGUUCAUUGAUUUGUAAUUAAUUCCAUGUUAAAAUGAUCAUUAA |
| Cac-1-2 | .CAAUUUUAAGGUUUUAGCUAUGAAAAAGUAGUCUAGAGGCAGGUUGCUUUAAAUAUAAUUUUGUGGUAAAAUAUAACUAUUAAU |
| env-8 | 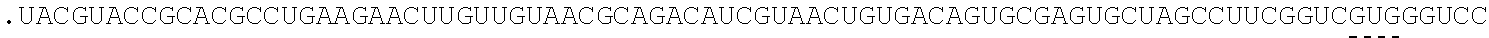 |
| env-9 | .UCUUCAAGAGGCCGAGGGAGCGACCAGCUGAAGCUGGCCGGACCAAGCGUUCAGGACGCANCUNGGCANCCGGGUUUNUGGGNG |
| Tca-1-1 | UACAUGAAUUAACAGUUCUUUGAAAUAUUGUAAACCCGUCUACUUCAUUGCACGAUUUUUACCAAUUUAUUACGAAUAUAUACGU |
| | ..................................................................................... |
| | ..................................................................................... |
| | ..................................................................................... |
| | -NNNNNNNNNNNNNNNNNNNNNNNNNNNNNNNNNNNNNNNNNNNNNNNNNNNNNNNNNNNNNNNNNNNNNNNNNNNNNNNNNNNN |

Alignment positions 86-170:

| | <-------------> <--------> <------> <----------------> |
|---|---|
| | 90 100 110 120 130 140 150 160 170 |
| Env-1 | UCAGAAUAGGUUGUUAGUUCGGGCGGC.GAAGAUCCGG..UGACUG..GCAUGGUC.GCUCC.....AUCCU..UUC...GGGAU |
| Env-2 | AAUUAUAACCUUCACCACUGUGGACGC.UUUGAUCCGG..UGUCUUG.AUUUGGUC.GCUUAA....CGUCUGGAACUUUUGACG |
| Env-3 | GUACGCCUUUGAAUCGAUUUCGGAUGC.CUUGAUCCGA..CCGCUGA.AUGUGGGC.ACUUAG...................... |
| Env-4 | 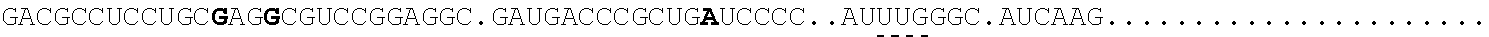 |
| Ame-1-1 | AGCAUUAAAGCGAAAAAUG.UAGAGCA.AAUGAACUGC..AGGUAU..ACAUGGAC.GCCUUA...................... |
| Env-5 | AAUGUGGGUUAAAUAAUUUGGUG.GGC.GUUGAUGUGC..CCUUUGU.AUCUGGUC.GCUUG....................... |
| Tet-1-1 | AAUUUGUAGAAUAUUUUAUAAAGAGUA.GAUGAAGUGA..GCUUAU..AUGUGGGC.ACUUUA...................... |
| Pth-1-1 | AAUCUGAUUUUAGAGAUAGUGUGAAGC.AGUGAUCUUG..CAGCUA..AUAUGGUC.ACUUUUUACG.................. |
| Dra-1-1 | 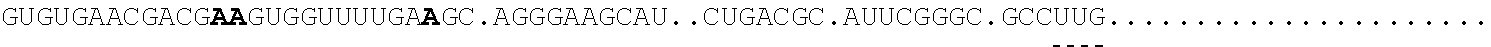 |
| Dge-1-1 | CUAGAUACCUUCUUCUUGUUCCGGGGC.GAAGAGGCGU..GUGGUGU.AUCCGGGC.ACCUG....................... |
| Dra-1-2 | CCCCAGGAACUUCUGUCGAAGAGACGC.GAUGAAUCCC..GCCCUGUAAUUCGGGC.ACCUC....................... |
| Env-6 | GAUUGUAUAGCCCGAUUCUGUGGAGGC.C.UGAUCCA...UAGCGGU.A.CUGGUC.GCAGC.....UCUUUCUGGAUAGAAAGA |
| Bsp-1-1 | CAAGGGUGUUUUAAGAAUAUAGAACAC.UGUGAUGAGC..GGUUUUU.AUUU..GC.ACUUUA...................... |
| Dsp-1-1 | UUGGAAUCAGCAAAAUUAAGGGGAAGC.GUUGAGCCGC..UACCCAU.AUGUGGUUCACUCG....................... |
| Hor-1-1 | AAAAUUUAAUAAUAUUGAAUGGGAAGC.UAUGAACUCU..UCUUUGU.ACGUGGUC.ACUUG....................... |
| Bha-1-1 | CGGAGCAUAAAAAAUCAAUAGGGAAGC.AACGAAGCAU..AGCCUUU.AUAUGGAC.ACUUG....................... |
| Ckl-1-1 | GCUAAAAUUAAACAAAUUGAGGGGAAC.GAUGAGGUAC..ACACUUUAAUUUGGGC.AUCCUU...................... |
| Cbu-1-1 | AAAAUAGAUUAUCAUGUAGUUGGAAAC.GAAGAAUAGU..UCUCUCA.AUCUGGGC.ACCUU....................... |
| Env-7 | CUAUGAAAACAUAAUAAAUCUGGAAGU.AAUGAUAAAU..UCUCUUU.AUUUGGGC.ACUUU....................... |
| Cbe-1-1 | UAUUAAUGAAAUAUAGUAAUUGAGAGC.UAUGAAGAAU..UCUUUUU.AUGUGGAU.AUUUU....................... |
| Cbe-1-2 | UAACAUAAAUAUACAAAGAUUGGGAAC.UAUGAAGAAU..UCUCUGU.AUUUGGGC.AUCUU....................... |
| Cbe-1-3 | UUUAUAAAAAAUAGAACAAUUGGAAGC.GAUGAAAAAU..UCUCUAU.AUCUGGGC.ACUUU....................... |
| Cbe-1-4 | UUAAUGUAUUAUAUUACAAUUGGAAGC.UAUGAAGAAU..UCUUUCU.AAGUGGGC.ACUUU....................... |
| Cdi-1-1 | AAAAUUAAAAAACCUAUUUAUAGAAAC.UGUGAAGUAU..AUCUUAA.ACCUGGGC.ACUUAAA..................... |
| Cdi-2-1 | AUUUUUAAUAAAAAUAUUU.UAGAAAC.UGAGAAGUAU..AUCUUAUUA.UUGGGC.AUCUGG...................... |
| Cdi-1-2 | AUUUUUAAUAAAAAUAUUU.UAGAAAC.UGAGAAGUAU..AUCUUAUUA.UUGGGC.AUCUGG...................... |
| Cdi-2-2 | AUUGUUUUUAUGAAAUAUUAUAGAGAU.GUUGAAGUAU..AUUCUAUUA.UUGGGC.ACCUUAU..................... |
| Cdi-3-2 | AUUGUUUUUAUGAAAUAUUAUAGAGAU.GUUGAAGUAU..AUUCUAUUA.UUGGGC.ACCUUAU..................... |
| Cdi-1-3 | AUUGUUUUUAUGAAAUAUUAUAGAGAU.GUUGAAGUAU..AUUCUAUUA.UUGGGC.ACCUUAU..................... |
| Cpe-7-1 | UAGUAAAUAUUAAUAUGUUAUAGAAAC.GUUGAUGUAU..AUUCUGU.AUUUGGGC.AUCUG....................... |
| Cpe-1-1 | UAGUAAAUAUUAAAAUGUUAUAGAAAC.GUUGAUGUAU..AUUCUGU.AUUUGGGC.AUCUG....................... |
| Cpe-2-1 | UAGUAAAUAUUAAAAUGUUAUAGAAAC.GUUGAUGUAU..AUUCUGU.AUUUGGGC.AUCUG....................... |
| Cpe-8-1 | UAGUAAAUAUUAAAAUGUUAUAGAAAC.GUUGAUGUAU..AUUCUGU.AUUUGGGC.AUCUG....................... |
| Cpe-4-1 | UAGUAAAUAUUAAAAUGUUAUAGAAAC.GUUGAUGUAU..AUUCUGU.AUUUGGGC.AUCUG....................... |
| Cdi-1-4 | UAAUUUUGAAUUUAAUAAAAUAGAAAC.GUUGAUUUAU..GUUCUGUAAUGUGGGC.ACCUUG...................... |
| Cdi-2-3 | UAAUUUUGAAUUUAAUAAAAUAGAAAC.GUUGAUUUAU..GUUCUGUAAUGUGGGC.ACCUUA...................... |
| Cac-1-1 | GGUAACAAAAAAUUAAGCGUUGGAGAC.UGUGAAGAAU..UUCUUGU.AUUUGGGC.ACCUU....................... |
| Cno-1-1 | CAAAAUAUUUACUUUUUAAAAGGAGACAUUUGAACUAU..AUACUUU.AUUUGGGC.ACUUU....................... |
| Cte-1-1 | UAUAAAUAUAAUAUUUUAAGAGGAAAU.UUUGAACUAU..AUACUU..AUUUGGGC.ACUUU....................... |
| Cbe-1-5 | ACAAGUAUAUUUUGUCAU.AAGGAAAC.UUUGAUCCAU..AUAUUUU.AAUUGGGC.ACUUU....................... |
| Cbe-1-6 | AUAGUAAUAUUAUGUAUAUAAGGAAAC.UGUGAUUCGA..AUAUUU..ACUUGGGC.ACUUU....................... |
| Cac-1-2 | AUAAAGAAUACCUAUUUGUUUGGAAAC.AAUGAUGAAU..UUCUUUA.AAUUGGGC.ACUUG....................... |
| Env-8 | CGCAUGUGGACCUCGCGAUUCGAGGGC.GAUGACGCUC..GUUCUGCAACAUGGGC.GCUUG....................... |
| Env-9 | GNAAAGCCGCCCUCACCCUUCAGAGGC.UUAGAUGCCU..ACCCCGCAACUUGGGC.GCUUG....................... |
| Tca-1-1 | AGUCAAAACAAUACUGCAA.UGGGUGU.GAUGAAGUCC..GGACAGUAAUGUGGGC.ACUUA....................... |
| | ............<<<<<<<<<<<.<<<.......<<<<..<<<<.......................<<<<<<<....>>>>>>> |
| | ............????????222.222.......2222..2222.......................2222?22....22?2222 |
| | ....................................................<<<<.<<<......................... |
| | ....................................................0121.222......................... |
| | NNNNNNNNNNNNNNNNNNNNNRGARRC-NNUGANNYRY--NNNYUNN-AYNUGGRC-ACYUN----------------------- |

Alignment positions 171-255:

| | 180 190 200 210 220 230 240 250 |
|---|---|
| | <-------> <-> <-------> <---------> |
| env-1 | ACGGUCACUGA.GGAGCCAGUAGCGAAACCGGCCCUA..UGGGUCGGUUUGUUUGUUUAAAGGAACCCGAAGGAGGCCGUUCUUC |
| env-2 | AG.GGCGCCGG.GGAGCGAGUAGCGAAACCGGCCAC..UGGUACUGCGGCCGGUUUUGUCUGUUUAAACAGCCCGGAGGCGCCGC |
| env-3 | ...GCGGCCGG.GGAGCAGGUAGUGCAACCGACCGUUCCGCACCGGAGUGUGUCGGUUGUGUGUAUUUCGUCUCAGAAGAGGGCA |
| env-4 | 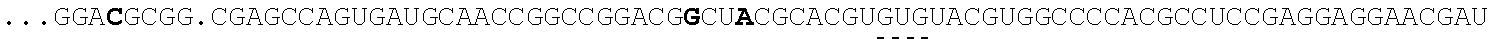 |
| Ame-1-1 | ...AACUGCAG.GGAUGUAGUGGCGUAACCGACUAACAAUAUUCAAUUAGAUUGUUAGUCUCUAUUUUUUUGAAUAUAAAUCGAU |
| Env-5 | ...AGGGGUAC.CGAGCCAAUAGCGAAACCGCCGCCGUCAUAGAGGGGAGCACUAUGAUCGCUAGUCGGUUGCGCAAAAUAACAA |
| Tet-1-1 | 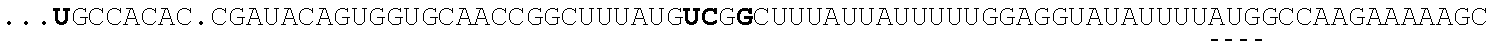 |
| Pth-1-1 | ...GCUGCAAG.UGAGCUAAUAGUGAAACCGGCACACUGAUGCCCGGUUUUUUUGUUUAUUAAUAAUGGCAGAUGAUGGUUAGGC |
| Dra-1-1 | ...UCAGAUGU.GGAGCGAGUGGCGCGACCGGCAAAGCUGUGGCAACACACUUGCCGGUUGUUUGUUGUCUCUCGGCUGGCCCCG |
| Dge-1-1 | ...CCACACGC.GGAGCCAGUGGUGCGACCGACG.AACCGUGAACCCUUUGGGGAUCGCGUUCGUGGGUCGCUUUUUGCUGCCUA |
| Dra-1-2 | ...GGACGGGA.GGAGCAAGUGGUGCGACCGGCUUUUCGUUGGUGUCUGGCCCCGGAGCUGUUCCAGGCGUCUGGCCCUUAUUUC |
| Env-6 | AC.GCUAUUGG.GGAGCCAGUUGCGAAACCGGCCACGUAGCCGGUUUUUGCGUCUUAAGUGCCUGACGGGGGUCAGAAAAGCUGG |
| Bsp-1-1 | 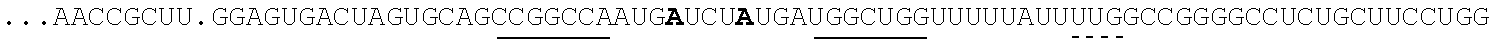 |
| Dsp-1-1 | ...GAUAGCGG.GGAGCUAAUAGUGAAACCGGCCCUUUAGGGGUCGGUUUUGUUUUUGGUCAAAUUAUAGAGAUGCUUAUAAGGA |
| Hor-1-1 | ...AAGGAGAG.GGAGCUAGUAGUGAAACCGCCCCGACCGGGGGCGGUUUUUGCUUUUUACAUACCUUUAAAAUAUUUCAGGGGG |
| Bha-1-1 | 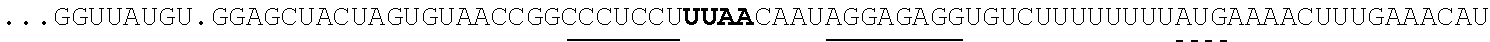 |
| Ckl-1-1 | ...GUGUGUAUUGGAGUUAGUGAUGCAACCGACCCUGUAUUCAUAAAUAUUUUAUGGAUACAGGGUUUUGCUUUUUAAAAAUGGA |
| Cbu-1-1 | ...GGGAACUA.GGAGUUAGUGGUGCAACCCGCCAGCAAAUUAAUUAGUAUUAAUUUGUUGGUUUUUUUUAUAAAAAAUAAGAGG |
| Env-7 | ...GAGGAUUU.UGAACUAGUAGUGCAACCCGCCAACAAUUAAUCAAGAUUGUUGGCGUUUUUGAUGAUAGAAAAUAUGUGAAAA |
| Cbe-1-1 | ...AAGAAUUU.CGAGCUAUCUGUGCAGUCGACCAAUUUAAACUAAUAAGUUCUUUUUUGAUUAGUUUAAAUUGGCCUUUAUUAU |
| Cbe-1-2 | ...GAGAAUUU.AGAGUUAAUGAUGCAACCCACCAAUCAAAACUAAUUAAUUUUUUUAAUUAGUUUUGAUUGGUUUUUUAUAUUU |
| Cbe-1-3 | ...GAGAAUUU.GGAGCUAGUUGUGCAACCGACCAAUUAAAACUUAGUAAUCCAUAAAUAUAUUAAGUUUUUGUUGGUUUUUUAU |
| Cbe-1-4 | ...AAGAGUUU.CGAGCUAGUUGUGCAACCGACCAAUUAAGAUUAAUUAAUUAUUUUAUUAGUUAGUUUUAAUUGGUUUUUUAUG |
| Cdi-1-1 | ...AGAUAUAU.GGAGUUAGUAGUGCAACCUGCUAUAAAUAUAAAUAGGAGUAACUUUUAAUUGUCAAAAAUAUAUAAAAUAAAU |
| Cdi-2-1 | ...AGAUAUAU.GGAGUUAGUGGUGCAACCGGCUAUGAAUAUAAUUUUUAUAUUGCAUAGUCUUUUUUUGCUUUAAAAUUAACAA |
| Cdi-1-2 | ...AGAUAUAU.GGAGUUAGUGGUGCAACCGGCUAUGAAUAUAAUUUUUAUAUUGCAUAGUCUUUUUUUGUUUUAAAAUUAACAA |
| Cdi-2-2 | ...GGAUAUAC.UGAGUCAGUGGUGCAACCGGCUAUGAAUAUAAAUUUAUUUAUUUUCAUAGCUUUUUUUUAUAUAAUUUUUUAG |
| Cdi-3-2 | ...GGAUAUAC.UGAGUCAGUGGUGCAACCGGCUAUGAAUAUAAAUUUAUUUAUUUUCAUAGCUUUUUUUUAUAUAAUUUUUUAG |
| Cdi-1-3 | ...GGAUAUAC.UGAGUCAGUGGUGCAACCGGCUAUGAAUAUAAAUUUAUUUAUUUUCAUAGCUUUUUUUUAUAUAAUUUUUUAG |
| Cpe-7-1 | ...GAAUAUGC.UGAGUUAGUGAUGCAACCGACUAUAUUUAUAAAAAGUUAUAAAUAUAGUCGGUUUUUUAUUUUCCAUUUUUUA |
| Cpe-1-1 | ...GAAUAUGC.UGAGUUAGUGAUGCAACCGACUAUAUUUAUAAAAAGUUAUAAAUAUAGUCGGUUUUUUAUUUUCCAUUUUUUA |
| Cpe-2-1 | ...GAAUAUGC.UGAGUUAGUGAUGCAACCGACUAUAUUUAUAAAAAGUUAUAAAUAUAGUCGGUUUUUUAUUUUCCAUUUUUUA |
| Cpe-8-1 | ...GAAUAUGC.UGAGUUAGUGAUGCAACCGACUAUAUUUAUAAAAAGUUAUAAAUAUAGUCGGUUUUUUAUUUUCCAUUUUUUA |
| Cpe-4-1 | ...GAAUAUGC.UGAGUUAGUGAUGCAACCGACUAUAUUUAUAAAAAGUUAUAAAUAUAGUCGGUUUUUUAUUUUCCAUUUUUUA |
| Cdi-1-4 | ...GAGCAUAU.UGAGUUAGUGGUGCAACCGGCUAUGAAAUUGUAUUUAUUUAUAGAUACUAUUAUUUUCAUAGCUUUUUUUAUU |
| Cdi-2-3 | ...GAGCAUAU.UGAGUUAGUGGUGCAACCGGCUAUGAAAUUGUAUUUAUUUAUAGAUACUAUUAUUUUCAUAGCUUUUUUUAUU |
| Cac-1-1 | 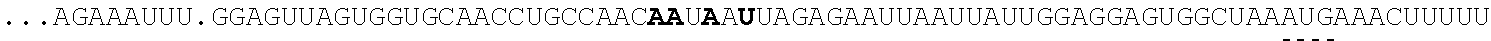 |
| Cno-1-1 | ...GUAUAUAG.GGAGUCAUUAGUGCAACCGACCUUAUUUUAUUUAGGGUCGGUUUUUAUUUUUUUUAGUAAUAUGGUUAAUAUU |
| Cte-1-1 | ...GUAUAUAG.GGAGUUAGUAGUGCAACCGACCUUGAUUAAUCAGGGUCGGUUUUUAUUUUUAAAUAAAGCAAGAAAUAACUAG |
| Cbe-1-5 | ...AUAUAUGG.UGAGUUAGUAGUGCAACCGACCUUUAUAUUUUAUAAAGGCCGGUUUUUAUAUUUUUUAAAUAAAAGUAUGAAA |
| Cbe-1-6 | ...AUAUUCGA.UGAGUUAAUAGUGCAACCGACCUUUUUAUUAAGGUCGGUCUUUUUUCGUCCGCUUAAAUAAGUAUAUAGAUAA |
| Cac-1-2 | ...AGAAAUUU.UGAGUUAGUAGUGCAACCGACCAA.CGAUUAAUUAAGCUGUAUUAAUUGUUGGUUUUUUGCUUGUGUAAGGAG |
| Env-8 | ...GAACGAGC.CGAGCCAGUAGCGCAACCGAUCGG.UCGACGUUCUCAUCGUGAUUCGCACUCCGCCUUCUCCGAUCUCCCCUC |
| Env-9 | ...GGGUACGC.GGAGCCAGUAGCGCAACCGGCCGAGGGCUUCCUUUUGCUUCCUCACAUCCCAGGCUUUGAGGGUCCCUUAGGU |
| Tca-1-1 | ...GUCCGGAC.CGAGCAAGUAGUGCAACCGACCAGAUGCAAAAUAACGUUUUUGCUGUUGGCGGUUGUUUUUCGUUUUUGGGGG |
| | ...>>>>>>>>....>>>..............>>>>>>>>>>>.......................................... |
| | ...22222222....222..............222????????.......................................... |
| | .....................>>>>>..>>....................................................... |
| | .....................22212..10....................................................... |
| | ---RNNNRYRN-NGAGYYAGURGUGCAACCGRCYNNNNNNNNNNNNNNNNNNNNNYNNNNNNNNNNYNNNNNNNNNNNNNNNNNN |

Alignment positions 256-340:

| | 260 270 280 290 300 310 320 330 340 |
|---|---|
| env-1 | CCAACACGCGUGACUCCUUUUCCACUUAUCUUUUCCAUUCGAAGACGAUUAGACCAGGAGGACCUAUGAUUUCAAGACGAGACUU |
| env-2 | GGGCGUAAAUUCGACCGAAAAUCGACUGGAACCGCUCGAUCCAGACGGGGGAUCGUUCUAUAAUUGGAGCAGCGGGGUGUCCCUG |
| env-3 | CCUUCUGAUGGUUAGAGAGCUGAUCAAGGGCUGGUAGCCCUUCCGAGGAGAAUUCGGCAUGCGAGCGCACUUCAUGCGCGUUGGC |
| env-4 | GCAAGACCUCGCACGCCCGCGGAGCGGCCUGCUCCGCAACCUGCCGCGCACGAUCCUGUUCGCGCUCGGCCUCAUCGGAUUACUC |
| Ame-1-1 | 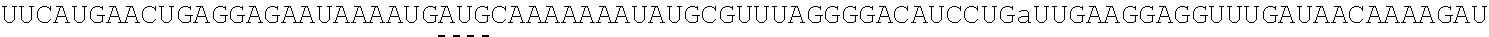 |
| Env-5 | AAAAGAUUAAGAACUCGGUUGAAAUGGGCUUGGCGGCAGUACUGCUACUGAGUCAGUUUGCUUUUACAUUUGGCACGUUAAUUAA |
| Tet-1-1 | UAGGGGAGCUUUUACUUGAGGUAGGGCUUAUAACAGAAGAACAAUUAAAGCACGCAAUAGAAGUUCAAAACAAAACAGGAGAAAA |
| Pth-1-1 | AGUUAUUAAAUUCUAAGGGGGGUGAAAAUAUUUAUAGAUAACAAACUGUACCGCAUUACAGCACUGCCAUGACCGGAUGCCUUGC |
| Dra-1-1 | 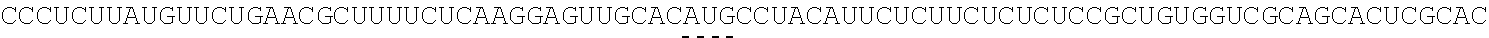 |
| Dge-1-1 | CCUCUCGGCCCCGCUGUUUUCAACGCUCGACCCUUUUUCUGCUCUCCAGCGAUGGUGCUUCCGCCGCUGUGGGAAGCGCUUCUAU |
| Dra-1-2 | GCGGCCCCUGCCGCUUUCAGGAGUUCUCAUGUUCAAUACCAGAAAGUCUGCCCUCGUCAUCGCCGCCCUGCUCAGCCUCGGGGCC |
| Env-6 | UCUAACCUCUUCGAAGCCAAUUCUGCUGUCACAUCCUUGCCUUCAACGCUCCCCUCAGAAGUCGCUGCGCUUCUCUUUGUUUACU |
| Bsp-1-1 | CUGAUGCUUAGGAUGAAAGAGGUGCAUUUAAUGAAGCUUGAUAAAUACAAAACUUUAUUGUUUCAAAAGAUUAAGAAUCAAAUGA |
| Dsp-1-1 | 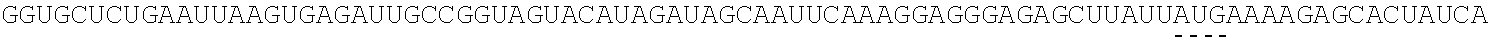 |
| Hor-1-1 | GGGAUUUUUAAUGAAAAAAGGUUUAAUUUUUGGACUGGCUGUAGUGUUGACAAUGGUGCUUUCUUUAGGCGUUAUGGCAGCAGGU |
| Bha-1-1 | AUCAGCUACAAAUUACGGAUAUUUGCAUUGAUUGCAUUACUCAUUUCACAAACGUUGCUUACGAGCUUAAGUCUUCCAUUUCAAG |
| Ckl-1-1 | 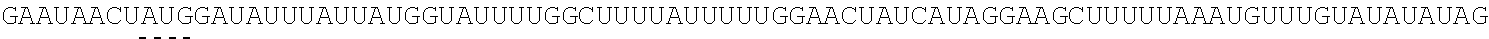 |
| Cbu-1-1 | AAAAAAUAGAUUAUUAUGAGAUUUAUGUAGAUAUAGGAGGAUUUAUAAACUUGGAUUAUAUAUUAGUAAUAAUAUUAGGAUUGGU |
| Env-7 | UUUAUGAAUAAGGAAAUAUAUACUAGAUUUUAAUUAUACAUAAGUAUUUAUUUAUAUAUUAAUGAAAUUACUGAUAUAUAUGUUC |
| Cbe-1-1 | 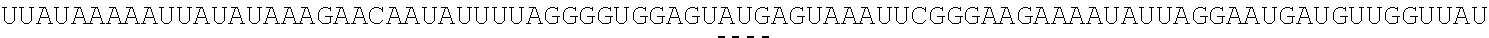 |
| Cbe-1-2 | 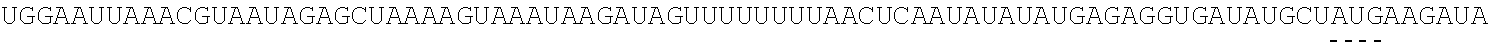 |
| Cbe-1-3 | 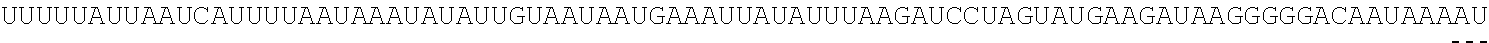 |
| Cbe-1-4 | UGAAAAAAUUUACUUGAUUUAUAUAUAAAUAAGAACUUAUAUAUCUUAAGUUUAAGCAUUACGUAAGUAAGCUCAUUGAGAGAAC |
| Cdi-1-1 | ACAUAAAUAAGUAAUUAAUAGAAGAUAGAUAUAAAUAUAUUUUUGAAAUGCUCCCUGCAUUCAUAACGAGUGUAGAGUGGCAGAU |
| Cdi-2-1 | AAAUGUUGCAAAAUUUGAAAAUAAGAGUAUUAGUCGUUAAGAUUUUUAUUGAUAGGUGAAAUUUUGGCUUUUAAAGUAGCCAUUU |
| Cdi-1-2 | AAAUGUUGCAAAAUUUGAAAAUAAGAGUAUUAGUCGUUAAGAUUUUUAUUGAUAGGUAAAAUUUUGGCUUUUAAAGUAGCCAUUU |
| Cdi-2-2 | 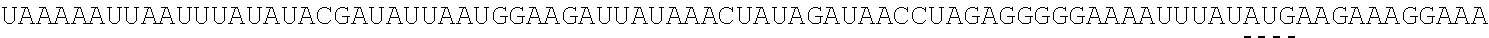 |
| Cdi-3-2 | AAAAAUUAAUUUAUAUACGAUAUUAAUGGAAGAUUAUAAACUAUAGAUAACCUAGAGGGGGAAAAUUUAUAUGAAGAAAGGAAAU |
| Cdi-1-3 | 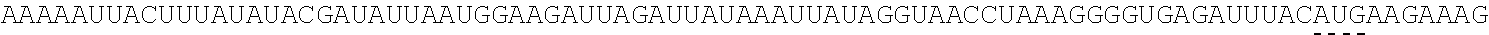 |
| Cpe-7-1 | 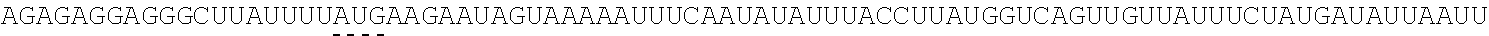 |
| Cpe-1-1 | 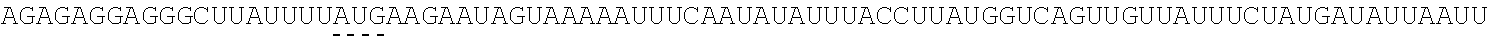 |
| Cpe-2-1 | AGAGAGGAGGGCUUAUUUUAUGAAGAAUAGUAAAAAUUUCAAUAUAUUUACCUUAUGGUCAGUUGUUAUUUCUAUGAUAGUAAUU |
| Cpe-8-1 | 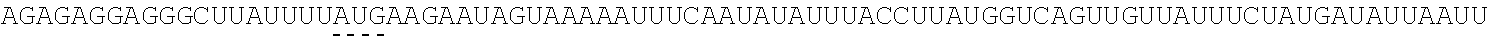 |
| Cpe-4-1 | AGAGAGGAGGGCUUaUUUUAUGAAGAAUAGUAAAAAUUUUAAUAUAUUUACCUUAUGGUCAGUUGUUAUUUCUAUGAUAGUAAUU |
| Cdi-1-4 | UAAAAAAUGGUUAUAUGUUAGAAAUAAAUUUGGUUAGUACGUUUAAAUAAUUUUACUUAGAUUUUUAACCAGAUAUAUUAUAAGU |
| Cdi-2-3 | UAAAAAAUGGUUAUGUGUUAGAAAUAAAUUUGGUUAGUACGUUUAAAUAAUUUUACUUAGAUUUUUAACCAGAUAUAUUAUAAGU |
| Cac-1-1 | AAAACCCUUAUUCUUAAAUCAAAUUUGAAAAGACUAGGAGGUGGGCACUUAGGAACCCAAAAUUUUGAUGUUUUUUCUAAGUUUA |
| Cno-1-1 | 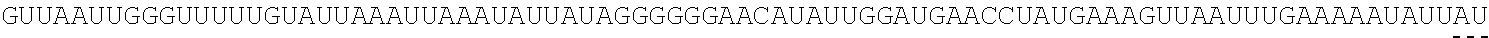 |
| Cte-1-1 | 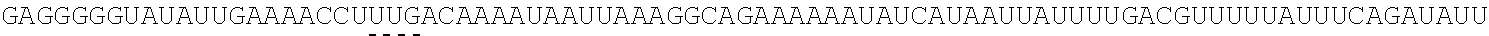 |
| Cbe-1-5 | UAUUUUUAGGUAAGACAGAGGAAGAUAUUGAAAUACAAUCAGAUUAACUGUAUUUUAAUAAAAAGAAAGUUUAUUAUGUUUAUAU |
| Cbe-1-6 | 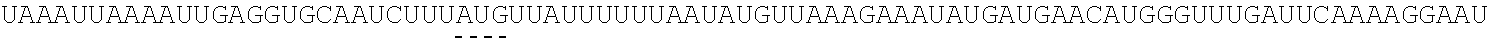 |
| Cac-1-2 | GUGUUUAAACUAUUAAAGUAUAAAAAUGUAAUUUAUGUAUUAUUGUUCAAUAUAAUUUACAUAAUAGGGAUAUAAGAUGUAGAGA |
| Env-8 | 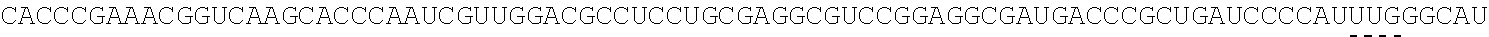 |
| Env-9 | AGUCAUGGCAGCCCCAUGCCUACCGGAAUGCGAGGAGUACGUGAGGAACGCGAACAACGCACAUGGCACGUGAGGACGGCUCAGG |
| Tca-1-1 | 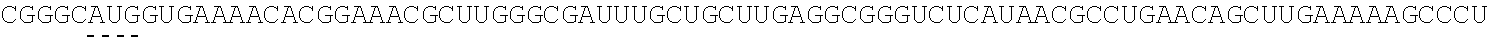 |
| | ..................................................................................... |
| | ..................................................................................... |
| | ..................................................................................... |
| | ..................................................................................... |
| | NNNNNNNNNNNNNNNNNNNNNNNNNNNNNNNNNNNNNNNNNNNNNNNNNNNNNNNNNNNNNNNNNNNNNNNNNNNNNNNNNNNNN |

Alignment positions 341-425:

| | 350 360 370 380 390 400 410 420 |
|---|---|
| env-1 | 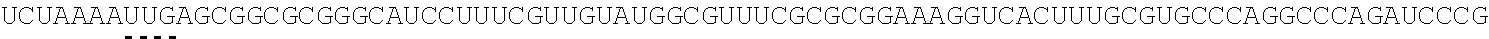 |
| env-2 | 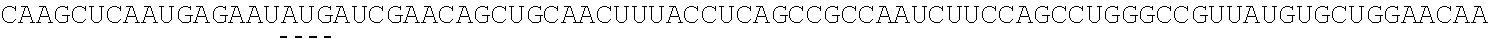 |
| env-3 | GCGCUGAUGUCAGUCCUCGGCACGCUGGCUGCCCUGGCCCUGGCGGGGGGAGCCGGCGUUCGAGGCUGGUAGGGGGCCGUGGUAG |
| env-4 | GUCACGGGAUGUUCCACGUCCGCCUUGCCCAGCGAGACCCUCGCCGACCCCGUCAUCGAGUUCACGAUCCCCGCGACCCUCACCC |
| Ame-1-1 | CAACUUAAUGAGAUACUGAAFFUUCAAAAAAUAAAGGGGGGGGGGUUAGGAGAGCUUAUCGUUGAUGAAGGGAUUUUAAAAGAAA |
| Env-5 | CGCGACAUCGGUAUACGCCGCAAACGAUAUAACUGCUUCCAACCACGAAGGCCAGCUCUCCCCUGCAGGCACUUGGUCCAAUGGC |
| Tet-1-1 | GUUAGGAAAAGUUCUUAUUAAAUUGGGGUAUGUUACAGAAAGCCAAAUACUAGAGGCUUUAGAAUUUCAAUUAGGUAUUCCACAU |
| Pth-1-1 | 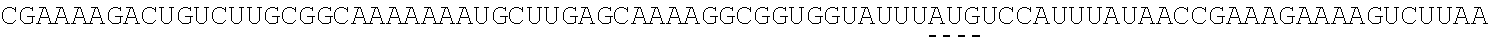 |
| Dra-1-1 | UGGGUACUGCUGGGGCACAGACCCUCGCUACUCCCCCCACUACUCCCGCGGGCACGGAGAUCAUCAACAAGGCGGAAAUCACCUU |
| Dge-1-1 | 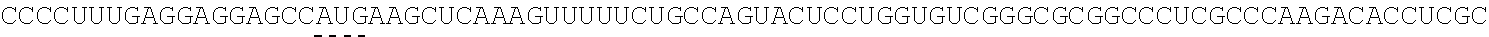 |
| Dra-1-2 | CCCGCCCUGGCGGGGACGAGCAGCACCCAGACCGACUACGACAACGCUACCCUGAGCCUGAGCGCCGACGGCACCUAUGCCCAGG |
| Env-6 | UCGGAUCUGUCAGUGAACGAGAAAGGAGACGAAAGCACACCAUCUCUCAACCAUCUUCAAGCAUGUUCGAACCCACAACAAGUUC |
| Bsp-1-1 | CUCUUUGGUUUGAACAGGAAGCGGAAACUCCUGUGAACAACGAAGAUGUUUACCGUUUCCUUCACUCCAUCAAGGGGACAGCAGG |
| Dsp-1-1 | AUAUUGCUUUCACUUUCAAUUCUGGUAAGCUUGGGAUUGUUUUCCGCUGUGCCUGUAAGCGCGGCGAUAGUAAAUGUAUCUAUCA |
| Hor-1-1 | AAUGAUUCGGGCACUGUAAAUGUAACUGCUUCUGUGCAAA............................................. |
| Bha-1-1 | CAGUAGCCAAUUCCGAAAAGAGUGGACUAACGAAUGUGGAAAUAACCGAUGCGAAUGGACAAACAAUUGAUGCAGAUCAAUUUCC |
| Ckl-1-1 | AAUACCUAAAGGUGAGUCUAUUUUACAUCCACCUUCUCAUUGUAUGAAUUGUAAUAAAAAAAUCAAGGCCUAUGAUAUGAUUCCU |
| Cbu-1-1 | UAUAGGAAGUUUUUUAAAUGUUUGUAUUUAUAGAAUUCCAAGAGAAGAAUCAAUUUCGUAUCCACCAUCUCAUUGUGGAAACUGC |
| Env-7 | UAAUUAAGAAUCUUCAUUAAAAUCUGCCCUAAACCCAAAAAGGAUAUAUGGCUCAUAUGAAGUAAUUUUAUUGGAACUUAUAUAA |
| Cbe-1-1 | UUUAUCAUCUAUAGCUAUAAUAUUAGUUUCAGUAAAUCUGAUUAUGUUUGAAAAAUUUAAAAAGGAACUUAGGCAAACAGUUACU |
| Cbe-1-2 | CAUAAUGAAAUCAUGAAAGUUAUAAAUGACAAUUUGGAAAAGUGUUCUAAAUUCGAAUUUGUUGCAGAGUUAAGAGAUUUAACUU |
| Cbe-1-3 | 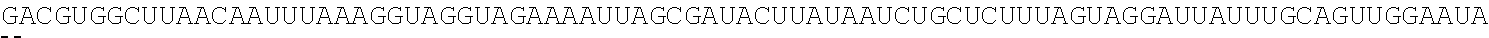 |
| Cbe-1-4 | AUUUUAAUUUUUAUCAAUCACAAUUAUACUACAGCUACAGUAAGGGUUAAAUUAAGAGGUAUAUAAGUAUUAAUGAAGGGGAUGA |
| Cdi-1-1 | AUCUGCCCAAAACUCCUUUAAUUGCUGGAAGUUCCUAAAGCUAACUAAACCACAACAUAAUCAUGAAAUAAGGAUAAGUGUGAUA |
| Cdi-2-1 | GUUGGAAAAGGGAAAUUUUUUAAAAAGUAUAAUACUAUUAUAAUAAGAAAAUAAAGUUUUUAUAAACAUUGAGAUGCAAAAUAUA |
| Cdi-1-2 | GUUGGAAAAGGGAAAUUUUUUUAAAAGAAUAAUAGUAUUAUAAUAAGAAAAUAAAGUUUUUAUAAACAUUGAGAUGCAGAAUAUA |
| Cdi-2-2 | UAAAAAGGCAUUAUUAAUUAGUUUAAUUAUGAUUUUAAGUAUGGUGGUGUCUACAAUAUAUCCAACUGUAUCUUAUGCUUCAGAA |
| Cdi-3-2 | AAAAAGGCAUUAUUAAUUAGUUUAAUUAUGAUUUUAAGUAUGGUGGUGUCUACAAUAUAUCCAACUGUAUCUUAUGCUUCAGAAU |
| Cdi-1-3 | GAAAUAGAAAGGCAUUAUUAAUUAGUUUAAUUAUGAUUUUAAGUAUGGUGGUGUCUACAAUAUAUCCAACUGUAUCUUAUGCUUC |
| Cpe-7-1 | AUGAUUUCUCCUGGAAGCAUUGUUUUAGGAGAAGAAACAAAAUCAGUUCAAAAUGAUGGGGCUGUUGAAAUUACAAGCACUACCU |
| Cpe-1-1 | AUGAUCUUCCCUGGAAGCAUUGUUUUAGGAGAAGAAUCAAAAUCAGUUCAAAAUGAUGGGGCUGUUGAAAUUACAAGCACUACUU |
| Cpe-2-1 | AUGAUCUUCCCUGGAAGCAUUGUUUUAGGAAAAGAAGCAAAAUCAGUUCAAAAUGAUGGGGCUGUUGAAAUUACAAGCACUACCU |
| Cpe-8-1 | AUGAUUUCUCCUGGAAGCAUUGUUUUAGGAGAAGAAACAAAAUCAGUUCAAAAUGAUGGGGCUGUUGAAAUUACAAGCACUACCU |
| Cpe-4-1 | AUGAUCUUUCCUGGAAGCAUUGUUUUAGGAGAAGAAACAAAAUCAGUUCAAAAUGAUGGAGCUGUUGAAAUUACAAGCACUACUU |
| Cdi-1-4 | 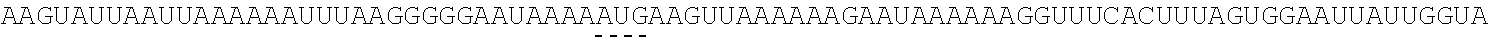 |
| Cdi-2-3 | 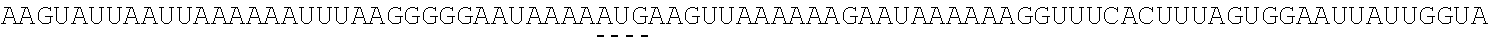 |
| Cac-1-1 | UAGGUUUAUGUCUAUCUUUAAAAUAUUUCAUAUUAAAAUAUAAGAGCAAAUUUUAUAAUGAAGAUAAGAUAAAUAAAUUAGAUGG |
| Cno-1-1 | 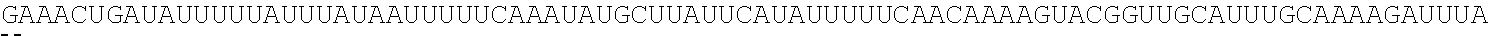 |
| Cte-1-1 | AUUAUGCUCAAAAACUGUAUUAGCAGCAGAUGUAGAUGUUAUAGAUAAUGUUAAAAUAACUAAUGAAAAGGGGGUAGUGCAAGGU |
| Cbe-1-5 | AAUUCCUAUUAAAUAUUUAAAAAAAUGUAGUUAAAAUACAAAUUUGUACAUAACAACGUUUACAAAAAAGUUAAAUAAUAUUAAA |
| Cbe-1-6 | UCAUAAAAACGGUACAAAAUUUAAUGAAGAGGGUUUUGAUAAAAAAGGGGUUCAUAAAAAUGGCACAUAUUUUAAUAGAGAAGGC |
| Cac-1-2 | GGUUAAAUAAAAUAAUAGUUUAUAAAUGCCAAGCAUCAUAUUUUAAUAUUAAAAAGAUAUAAUGUUAAUUCGUUUUUCAACUAUA |
| Env-8 | 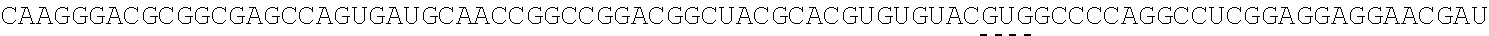 |
| Env-9 | CGUUCGAGGAGCCUGCUGGCAGUGCUAUCCGCCCUGGUGCUGGUACUAGCGGCCUGUUCGACGCCAGCCGAAGAACCGAUGCCGC |
| Tca-1-1 | |
| | ..................................................................................... |
| | ..................................................................................... |
| | ..................................................................................... |
| | ..................................................................................... |
| | NNNNNNNNNNNNNNNNNNNNNNNNNNNNNNNNNNNNNNNNNNNNNNNNNNNNNNNNNNNNNNNNNNNNNNNNNNNNNNNNNNNNN |

Alignment positions 426-509:

| | 430 440 450 460 470 480 490 500 509 |
|---|---|
| env-1 | GGCGGCACACUCAACCCCCAUGACGUGCCCAAAUAUAUGACGCCCCUGCUCAUUCCGCCGGUCAUGCCCAAGGCCGGAACGAUC |
| env-2 | AGCAUAUAUGCGCUGGCGGGUUGGAUCCCGUCAAUUGUUGGCAUUGCCGUGCGCGGCCUGCUCUACCGUCUGAUCCUGCACAUG |
| env-3 | ACUCCGGCCGCGCUUGACGCUUAGCGAUUGCCGGUGNGUCCAACGUUGCCCGCCUAGUCCUCAUUACAGUAGCCGCCGCAGU.. |
| env-4 | CAACCUUCGAAGACACGCAGGUGCAACGGUUGGACGGCACGACGCUCGAGAGUGACGUGAGCGUCUUCGUUCGCGAUCACCG.. |
| Ame-1-1 | GUCAGAUUCUAGAGGUUUUAGAAUUUCAAUUGGGCAUACCACAUAUUGAUUUAAAUAAAUAUCAUAUUAAUCCUAAGGCAGU.. |
| Env-5 | A................................................................................... |
| Tet-1-1 | GUAGAUUUGCAAAAAUACUAUAUUGAUCCCGAUGUAGCAAAAUUAAUUCCUGAAGCCGUUGCUAAAAGACAUACUAUUAUUC.. |
| Pth-1-1 | GGCCUUAUUCAGAAUAAGUUUACUUGCUGUUUUACUGUUGUGCCUCAUUCCGUUACACAAAGCGUCAGCCGCUUCCGGCGAA.. |
| Dra-1-1 | CACGCCCGAAUCCACCCCGACCAACCCCAACCCCCCCAGCGAAACCAUUGAGACGCCGCCCGUCAUCACGGUGGUCAACCCG.. |
| Dge-1-1 | CUCUCCAGCUGGUCUACGACCAGGUGCUGAUCCAGACCGUCACGCAGAACGGCAAAGCCACCGAGAAACGCACGCCGGGCGUG. |
| Dra-1-2 | CCGUCAGCUUCAGCGUGCCCAGCACCACCGUCACGCUCAGCGCCGAGCAGAUUCGCCCCGGCAACAGCUUCACGCUCUCCAU.. |
| Env-6 | UAUUCGAGAUAUUGGAUGAAUUUGCCUAAGGAGAAACAGAUGCUUCACAGAAUAUCGAUCGUAUUUCGAGCCCUGCUGAUCG.. |
| Bsp-1-1 | AACUCUGCAGCUUGUCGGACUCCAUCAGGUGGCAGGCAAACUGAUGGACCAGGUGGAAAAGAGCAGCGAAAAAAUCUGGGGA.. |
| Dsp-1-1 | GCAGCGGCACUUCAACUGAGAUUGUAGGCGUAUACAAUAAGGCUGGAGGGGGAUCAGUAUACGUUGACCUAAGCGGUUCACC.. |
| Hor-1-1 | .................................................................................... |
| Bha-1-1 | AGACCGUUUCGUUUCGAUCGAUUCGAUUUUACAAAUCAUCCUUUCAUGGAGCGUUGACGAAAGUGUAGCACAGGGGGCGGAG.. |
| Ckl-1-1 | GUAAUAACUUAUAUCAUUUUGAGAGGAAGAUGCAGAUAUUGCGGCAAAAAGAUACAUUUAAGGUAUCCUGUCUUAGAAUUUA.. |
| Cbu-1-1 | GGUCAUAAUCUACAGCCUGUAGAUUUAAUUCCUAUAAUAAGUUAUGUGUUUUUAAAAGGAAAAUGCAGAUAUUGUAAGGAGA.. |
| Env-7 | AUACAAAUAAACUUAUUAUAAAAAUUAAAUAAAUUAUUAAAAGGGGUUGAUAUAUUGAUUAUAAGGCGGUUAAGUAUAGGCC.. |
| Cbe-1-1 | CAAUGUAUUUCAGAUUUAACUGUUUCCAUUGAUGGAGACAAGCUUGAAAAGCUUAUAAAAGAGCAAUCAGAUGACAGUGUAG.. |
| Cbe-1-2 | UAGCAGAUAUGUAUUAUAUUGAAAAGAUAUCGAGUAUUGAUAGUAUAAAAGCGAAGUUCAAUUAUAAAAUAAUAAAUAACAC.. |
| Cbe-1-3 | ACCGGUUACUAUUUUUUACAAACGUCUAGUAAAAGUAUGGAGGUUAUGUAUAGUGAAAGAUUGCUAUCAAGUGAAUGGCUUA.. |
| Cbe-1-4 | 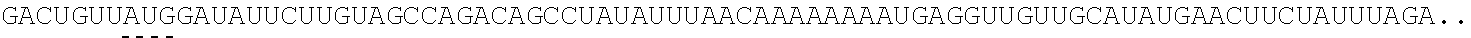 |
| Cdi-1-1 | 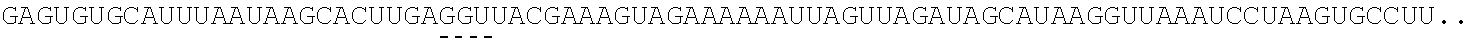 |
| Cdi-2-1 | AACCUUUUGUAACAAUAUAUUUACCAAAAUAAUACAAUUUAUUUUAUAUUAUAACCAUAUAAAUCAUAUAAAUCAAUACUCA.. |
| Cdi-1-2 | AACCUUUUGUAACAAUAUAUUUACCAAAAUAAUACAAUUUAUUUUAUAUUAUAACCAUAUAAAUCAUAUAAAUCAAUACUCA.. |
| Cdi-2-2 | UUAGGAGAGAAUAGUCAGAUUCAAAGUGGUUCAACUAAUUCAUCAACUGGAGAGGAGAAGGAAAGCGACAAUAAAAAACCAG.. |
| Cdi-3-2 | UAGGAGAGAAUAGUCAGAUUCAAAGUGGUUCAACUAAUUCAUCAACUGGAGAGGAGAAGGAAAGCGACAAUAAAAAACCAGA.. |
| Cdi-1-3 | AGAAUUAGGAGAGAAUAGUCAGAUUCAAAGUGGUUCAACUAAUUCAUCAACUGGAGAGGAGAAGGAAAGCGACAAUAAAAAG.. |
| Cpe-7-1 | UUGAAAGUAAUACUGUUGCUAAAGGCAUAAGUAAUAAUUUAAGAAUUGACUAUAAGAUUUUGAAUAAAGAUAAGCUUAAAGA.. |
| Cpe-1-1 | UUGAGAGUAAUACUGUGGCUAAAFACAUAAGCAAUAAUUUAAGAAUUGACUAUAAGAUUUUGAAUAAAGAUAAGCUUAAAGA.. |
| Cpe-2-1 | UUGAAAGUAAUACUGUUGCUAAAGACAUAAGCAAUAAUUUAAGAAUUGAUUAUAAGAUUUUGAAUAAAGAUAAGCUUAAAGA.. |
| Cpe-8-1 | UUGAAAGUAAUACUGUUGCUAAAGGCAUAAGCAAUAAUUUAAGAAUUGACUAUAAGAUUUUGAAUAAAGAUAAGCUUAAAGA.. |
| Cpe-4-1 | UUGAGAGUAAUACUGUGGCUAAAGACAUAAGCAAUAAUUUAAGAAUUGACUAUAAGAUUUUGAAUAAAGAUAAGCUUAAAGA.. |
| Cdi-1-4 | GUAAUUGCAAUUAUAGGUAUAUUAGCAGUAGUGGCAGUUCCAGCUUUAUUUAGUAAUAUAAACAAGGCUAAGGUAGCAAGUG.. |
| Cdi-2-3 | GUAAUUGCAAUUAUAGGUAUAUUAGCAGUAGUGGCAGUUCCAGCUUUAUUUAGUAAUAUAAACAAGGCUAAGGUAGCAAGUG.. |
| Cac-1-1 | AAACAUUGAUACACAUGAUUCGGAUAUAAAAAGUAUAAGAGAAUUUAAUGAGAGUUCAGAUAUAGUAAGAGAAGAUUCAGUA.. |
| Cno-1-1 | GAUGUUAUAAAAAGCAUAAAAAUAACUAAUGAAAAUGGACAAAAUAAAGAAACUUACAUUCCAGGUGAUAGAAUUAGGGUUG.. |
| Cte-1-1 | GGUUAUAGACCAGGUGAUAGAAUAAAAAUUGAUGUAGAUUGGUCUAUUAAAUCAGAAACAAAAGCUGGAGAUACCUUUAGUU.. |
| Cbe-1-5 | 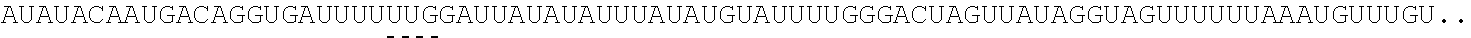 |
| Cbe-1-6 | UACAAUAUAGACGGGUACGAUAAAUAUGGAUAUGAUAAAGAAGGAUAUAAUAGUGGAGGAUAUGACAGGCAAGGAUAUAACA.. |
| Cac-1-2 | GUGGCUAAAUUGAGAAUUAUGAAGAUAAAUGUAGGAAAAUCAAUUUGAUUUUCCUACAUUUGUUUUUUGAAAAGGUAAAUAUA. |
| Env-8 | GCAAGACCUCGCACGCCCGCGGAGCGGCCUGCUCCGCAACCUGCCGCGCACGAUCCUGUUCGCGCUCGGCCUCAUCGGAUUAC. |
| Env-9 | AGCCCGAGUUGACGACGGAUGACAUCGUCUUCUCCGCCGCAACAAGCAGCACAACGGAGAUCAAGCAGCUCAAGGACAGCAC.. |
| Tca-1-1 | CUUGAAUUCCAGCUGGGAGUUCCUCAUAUCGACUUGGGAAGCGUGCCGCCCGAUCCGGAGGCAGCGGCGACUAUUCCGGCUU.. |
| | .................................................................................... |
| | .................................................................................... |
| | .................................................................................... |
| | .................................................................................... |
| | NNNNNNNNNNNNNNNNNNNNNNNNNNNNNNNNNNNNNNNNNNNNNNNNNNNNNNNNNNNNNNNNNNNNNNNNNNNNNNNNNNN |

Legend:
<-----> = basepaired region 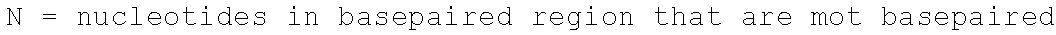
N = nucleotides in basepaired region that are not in Watson-Crick or G—U basepair (Outline text format)
NNNN = rho-independent transcription terminators
NNNN = perfectly fit consensu −35 and −10 box sequences

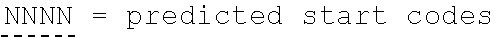

The RNAs reported herein are predicted to form two internal bulges using three base-paired regions (P1, P2 and P3), and an imperfect pseudoknot structure between nucleotides of the P3 loop (L3) and the junction linking stems P2 and P1 (J2-1) (FIG. 1A). The internal bulge between P2 and P3 conforms to a kink-turn motif (Klein et al. EMBO J. 20, 4214 (2001); Winkler et al. RNA 7, 1165 (2001)). Although, 24 of the 28 bacterial species that carry this riboswitch also carry c-di-GMP-I riboswitches, it was realized that representatives of the new RNA motif serve as aptamers for a distinct class of c-di-GMP riboswitches, termed c-di-GMP-II.

A representative RNA from C. difficile was subjected to in-line probing (Soukup and Breaker, RNA 5, 1308 (1999); E. E. Regulski, R. R. Breaker, Methods Mol. Biol. 419, 53 (2008)). This method exploits the instability of RNA phosphodiester linkages in unstructured portions of an RNA polymer to yield information on ligand-mediated structural changes and ligand binding affinities. An 84-nucleotide 5′ ³²P-labeled RNA (termed 84 Cd), corresponding to the conserved motif located upstream of a putative virulence gene (CD3246) in C. difficile strain 630 (FIG. 1B), was incubated in the absence or presence of 10 μM c-di-GMP under in-line probing conditions. Spontaneous RNA cleavage products were separated by denaturing polyacrylamide gel electrophoresis (PAGE), and band locations and intensities (FIG. 1C) were used to map regions of structural change. The observed pattern of cleavage products produced in the absence of c-di-GMP is consistent with the structural model generated by comparative sequence analysis. Furthermore, 11 internucleotide linkages in four spans within the L3 and J2-1 regions exhibit reduced strand scission when c-di-GMP is present (FIGS. 1B and 1C), which indicates that the RNA structure is stabilized by binding the second messenger.

In-line probing reactions using a range of c-di-GMP concentrations resulted in a binding curve with an apparent dissociation constant (K_{D}) of ˜200 μM (FIG. 1D). The concentrations of c-di-GMP range from a minimum of 260 fM to a maximum of 1.2 μM, with increments of 3-fold increases in concentration. This apparent K_{D} approaches the limit of sensitivity of the assay (Regulski and Breaker, Methods Mol. Biol. 419, 53 (2008)), and therefore the true affinity may be even better. Similarly, the affinities were determined for various analogs of c-di-GMP, revealing that the RNA discriminates against the linear form of the second messenger and other analogs by more than three orders of magnitude (FIG. 1E). The molecular recognition characteristics displayed by this RNA are similar to those observed for c-di-GMP-I aptamers (Sudarsan et al. Science 321, 411 (2008); K. D. Smith, et al. Nat. Struct. Mol. Biol. 16, 1218 (2009)), and therefore it is concluded that the RNA represents a new class of aptamers for this second messenger termed c-di-GMP-II.

i. c-di-GMP-II Aptamers are Components of Riboswitches

The 84 Cd aptamer from the CD3246 locus is not associated with any commonly-observed expression platform. Therefore, gene control function was confirmed for this aptamer class by using a representative RNA located upstream of the gene at the BH0361 locus from Bacillus halodurans (FIG. 2A). The BH0361 RNA is predicted to regulate gene expression by controlling the formation of an intrinsic transcription terminator stem (Soukup and Breaker, RNA 5, 1308 (1999); Regulski and Breaker, Methods Mol. Biol. 419, 53 (2008)). The ligand-bound state of this aptamer was predicted to preclude formation of an intrinsic transcription terminator (strong stem followed by a run of U residues) (Yarnell and Roberts, Science 284, 611 (1999); Gusarov and Nudler, Mol. Cell. 3, 495 (1999)), and therefore c-di-GMP binding was expected to activate gene expression.

A transcriptional fusion was created of the predicted riboswitch element or its corresponding M1, M2 and M3 mutants to a β-galactosidase reporter gene and transformed the construct into a surrogate organism, Bacillus subtilis. High levels of reporter gene expression were observed with the wild-type (WT) and compensatory (M3) constructs, but reduced expression resulted when two conserved bases in J1/2 were exchanged (M1), or when the P3 stem was disrupted (M2) (FIG. 2B). These constructs represent constructs with heterologous aptamer domains and expression platform domains and with heterologous riboswitch and coding region. Reporter gene fusions with WT or mutant motifs transformed into Bacillus subtilis confirmed the BH0361 locus RNA is a genetic ‘ON’ switch. These results are consistent with the proposed function of the B. halodurans BH0361 locus RNA as a genetic ‘ON’ switch.

Further evidence of aptamer-controlled transcription termination was observed by conducting single-round transcription assays in vitro using another C. difficile c-di-GMP-II construct from the ompR mRNA (FIG. 2C). As predicted from the expression platform architecture, the WT ompR construct also performs as an ‘ON’ switch and exhibits robust production of full-length RNA transcript only when c-di-GMP is added to the reaction (FIG. 2D). Mutations that disrupt P3 stem formation (M2) substantially reduce termination control, whereas additional mutations that restore P3 formation (M3) also restore function to near WT level.

ii. A Tandem Aptamer-Ribozyme Arrangement

The c-di-GMP-II aptamer from the CD3246 locus is very unusual because it lacks a typical riboswitch expression platform and it is located more than 600 nucleotides upstream of the putative AUG start codon for its associated open reading frame (ORF). The nucleotide sequence and structure of this unusually large gap correspond to a group I self-splicing ribozyme (Kruger et al. Cell 31, 147 (1982); Nielsen and Johansen, RNA Biol. 6, 375 (2009)) (FIG. 3A; FIG. 6). The predicted ribozyme has all the hallmarks of a typical group I ribozyme, including stems P1 through P10, and a U-G wobble base pair that defines a typical 5′ splice site (5′ SS).

The first step of group I ribozyme function normally is triggered solely by guanosine or one of its phosphorylated derivatives such as GTP. The 3′ oxygen atom of GTP functions as the nucleophile to initiate a phosphoester transfer reaction at the 5′ splice site (5′ SS). The second step involves a nucleophilic attack of the newly liberated 3′ oxygen atom of the 5′ exon to initiate another phosphoester transfer reaction at the 3′ splice site (3′ SS) to yield spliced 5′ and 3′ exons (5′E-3′E) and a linear intron or intervening sequence (IVS). The IVS can subsequently undergo one or more transesterification and hydrolysis reactions to create various circular and linear intron fragments (Yarnell and Roberts, Science 284, 611 (1999)).

Some bacteria exploit tandem riboswitch architectures to create sophisticated gene control systems that yield more digital gene control (Mandal et al. Science 306, 275 (2004); Welz and Breaker, RNA 13, 573 (2007)), two-input logic gates (Sudarsan et al. Science 13, 300 (2006)), or dual-mechanism switches (Poiata et al. RNA 15, 2046 (2009)). Furthermore, RNA engineering studies have demonstrated that the fusion of aptamer and ribozyme domains can produce allosteric constructs wherein ligand binding to the aptamer results in regulation of ribozyme activity (Breaker, Curr. Opin. Biotechnol. 13, 31 (2002); Silverman, RNA 9, 377 (2003)). Taking note of the existence of these more complex natural and engineered RNA switches, and of the fact that eukaryotic riboswitches that sense thiamin pyrophosphate (TPP) control nuclear splicing (Cheah et al. Nature 447, 497 (2007); Wachter et al. Plant Cell 19, 3437 (2007); Bocobza et al. Genes Dev. 21, 2874 (2007); Croft et al. Proc. Natl. Acad. Sci. USA 104, 20770 (2007)), it was realized and discovered that C. difficile 630 carries the first example of a natural allosteric ribozyme wherein c-di-GMP binding controls self-splicing. Only 10 nucleotides separate the 3′ terminus of the 84 Cd aptamer domain and the typical group I ribozyme 5′ splice site (5′SS) predicted for this RNA. Thus the aptamer is well positioned to regulate ribozyme access to one of its splice site junctions.

iii. An Allosteric Self-Splicing Ribozyme

Normally, group I ribozyme splicing is triggered exclusively by guanosine or one of its phosphorylated derivatives. The 3′ oxygen atom of the nucleoside functions as the attacking nucleophile to initiate a phosphoester transfer reaction at the 5′ splice site (5′ SS), which constitutes the first step of splicing. The second step involves a nucleophilic attack of the newly liberated 3′ oxygen atom of the 5′ exon to initiate another phosphoester transfer reaction at the 3′ splice site (3′ SS), which yields spliced 5′ and 3′ exons (5′E-3′E) and a linear intron or intervening sequence (IVS). The IVS can subsequently undergo one or more transesterification and hydrolysis reactions to create various circular and linear fragments of the intron (Kruger et al. Cell 31, 147 (1982)).

Allosteric control of the tandem aptamer-ribozyme arrangement was established in vitro by using 864-nucleotide internally ³²P-labeled RNAs (termed 864 Cd Tandem; FIG. 3A, FIG. 6) that carry both the aptamer and ribozyme domains, as well as additional nucleotides that form part of the ORF. Incubation of the wild-type 864 Cd Tandem RNA with GTP triggers splicing to yield several products that are characteristic of group I ribozyme activity (FIG. 3B). Importantly, the yield of spliced exons increases substantially when both GTP and c-di-GMP are present. The dose-response curve for c-di-GMP induction of splicing fits a 1-to-1 binding curve with half-maximal production of spliced exons occurring at a second messenger concentration of 30 nM (FIG. 7).

In addition, the yield of another RNA product (3′E*), subsequently determined to be a fragment of the 3′ exon caused by GTP attack far from the normal 5′ SS, is reduced in the presence of c-di-GMP (FIG. 3B). Aptamer mutants M1 and M2 that disrupt c-di-GMP binding no longer modulate product yields when the second messenger is included in the splicing reaction. In contrast, mutant M3 carries nucleotide changes to M2 that restore aptamer P3 stem formation and also recovers c-di-GMP-mediated splicing control (FIG. 3B). These results demonstrate that c-di-GMP addition and proper function of the adjoining aptamer trigger increased production of spliced exons by the ribozyme.

A comparison of ribozyme reaction products detected by using differently-radiolabeled RNAs and GTP was performed. The were products generated by using either (1) internally ³²P-labeled 864 Cd Tandem RNA and unlabeled GTP, or (2) unlabeled 864 Cd Tandem RNA and [α-³²P]GTP. Products were also generated by using either (1) internally ³²P-labeled 864 Cd Tandem RNA and unlabeled GTP, or (2) 5′ ³²P-labeled 864 Cd Tandem RNA and unlabeled GTP. Additional annotations are as described in the description of FIG. 3B. Kinetic assays conducted under similar conditions (see FIG. 4) reveal rate constants for unlabeled 864 Cd Tandem RNA and [α-³²P]GTP of 2.3×10⁻² min⁻¹ in the presence of both GTP and c-di-GMP versus 1.8×10⁻³ min⁻¹ when only GTP is present. There is only a small reduction in the initial rate constant for GTP attack at the alternative GTP₂ site (2.7×10⁻² min⁻¹ versus 2.3×10⁻² min⁻¹). Internally-labeled 864 Cd Tandem RNAs incubated with GTP exhibited kobs values of 1.1×10⁻³ min⁻¹ and 1.3×10⁻² min−1 in the absense and presence of c-di-GMP, respectively. Again, there is only a modest reduction in the initial kobs for production of the alternative GTP attack site product in the absence (2.4×10⁻² min⁻¹) versus the presence (1.8×10⁻² min⁻¹) of the second messenger. See also Table 5.

iv. Mechanism of Allosteric Splicing Control

Two alternative base pairing structures were noticed that could explain how c-di-GMP binding controls splicing. The first alternative stem, called anti-5′ SS (FIG. 3A, enclosed in dased line oval), includes the left shoulder of the aptamer P1 (nucleotides 8 through 17), which are complementary to nucleotides (90 through 99) that link the aptamer and ribozyme domains. The second alternative stem, called alternative ribozyme P1 (FIG. 3A, enclosed in alternating dash dot oval), includes the right shoulder of ribozyme P1 (nucleotides 186 through 189), which are complementary to nucleotides (667 through 670) near the 3′ end of the ribozyme.

Formation of the anti-5′ SS stem disrupts two base pairs of the ribozyme P1 stem. Since c-di-GMP is expected to stabilize the proposed aptamer structure, the absence of c-di-GMP will weaken the aptamer P1 stem, and favor formation of the anti-5′SS stem. Therefore, low concentrations of c-di-GMP can inhibit formation of the stem carrying the 5′ SS and prevent GTP attack at the 5′ SS. This was examined by conducting in-line probing on a 132-nucleotide construct encompassing the aptamer through nucleotide 130 of the tandem RNA. This construct isolates nucleotides predicted to be involved in forming the anti-5′ SS stem, and P1 stems of the aptamer and ribozyme. As predicted, c-di-GMP addition yields a spontaneous cleavage pattern consistent with aptamer P1 stem formation, while the absence of the second messenger permits the RNA to form the anti-5′ SS alternative pairing (FIG. 8A). The changes in the pattern of in-line probing data upon addition of c-di-GMP are consistent with formation of the alternative base-paired structure in the absence of ligand. Concentrations of c-di-GMP range from a minimum of 260 fM to a maximum of 1.2 μM, with increments of 9-fold increases in concentration.

Mutants M1 through M3 were prepared to examine the role of c-di-GMP binding on ribozyme splicing. Mutants M4 through M11 were prepared to assess other structures proposed to be important for allosteric ribozyme function, and mutant 12 was prepared to examine this group I ribozyme's usage of the typical guanosine binding pocket. The effects on self-splicing regulation of mutant M4, which retains aptamer P1 formation but disrupts the first alternative pairing, were also examined. M4 retains c-di-GMP binding activity (FIG. 8B), but splicing is no longer responsive to the second messenger, indicating that the ability to form the anti-5′ SS stem is necessary for allosteric control of ribozyme activity. Moreover, although M4 is no longer responsive to c-di-GMP, the yield of spliced exons is higher in the absence of the second messenger comparted to WT, which is expected if the aptamer P1 stem is not effectively competing with anti-5′ SS stem formation.

Construct M5, which carries mutations in the linker between the aptamer and the ribozyme, was expected to have similar activity as M4. This mutant loses splicing control as expected, but the RNA is defective in ligand binding and does not produce fully spliced products. Although the latter results are unexpected, in-line probing reveals that this mutant RNA is misfolded (FIG. 8C), which accounts for its lack of function. In contrast, placing M4 and M5 mutations in the same construct (M6) restores the potential for anti-5′ SS stem formation (FIG. 8D), and likewise restores allosteric activity of the construct. Collectively, the characteristics of these mutants strongly support the allosteric control mechanism involving mutually exclusive formation of aptamer P1 and anti-5′ SS stems. Similar experiments using additional mutants yield results that also support the formation of the alternative ribozyme P1 stem.

v. Abnormal Splice Site Junctions

Several lines of evidence reveal that this group I ribozyme uses splice and GTP attack sites that are distinct from most other group I ribozymes previously examined. This distinction is independent of allosteric regulation, but clarifies the origin of several reaction products. Sequence analysis of the 5′E-3′E splice product purified from in vitro assays revealed the exon junction sequence AGGAgGAGA, wherein nucleotides left of the lowercase letter correspond to 5′ exon nucleotides 97 through 100, while those on the right correspond to nucleotides 668 through 671. The lowercase nucleotide is derived from position 101, and not from 667.

More importantly, the splice junction lacks a U residue that is normally derived from the last nucleotide of the 5′ exon. This observation is striking because the ribozyme P1 stem carries a U-G wobble pair that group I ribozymes typically use to recognize 5′ splice sites (Strobel et al. Nat. Struct. Biol. 5, 60 (1998)). If GTP attack occurred as normal at the RNA linkage immediately following U102 in this wobble pair, then the junction between the spliced exons should carry this nucleotide at the terminus of the 5′ exon. However, the sequence of the splice junction indicates that the first step of splicing ignores this U-G wobble, and directs GTP attack at the preceding internucleotide linkage.

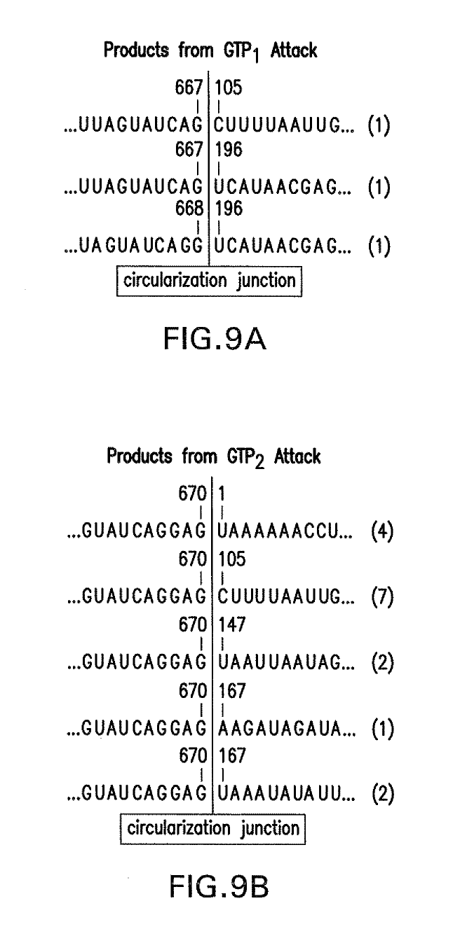

Unusual GTP attack and splice site use was also identified by sequencing the products of ribozyme circularization (FIG. 9), which the intervening sequence self-catalyzes after its excision from the precursor RNA. Of 19 clones examined, two exhibit circularization with nucleotide 667 and one with nucleotide 668, which are liberated for subsequent circularization by exon ligation at or near the established 3′ SS. The remaining clones involve circularization using nucleotide 670, which corresponds to the nucleotide liberated by GTP attack at an alternative site.

To assess whether the unusual 5′ splice site is biologically relevant, total RNA from cultured C. difficile 630 cells was isolated and subjected to RT-PCR using primers specific to the aptamer and the coding region of the tandem riboswitch-ribozyme system. The dominant PCR product isolated corresponds in size to that expected for a spliced 5′ UTR. Sequence analysis of this major product confirmed that this DNA represents spliced exons with a splice junction matching that found in the biochemical assays, thus indicating that this group I ribozyme naturally employs an abnormal 5′ SS. Furthermore, the extent of splicing increases with the age of the culture. This latter finding is consistent with allosteric activation of splicing by c-di-GMP, whose concentrations should increase with increasing cell density.

vi. Control of GTP Attack by c-di-GMP

To provide independent confirmation that the 5′ SS is atypical, in vitro splicing reactions were conducted using unlabeled 864 Cd Tandem RNA and ³²P-labeled GTP. Two radiolabeled products (short and long) were generated when [α-³²P]GTP was added to initiate the splicing reaction (FIG. 4A, FIG. 7). Interestingly, the inclusion of both [α-³²P]GTP and c-di-GMP results in substantially greater amounts of the long product, indicating that c-di-GMP binding regulates the first step of splicing by controlling the efficiency of GTP attack at one of two sites.

Precise GTP attack locations were mapped by purifying the radiolabeled reaction products and subjecting them to partial digestion with alkali and with RNase T1. The data reveals that the long RNA product results from GTP attack at the phosphorus center after nucleotide G101. Partial digests of long RNA products that carry a 5′-terminal ³²P-labeled GTP residue due to attack by [α-³²P]GTP were examined. The product band corresponding to cleavage after U102 is identified, which represents a dinucleotide wherein the source of the other nucleotide is [α-³²P]GTP added during the first step of splicing. The lowest band should correspond to [α-³²P]GTP>p, where >p represents 2′,3′-cyclic phosphate generated either by RNase T1 or alkali. Other annotations are as described in the description of FIG. 1C. This GTP attack site (GTP₁; FIG. 3A) corresponds to that required to produce the unusual splice site junction observed by sequencing the spliced products (5′E-3′E). Moreover, the short product is generated by GTP attack at the phosphorus center after nucleotide G670 (GTP₂; FIG. 3A), which is nearly 570 nucleotides downstream from the expected attack site. This data is consistent with the mechanism for c-di-GMP-mediated splicing control, wherein c-di-GMP controls ribozyme P1 formation and increases the probability of GTP attack at the 5′ SS. In the absence of c-di-GMP, GTP attack after nucleotide 670 within the alternative ribozyme P1 stem yields the product 3′E* (FIG. 3B).

vii. Mutations Support a Mechanism for Allosteric Ribozyme Function

Additional mutations were made to the 864 Cd Tandem RNA to more fully assess the mechanism of allosteric control (FIG. 3A). RNA splicing is eliminated by a C186G mutation (mutant M7), which is predicted to disrupt the base pair interactions immediately upstream of both the GTP₁ and GTP₂ attack sites in ribozyme P1 and alternative ribozyme P1 stems (FIG. 3A). The M8 construct, which carries the compensatory G101C mutation to M7, only restores normal GTP attack and splicing. Mutant M9 carries a G670C mutation that disrupts pairing of the alternative ribozyme P1 stem. As expected, M9 exhibits c-di-GMP-dependent allosteric regulation of splicing without the alternative GTP attack reaction occurring at GTP₂.

The double mutant M10, which combines the mutations from M7 and M9, yields only the GTP₂ attack product. Moreover, the triple mutation present in M11 should restore the potential for ribozyme P1 and alternative P1 stem formation, and indeed this construct mimics WT function. Mutant M12 carries a single C511U mutation that disrupts binding of the nucleophile GTP, rendering the ribozyme inactive. This latter observation confirms that group I ribozyme activity is required for any RNA processing to occur. Moreover, all these results are consistent with the involvement of the ribozyme P1 and alternative ribozyme P1 stems in directing ribozyme-catalyzed GTP₁ and GTP₂ attack, respectively.

viii. Rate Constants for c-di-GMP-Controlled Splicing

If competition exists between formation of the various (aptamer, ribozyme, and alternative ribozyme) P1 stems, then the rate constants for GTP attack and for the production of fully spliced exons will be altered by c-di-GMP binding. Rate constants for GTP attack were estimated by conducting splicing reactions with unlabeled 864 Cd Tandem RNA and [α-³²P]GTP (FIG. 4A). The yields of labeled RNA products due to attack at sites GTP₁ and GTP₂ (FIG. 3A) were quantitated, plotted (FIG. 4B), and used to derive rate constants. A near 13-fold increase in initial rate constant (first 15 minutes) was observed for GTP attack at the 5′ SS when both GTP and c-di-GMP are present (2.3×10⁻² min⁻¹) versus when only GTP is present (1.8×10⁻³ min⁻¹). In contrast, there is only a modest reduction in the initial rate constant in GTP attack at the alternative site (2.7×10⁻² min⁻¹ versus 2.3×10⁻² min⁻¹).

This same pattern of ribozyme modulation is observed when internally labeled RNAs are incubated under similar conditions (FIG. 4C). Plots of the yields of spliced exons (5′E-3′E) and of the product of alternative GTP attack (3′E*) (FIG. 4D) again reflect a 12-fold increase in the initial rate constant for spliced product formation in the absence (1.1×10⁻³ min⁻¹) and presence (1.3×10⁻² min⁻¹) of c-di-GMP. Likewise, there is only a modest reduction in the initial rate constant for production of the alternative GTP attack site product in the absence (2.4×10⁻² min⁻¹) versus the presence (1.8×10⁻² min⁻¹) of the second messenger.

Similar results are obtained by evaluating the yields of spliced exon and alternative attack products after incubating for 120 minutes (FIG. 4C, 4E). In the absence of c-di-GMP, the ribozyme produces more than 20 fold more alternative attack product than spliced exons (FIG. 4E, left). In contrast, near equal amounts of these two products are produced when c-di-GMP is present. This change is largely due to an ˜8-fold increase in the production of spliced exons, as the ratio of alternative attack product in the absence versus the presence of c-di-GMP drops only modestly (FIG. 4E, right). These assays reveal that c-di-GMP enhances the production of fully spliced exons largely by favoring formation of the ribozyme P1, but can gain a small increase in yield of spliced products by disfavoring formation of the alternative ribozyme P1 (FIG. 4E). Thus, c-di-GMP enhances the production of fully spliced exons largely by favoring formation of the ribozyme P1.

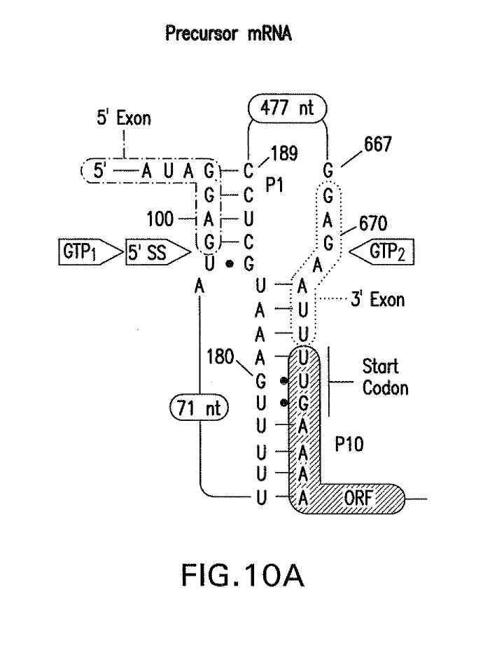

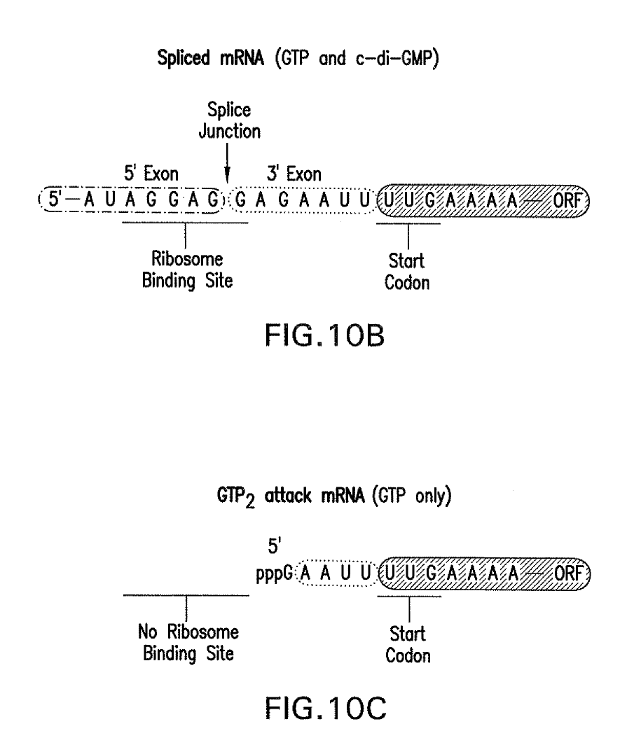

The sequences and structures of the precursor mRNA and processed RNAs suggest a mechanism for translation control of the CD3246 ORF (FIG. 10). Although the UUG start codon predicted in annotated C. difficile genomes is atypical, this translation start site and initial polypeptide sequence is consistent with similar genes in related organisms. In the precursor RNA, the start codon resides in the right shoulder of the P10 stem (FIG. 10A). This arrangement should restrict ribosome access and preclude translation, which is a common mechanism for translation control by riboswitches (Barrick et al., Genome Biol. 8:R239, 2007). With sufficient c-di-GMP and GTP, ribozyme action yields a processed mRNA wherein the spliced junction resides in a perfect ribosome binding site (AGGAGG) located an optimal distance upstream of the start codon (FIG. 10B). Thus, allosteric activation of ribozyme self-splicing by c-di-GMP should favor translation. In contrast, ribozyme action in the absence of c-di-GMP favors GTP attack only four nucleotides upstream of the start codon. This product is trimmed of nucleotides that otherwise could serve as a ribosome binding site (FIG. 10C), which should disfavor translation.

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a riboswitch” includes a plurality of such riboswitches, reference to “the riboswitch” is a reference to one or more riboswitches and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps.

Those skilled in the art will recognize, or be able to ascertain using no more than routine experimentation, many equivalents to the specific embodiments of the method and compositions described herein. Such equivalents are intended to be encompassed by the following claims.

1. A method of altering gene expression, the method comprising
bringing into contact a compound and a cell, wherein the cell comprises a gene encoding an RNA comprising a cyclic di-GMP-responsive riboswitch, wherein the riboswitch comprises a cyclic di-GMP-II motif.

2. The method of any of claim 1, wherein the cell has been identified as being in need of altered gene expression.

3. The method of claim 1, wherein the cell is a bacterial cell.

4. The method of claim 1, wherein the cell is a Clostridium cell.

5. The method claim 3, wherein the compound kills or inhibits the growth of the bacterial cell.

6. The method of claim 1, wherein the compound and the cell are brought into contact by administering the compound to a subject.

7. The method of claim 6, wherein the cell is a bacterial cell in the subject, wherein the compound kills or inhibits the growth of the bacterial cell.

8. The method of claim 7, wherein the subject has a bacterial infection.

9. The method of claim 6, wherein the compound is administered in combination with another antimicrobial compound.

10. The method of claim 1, wherein the compound inhibits bacterial growth in a biofilm.

11. A regulatable gene expression construct comprising
a nucleic acid molecule encoding an RNA comprising a riboswitch operably linked to a coding region, wherein the riboswitch regulates expression of the RNA, wherein the riboswitch and coding region are heterologous, wherein the riboswitch is a cyclic di-GMP-responsive riboswitch, wherein the riboswitch comprises a cyclic di-GMP-II motif.

12. The construct of claim 11, wherein the riboswitch comprises an aptamer domain and an expression platform domain, wherein the aptamer domain and the expression platform domain are heterologous, wherein the aptamer is comprised of the cyclic di-GMP-II motif.

12. (canceled)

13. The construct of claim 34, wherein at least two of the aptamer domains exhibit cooperative binding.

14. The construct of claim 1, wherein the riboswitch comprises the consensus structure of FIG. 1A or 5.

15. The construct of claim 11, wherein the riboswitch comprises an aptamer domain and an expression platform domain, wherein the aptamer domain is derived from a naturally-occurring cyclic di-GMP-responsive riboswitch.

16. The construct of claim 15, wherein the aptamer domain is the aptamer domain of a naturally-occurring cyclic di-GMP-responsive riboswitch.

17. The construct of claim 15, wherein the aptamer domain has the consensus structure of an aptamer domain of the naturally-occurring riboswitch.

18. The construct of claim 15, wherein the aptamer domain consists of only base pair conservative changes of the naturally-occurring riboswitch.

19. The construct of claim 1, wherein the aptamer domain comprises a P1 stem, wherein the P1 stem comprises an aptamer strand and a control strand, wherein the expression platform domain comprises a regulated strand, wherein the regulated strand, the control strand, or both have been designed to form a stem structure.

20. The construct of claim 1, wherein the aptamer domain comprises a control stem, wherein the control stem comprises an aptamer strand and a control strand, wherein the expression platform domain comprises a regulated strand, wherein the regulated strand, the control strand, or both have been designed to form a stem structure.

21. A riboswitch, wherein the riboswitch is a non-natural derivative of a naturally-occurring a cyclic di-GMP-responsive riboswitch.

22. The riboswitch of claim 21, wherein the riboswitch comprises an aptamer domain and an expression platform domain, wherein the aptamer domain and the expression platform domain are heterologous, wherein the aptamer is comprised of the cyclic di-GMP-II motif.

23. The riboswitch of claim 21, wherein the riboswitch is activated by a trigger molecule, wherein the riboswitch produces a signal when activated by the trigger molecule.

24. A riboswitch ribozyme comprising a riboswitch aptamer domain operably linked to a self-splicing ribozyme, wherein the aptamer is comprised of the cyclic di-GMP-II motif.

25. The riboswitch ribozyme of claim 24, wherein the aptamer domain comprises a control stem, wherein the control stem comprises an aptamer strand and a control strand, wherein the ribozyme comprises a regulated strand, wherein the regulated strand, the control strand, or both have been designed to form a stem structure.

26. The riboswitch ribozyme of claim 24, wherein the aptamer domain and the ribozyme are heterologous.

27. The riboswitch ribozyme of claim 25, wherein the riboswitch ribozyme is operatively linked to a coding region, wherein the riboswitch ribozyme and the coding region are heterologous.

28. A method of detecting a compound of interest, the method comprising
bringing into contact a sample and a riboswitch, wherein the riboswitch is activated by the compound of interest, wherein the riboswitch produces a signal when activated by the compound of interest, wherein the riboswitch produces a signal when the sample contains the compound of interest, wherein the riboswitch is a cyclic di-GMP-responsive riboswitch, wherein the riboswitch comprises a cyclic di-GMP-II motif.

29. The method of claim 28, wherein the riboswitch changes conformation when activated by the compound of interest, wherein the change in conformation produces a signal via a conformation dependent label.

30. The method of claim 28, wherein the riboswitch changes conformation when activated by the compound of interest, wherein the change in conformation causes a change in expression of an RNA linked to the riboswitch, wherein the change in expression produces a signal.

31. The method of claim 30, wherein the signal is produced by a reporter protein expressed from the RNA linked to the riboswitch.

32. A method comprising
(a) testing a compound for altering gene expression of a gene encoding an RNA comprising a riboswitch, wherein the alteration is via the riboswitch, wherein the riboswitch is a cyclic di-GMP-responsive riboswitch, wherein the riboswitch comprises a cyclic di-GMP-II motif,
(b) altering gene expression by bringing into contact a cell and a compound that altered gene expression in step (a),
wherein the cell comprises a gene encoding an RNA comprising a riboswitch, wherein the compound inhibits expression of the gene by binding to the riboswitch.

33. A method of identifying riboswitches, the method comprising
assessing in-line spontaneous cleavage of an RNA molecule in the presence and absence of a compound, wherein the RNA molecule is encoded by a gene regulated by the compound,
wherein a change in the pattern of in-line spontaneous cleavage of the RNA molecule indicates a riboswitch, wherein the RNA comprises a cyclic di-GMP-responsive riboswitch or a derivative of a cyclic di-GMP-responsive riboswitch, wherein the riboswitch comprises a cyclic di-GMP-II motif, wherein the compound is cyclic di-GMP.

34. The construct of claim 11, wherein the riboswitch comprises two or more aptamer domains and an expression platform domain, wherein at least one of the aptamer domains and the expression platform domain are heterologous, wherein at least one of the aptamer domains is comprised of the cyclic di-GMP-II motif.


SEQUENCE LISTING (122 sequences)

SEQ ID NO: 1 - protein, 39 aa, Artificial Sequence
  synthetic oligonucleotide; primer
TAATACGACTCACTATAGGTATTTATAGAAACTGTGAAG

SEQ ID NO: 2 - DNA, 22 bp, Artificial Sequence
  synthetic oligonucleotide; primer
tatttatagcaggttgcactac

SEQ ID NO: 3 - DNA, 43 bp, Artificial Sequence
  synthetic oligonucleotide; primer
atacgactcactataggtaaaaaacctatttatagaaactgtg

SEQ ID NO: 4 - DNA, 24 bp, Artificial Sequence
  synthetic oligonucleotide; primer
ggtacctgaccgtctgatggtttc

SEQ ID NO: 5 - DNA, 75 bp, Artificial Sequence
  synthetic oligonucleotide; primer
taatacgactcactataggtaaaaaacctatttatagaaactgtagagtatatcttaaacctgggcacttaaaag

SEQ ID NO: 6 - DNA, 50 bp, Artificial Sequence
  synthetic oligonucleotide; primer
atatcttaaacctgggcacttaaaacttatatggagttagtagtgcaacc

SEQ ID NO: 7 - DNA, 50 bp, Artificial Sequence
  synthetic oligonucleotide; primer
ggttgcactactaactccatataagttttaagtgcccaggtttaagatat

SEQ ID NO: 8 - DNA, 58 bp, Artificial Sequence
  synthetic oligonucleotide; primer
aacctatttatagaaactgtgaagtataagttaaacctgggcacttaaaacttatatg

SEQ ID NO: 9 - DNA, 58 bp, Artificial Sequence
  synthetic oligonucleotide; primer
catataagttttaagtgcccaggtttaacttatacttcacagtttctataaataggtt

SEQ ID NO: 10 - DNA, 61 bp, Artificial Sequence
  synthetic oligonucleotide; primer
taatacgactcactataggtaaaaaacctatggatagaaactgtgaagtatatcttaaacc

SEQ ID NO: 11 - DNA, 58 bp, Artificial Sequence
  synthetic oligonucleotide; primer
atatggagttagtagtgcaacctgctatccatataaataggagtaacttttaattgtc

SEQ ID NO: 12 - DNA, 58 bp, Artificial Sequence
  synthetic oligonucleotide; primer
gacaattaaaagttactcctatttatatggatagcaggttgcactactaactccatat

SEQ ID NO: 13 - DNA, 59 bp, Artificial Sequence
  synthetic oligonucleotide; primer
tatatggagttagtagtgcaacctgctataaatatccataggagtaacttttaattgtc

SEQ ID NO: 14 - DNA, 59 bp, Artificial Sequence
  synthetic oligonucleotide; primer
gacaattaaaagttactcctatggatatttatagcaggttgcactactaactccatata

SEQ ID NO: 15 - DNA, 53 bp, Artificial Sequence
  synthetic oligonucleotide; primer
gagttagtagtgcaacctgctatccatatccataggagtaacttttaattgtc

SEQ ID NO: 16 - DNA, 53 bp, Artificial Sequence
  synthetic oligonucleotide; primer
gacaattaaaagttactcctatggatatggatagcaggttgcactactaactc

SEQ ID NO: 17 - DNA, 49 bp, Artificial Sequence
  synthetic oligonucleotide; primer
gatagatataaatatatttttgaaatggtccctgcattcataacgagtg

SEQ ID NO: 18 - DNA, 49 bp, Artificial Sequence
  synthetic oligonucleotide; primer
cactcgttatgaatgcagggaccatttcaaaaatatatttatatctatc

SEQ ID NO: 19 - DNA, 64 bp, Artificial Sequence
  synthetic oligonucleotide; primer
gtagtgcaacctgctataaatataaataggactaacttttaattgtcaaaaatatataaaataa

SEQ ID NO: 20 - DNA, 64 bp, Artificial Sequence
  synthetic oligonucleotide; primer
ttattttatatatttttgacaattaaaagttagtcctatttatatttatagcaggttgcactac

SEQ ID NO: 21 - DNA, 71 bp, Artificial Sequence
  synthetic oligonucleotide; primer
ttaagtgaatacaatatatgtttttagtatcaggacaattttgaaaacaaaaattaaaaaatcaagtataa

SEQ ID NO: 22 - DNA, 71 bp, Artificial Sequence
  synthetic oligonucleotide; primer
ttatacttgattttttaatttttgttttcaaaattgtcctgatactaaaaacatatattgtattcacttaa

SEQ ID NO: 23 - DNA, 54 bp, Artificial Sequence
  synthetic oligonucleotide; primer
ggagtacctaatttatggttaggtaatgatatagtttgtgcttaatagaaatat

SEQ ID NO: 24 - DNA, 54 bp, Artificial Sequence
  synthetic oligonucleotide; primer
atatttctattaagcacaaactatatcattacctaaccataaattaggtactcc

SEQ ID NO: 25 - DNA, 33 bp, Artificial Sequence
  synthetic oligonucleotide; primer
ttttatatatttttgacaattaaaagttactcc

SEQ ID NO: 26 - DNA, 88 bp, Artificial Sequence
  synthetic oligonucleotide; primer
ggagcataaaaaatcaatagggaagcaacgaagcatagcctttatatggacacttgggttatgtggagctactagtgtaaccggccct

SEQ ID NO: 27 - DNA, 88 bp, Artificial Sequence
  synthetic oligonucleotide; primer
agttttcataaaaaaaagacacctctcctattgttaaaggagggccggttacactagtagctccacataacccaagtgtccatataaa

SEQ ID NO: 28 - DNA, 96 bp, Artificial Sequence
  synthetic oligonucleotide; primer
tgagaattctacgacaaattgcaaaaataatgttgtccttttaaataagatctgataaaatgtgaactaaggaggataaaaaatcaatagggaagc

SEQ ID NO: 29 - DNA, 67 bp, Artificial Sequence
  synthetic oligonucleotide; primer
gatgaattctacgacaaattgcaaaaataatgttgtccttttaaataagatctgataaaatgtgaac

SEQ ID NO: 30 - DNA, 31 bp, Artificial Sequence
  synthetic oligonucleotide; primer
ctggatccagttttcataaaaaaaagacacc

SEQ ID NO: 31 - DNA, 21 bp, Artificial Sequence
  synthetic oligonucleotide; primer
gggaagcaacagagcatagcc

SEQ ID NO: 32 - DNA, 21 bp, Artificial Sequence
  synthetic oligonucleotide; primer
ggctatgctctgttgcttccc

SEQ ID NO: 33 - DNA, 40 bp, Artificial Sequence
  synthetic oligonucleotide; primer
agggaagcaacgaagcatatgctttatatggacacttggg

SEQ ID NO: 34 - DNA, 40 bp, Artificial Sequence
  synthetic oligonucleotide; primer
cccaagtgtccatataaagcatatgcttcgttgcttccct

SEQ ID NO: 35 - DNA, 46 bp, Artificial Sequence
  synthetic oligonucleotide; primer
tatgctttatatggacacttggcgtatgtggagctactagtgtaac

SEQ ID NO: 36 - DNA, 46 bp, Artificial Sequence
  synthetic oligonucleotide; primer
gttacactagtagctccacatacgccaagtgtccatataaagcata

SEQ ID NO: 37 - DNA, 62 bp, Artificial Sequence
  synthetic oligonucleotide; primer
ggtacctgaccgtctgatggtttctctacatctggtgtctgaccatttgattgttcttctcc

SEQ ID NO: 38 - DNA, 44 bp, Artificial Sequence
  synthetic oligonucleotide; primer
gaccctaattgaacacactaaagatgtattattgagtaagattc

SEQ ID NO: 39 - DNA, 43 bp, Artificial Sequence
  synthetic oligonucleotide; primer
ctaattttttctactttcgtaacctcaagtgcttattaaatgc

SEQ ID NO: 40 - DNA, 26 bp, Artificial Sequence
  synthetic oligonucleotide; primer
atcttatatctaagaatatggaaata

SEQ ID NO: 41 - DNA, 40 bp, Artificial Sequence
  synthetic oligonucleotide; primer
tactcttattttcaaattttgcaacatttttgttaatttt

SEQ ID NO: 42 - DNA, 37 bp, Artificial Sequence
  synthetic oligonucleotide; primer
acaaaaaaagactatgcaatataaaaattatattcat

SEQ ID NO: 43 - DNA, 43 bp, Artificial Sequence
  synthetic oligonucleotide; primer
ttttagaaactgagaagtataagttattattgggcatctggag

SEQ ID NO: 44 - DNA, 43 bp, Artificial Sequence
  synthetic oligonucleotide; primer
ctccagatgcccaataataacttatacttctcagtttctaaaa

SEQ ID NO: 45 - DNA, 64 bp, Artificial Sequence
  synthetic oligonucleotide; primer
ttttagaaactgagaagtataagttattattgggcatctggacttatatggagttagtggtgca

SEQ ID NO: 46 - DNA, 64 bp, Artificial Sequence
  synthetic oligonucleotide; primer
tgcaccactaactccatataagtccagatgcccaataataacttatacttctcagtttctaaaa

SEQ ID NO: 47 - DNA, 27 bp, Artificial Sequence
  synthetic oligonucleotide; primer
ccaatagtatcttatgctgacgaagtg

SEQ ID NO: 48 - DNA, 28 bp, Artificial Sequence
  synthetic oligonucleotide; primer
gtgaagtatatcttaaacctgggcactt

SEQ ID NO: 49 - RNA, 26 nt, Artificial Sequence
  Consensus sequences for c-di-GMP-II riboswitch
nnnnnrgarrcnnugannyrynnnyu

SEQ ID NO: 50 - RNA, 12 nt, Artificial Sequence
  consensus sequence for c-di-GMP-II riboswitch
aynuggrcacyu

SEQ ID NO: 51 - RNA, 26 nt, Artificial Sequence
  consensus sequence for c-di-GMP-II riboswitch
gagyyagurgugcaaccgrcynnnnn

SEQ ID NO: 52 - RNA, 84 nt, Clostridium difficile
gguauuuauagaaacugugaaguauaucuuaaaccugggcacuuaaaagauauauggaguuaguagugcaaccugcuauaaaua

SEQ ID NO: 53 - RNA, 121 nt, Bacillus halodurans
ggagcauaaaaaaucaauagggaagcaacgaagcauagccuuuauauggacacuggguuauguggagcuacuaguguaaccggcccuccuuuaacaauaggagaggugucuuuuuuuuaug

SEQ ID NO: 54 - RNA, 119 nt, Clostridium difficile
aauaaaaauauuuuagaaacugagaaguauaucuuauuauugggcaucuggagauauauggaguuaguggugcaaccggcuaugaauauaauuuuuauauugcauagucuuuuuuuuuu

SEQ ID NO: 55 - RNA, 105 nt, Clostridium difficile
gguaaaaaaccuauuuauagaaacugugaaguauaucuuaaaccugggcacuuaaaagauauauggaguuaguagugcaaccugcuauaaauauaaauaggagua

SEQ ID NO: 56 - RNA, 15 nt, Clostridium difficile
uuuuugaaaugcucc

SEQ ID NO: 57 - RNA, 15 nt, Clostridium difficile
ggagaauuuugaaaa

SEQ ID NO: 58 - DNA, 46 bp, Artificial Sequence
  consensus sequence for c-di-GMP-II aptamer
nnnnannggcaaayynnnygnaarnnnrrgacgcaaagcyyrggyc

SEQ ID NO: 59 - RNA, 14 nt, Artificial Sequence
  consensus sequence for c-di-GMP-II aptamer
grycynrgyugccr

SEQ ID NO: 60 - RNA, 105 nt, Clostridium difficile; identical to SEQ ID NO: 55

SEQ ID NO: 61 - RNA, 105 nt, Clostridium difficile
gguaaaaaaccuauggauagaaacugugaaguauaucuuaaaccugggcacuuaaaagauauauggaguuaguagugcaaccugcuauccauauaaauaggagua

SEQ ID NO: 62 - RNA, 105 nt, Clostridium difficile
gguaaaaaaccuauuuauagaaacugugaaguauaucuuaaaccugggcacuuaaaagauauauggaguuaguagugcaaccugcuauaaauauccauaggagua

SEQ ID NO: 63 - RNA, 105 nt, Clostridium difficile
gguaaaaaaccuauggauagaaacugugaaguauaucuuaaaccugggcacuuaaaagauauauggaguuaguagugcaaccugcuauccauauccauaggagua

SEQ ID NO: 64 - RNA, 20 nt, Artificial Sequence
  synthetic construct; junction of ribozyme self- circularization product
uuaguaucagcuuuuaauug

SEQ ID NO: 65 - RNA, 20 nt, Artificial Sequence
  synthetic construct; junction of ribozyme self- circularization product
uuaguaucagucauaacgag

SEQ ID NO: 66 - RNA, 20 nt, Artificial Sequence
  synthetic construct; junction of ribozyme self- circularization product
uaguaucaggucauaacgag

SEQ ID NO: 67 - RNA, 20 nt, Artificial Sequence
  synthetic construct; junction of ribozyme self- circularization product
guaucaggaguaaaaaaccu

SEQ ID NO: 68 - RNA, 20 nt, Artificial Sequence
  synthetic construct; junction of ribozyme self- circularization product
guaucaggagcuuuuaauug

SEQ ID NO: 69 - RNA, 20 nt, Artificial Sequence
  synthetic construct; junction of ribozyme self- circularization product
guaucaggaguaauuaauag

SEQ ID NO: 70 - RNA, 20 nt, Artificial Sequence
  synthetic construct; junction of ribozyme self- circularization product
guaucaggagaagauagaua

SEQ ID NO: 71 - RNA, 20 nt, Artificial Sequence
  synthetic construct; junction of ribozyme self- circularization product
guaucaggaguaaauauauu

SEQ ID NO: 72 - RNA, 15 nt, Artificial Sequence
  precursor mRNA
uuuuugaaaugcucc

SEQ ID NO: 73 - RNA, 15 nt, Artificial Sequence
  precursor mRNA
ggagaauuuugaaaa

SEQ ID NO: 74 - RNA, 21 nt, Artificial Sequence
  spliced mRNA with ribosome binding site
auaggaggagaauuuugaaaa

SEQ ID NO: 75 - RNA, 11 nt, Artificial Sequence
  mRNA with no ribosome binding site
aauuuugaaaa

SEQ ID NO: 76 - RNA, 474 nt, Artificial Sequence
  synthetic construct; env-1 c-di-GMP-II riboswitch aptamer
ucagaauagguuguuaguucgggcggcgaagauccggugacuggcauggucgcuccauccuuucgggauucagaauagguuguuaguucgggcggcgaagauccggugacuggcauggucgcuccauccuuucgggauacggucacugaggagccaguagcgaaaccggcccuaugggucgguuuguuuguuuaaaggaacccgaaggaggccguucuucccaacacgcgugacuccuuuuccacuuaucuuuuccauucgaagacgauuagaccaggaggaccuaugauuucaagacgagacuuucuaaaauugagcggcgcgggcauccuuucguuguauggcguuucgcgcggaaaggucacuuugcgugcccaggcccagaucccgggcggcacacucaacccccaugacgugcccaaauauaugacgccccugcucauuccgccggucaugcccaaggccggaacgauc

SEQ ID NO: 77 - RNA, 495 nt, Artificial Sequence
  synthetic construct; env-2 c-di-GMP-II riboswitch aptamer
aucugcccaggagcgcacguaggccggccgguucucgauuagcacgccaucacgguccaggaagauugccggguacauaagggcaauuauaaccuucaccacuguggacgcuuugauccggugucuugauuuggucgcuuaacgucuggaacuuuugacgagggcgccggggagcgaguagcgaaaccggccacugguacugcggccgguuuugucuguuuaaacagcccggaggcgccgcgggcguaaauucgaccgaaaaucgacuggaaccgcucgauccagacgggggaucguucuauaauuggagcagcggggugucccugcaagcucaaugagaauaugaucgaacagcugcaacuuuaccucagccgccaaucuuccagccugggccguuaugugcuggaacaaagcauauaugcgcuggcggguuggaucccgucaauuguuggcauugccgugcgcggccugcucuaccgucugauccugcacaug

SEQ ID NO: 78 - RNA, 475 nt, Artificial Sequence
  synthetic construct; env-3 c-di-GMP-II riboswitch aptamer
auugacgagugaagccuaguugccgcgaugggcuugacuucucgagcacggguagccuacaguaccgaaugagguaguacuuucguacgccuuugaaucgauuucggaugccuugauccgaccgcugaaugugggcacuuaggcggccggggagcagguagugcaaccgaccguuccgcaccggagugugucgguuguguguauuucgucucagaagagggcaccuucugaugguuagagagcugaucaagggcugguagcccuuccgaggagaauucggcaugcgagcgcacuucaugcgcguuggcgcgcugaugucaguccucggcacgcuggcugcccucgcccuggcggggggagccggcguucgaggcugguagggggccgugguagacuccggccgcgcuugacgcuuagcgauugccggugnguccaacguugcccgccuaguccucauuacaguagccgccgcagu

SEQ ID NO: 79 - RNA, 476 nt, Artificial Sequence
  synthetic construct; env-4 c-di-GMP-II riboswitch aptamer
gaucggucgacguucucaucgugauucgcacuccgccuucuccgaucuccccuccacccgaaacggucaagcacccaaucguuggacgccuccugcgaggcguccggaggcgaugacccgcugauccccauuugggcaucaagggacgcggcgagccagugaugcaaccggccggacggcuacgcacguguguacguggccccaggccucggaggaggaacgaugcaagaccucgcacgcccgcggagcggccugcuccgcaaccugccgcgcacgauccuguucgcgcucggccucaucggauuacucgucacgggauguuccacguccgccuugcccagcgagacccucgccgaccccgucaucgaguucacgauccccgcgacccucaccccaaccuucgaagacacgcaggugcaacgguuggacggcacgacgcucgagagugacgugagcgucuucguucgcgaucaccg

SEQ ID NO: 80 - RNA, 474 nt, Artificial Sequence
  synthetic construct; Ame-1-1 c-di-GMP-II riboswitch aptamer
guguagaaaagaggguuucguuguauaauguagaacuguguaauaguaauaaguguuggauauuguuaauaguuugaucaagugaagcauuaaagcgaaaaauguagagcaaaugaacugcagguauacauggacgccuuaaacugcagggauguaguggcguaaccgacuaacaauauucaauuagauuguuagucucuauuuuuuugaauauaaaucgauuucaugaacugaggagaauaaaaugaugcaaaaaaauaugcguuuaggggacauccugauugaaggagguuugauaacaaaagaucaacuuaaugagauacugaagcuucaaaaaauaaagggaaaaaaauuaggagagcuuaucguugaugaagggauuuuaaaagaaagucagauucuagagguuuuagaauuucaauugggcauaccacauauugauuuaaauaaauaucauauuaauccuaaggcagu

SEQ ID NO: 81 - RNA, 393 nt, Artificial Sequence
  synthetic construct; env-5 c-di-GMP-II riboswitch aptamer
aguugauuauccccaccccgaguuaaauagcucaaauucguugacagaagcauucacgaguuauacaauaagcuccuaagacuaaaauguggguuaaauaauuuggugggcguugaugugcccuuuguaucuggucgcuugagggguacggagccaauagcgaaaccgccgccgucauagaggggagcacuaugaucgcuagucgguugcgcaaaauaacaaaaaagauuaagaacucgguugaaaugggcuuggcggcaguacugcuacugagucaguuugcuuuuacauuuggcacguuaauuaacgcgacaucgguauacgccgcaaacgauauaacugcuuccaaccacgaaggccagcucuccccugcaggcacuugguccaauggca

SEQ ID NO: 82 - RNA, 474 nt, Artificial Sequence
  synthetic construct; Tet-1-1 c-di-GMP-II riboswitch aptamer
gcuaauuauucuuucaaauuauggacagggggauuuuucgacaaaaauuugguauacagguauuuaaaagaaggaauuacgcauaauuuguagaauauuuuauaaagaguagaugaagugaggcuauaugugggcacuuuaugccucacggauacaguggugcaaccggcuuuaugucggcuuuauuauuuuuggagguauauuuuauggccaagaaaaagcuaggggagcuuuuacuugagguagggcuuauaacagaagaacaauuaaagcacgcaauagaaguucaaaacaaaacaggagaaaaguuaggaaaaguucuuauuaaauugggguauguuacagaaagccaaauacuagaggcuuuagaauuucaauuagguauuccacauguagauuugcaaaaauacuauauugaucccgauguagcaaaauuaauuccugaagccguugcuaaaagacauacuauuauuc

SEQ ID NO: 83 - RNA, 478 nt, Artificial Sequence
  synthetic construct; Pth-1-1 c-di-GMP-II riboswitch aptamer
acauuuggcauauauugacacaaaauauaaaaaccgaauauaauuaacguaaaauauaaaauauuaaaauaaaagcauuaaauaaaucugauuuuagagauagugugaagcagugaucuugcagcuaauauggucacuuuuuacggcugcaagugagcuaauagugaaaccggcacacugaugcccgguuuuuuuguuuauuaauaauggcagaugaugguuaggcaguuauuaaauucuaaggggggugaaaauauuuauagauaacaaacuguaccgcauuacagcacugccaugaccggaugccuugccgaaaagacugucuugcggcaaaaaaaugcuugagcaaaaggcggugguauuuauguccauuuauaaccgaaagaaaagucuuaaggccuuauucagaauaaguuuacuugcuguuuuacuguugugccucauuccguuacacaaagcgucagccgcuuccggcgaa

SEQ ID NO: 84 - RNA, 475 nt, Artificial Sequence
  synthetic construct; Dra-1-1 c-di-GMP-II riboswitch aptamer
guugugagaaaucucauauuuggaagugcuuccugagccauuauaaagaaguccccgcgcaccccaugccuggccguaugaggagugugaacgacgaagugguuuugaagcagggaagcaucugacgcauucgggcgccuugucagauguggagcgaguggcgcgaccggcaaagcuguggcaacacacuugccgguuguuuguugucucucggcuggccccgcccucuuauguucugaacgcuuuucucaaggaguugcacaugcguacauucucuucucucuccgcuguggucgcagcacucgcacuggguacugcuggggcacagacccucgcuacuccccccacuacucccgcgggcacggagaucaucaacaaggcggaaaucaccuucacgcccgaauccaccccgaccaaccccaacccccccagcgaaaccauugagacgccgcccgucaucacgguggucaacccg

SEQ ID NO: 85 - RNA, 474 nt, Artificial Sequence
  synthetic construct; Dge-1-1 c-di-GMP-II riboswitch aptamer
caaaagucacuuugcuuccuuuuugugcugcuguugugacgagcugcuguugugaguuggcaguaaggguucuguuagaggugccuagauaccuucuucuuguuccggggcgaagaggcgugugguguauccgggcaccugccacacgcggagccaguggugcgaccgacgaaccgugaacccuuuggggaucgcguucgugggucgcuuuuugcugccuaccucucggccccgcuguuuucaacgcucgacccuuuuucugcucuccagcgauggugcuuccgccgcugugggaagcgcuucuauccccuuugaggaggagccaugaagcucaaaguuuuucugccaguacuccuggugucgggcgcggcccucgcccaagacaccucgccucuccagcuggucuacgaccaggugcugauccagaccgucacgcagaacggcaaagccaccgagaaacgcacgccgggcgug

SEQ ID NO: 86 - RNA, 475 nt, Artificial Sequence
  synthetic construct; Dra-1-2 c-di-GMP-II riboswitch aptamer
uggaaagaaucagggcgcauuaagacgccuuaagaguucugucagagaucccacuuuagacucgcuucacuucgcacagauucaccccaggaacuucugucgaagagacgcgaugaaucccgcccuguaauucgggcaccucggacgggaggagcaaguggugcgaccggcuuuucguuggugucuggccccggagcuguuccaggcgucuggcccuuauuucgcggccccugccgcuuucaggaguucucauguucaauaccagaaagucugcccucgucaucgccgcccugcucagccucggggcccccgcccuggcggggacgagcagcacccagaccgacuacgacaacgcuacccugagccugagcgccgacggcaccuaugcccaggccgucagcuucagcgugcccagcaccaccgucacgcucagcgccgagcagauucgccccggcaacagcuucacgcucuccau

SEQ ID NO: 87 - RNA, 418 nt, Artificial Sequence
  synthetic construct; env-6 c-di-GMP-II riboswitch aptamer
ngguuuunnnggauuguauagcccgauucuguggaggccugauccauagcgguacuggucgcagcucuuucuggauagaaagaacgcuauuggggagccaguugcgaaaccggccacguagccgguuuuugcgucuuaagugccugacgggggucagaaaagcuggucuaaccucuucgaagccaauucugcugucacauccuugccuucaacgcuccccucagaagucgcugcgcuucucuuuguuuacuucggaucugucagugaacgagaaaggagacgaaagcacaccaucucucaaccaucuucaagcauguucgaacccacaacaaguucuauucgagauauuggaugaauuugccuaaggagaaacagaugcuucacagaauaucgaucguauuucgagcccugcugaucg

SEQ ID NO: 88 - RNA, 473 nt, Artificial Sequence
  synthetic construct; Bsp-1-1 c-di-GMP-II riboswitch aptamer
aaaugaauaaaauauauauugacuuaauaugacaggaaucauauauuuuaauuggcuugcaauugucuguuucgacagaaaaugcaaggguguuuuaagaauauagaacacugugaugagcgguuuuuauuugcacuuuaaaccgcuuggagugacuagugcagccggccaaugaucuaugauggcugguuuuuauuuuggccggggccucugcuuccuggcugaugcuuaggaugaaagaggugcauuuaaugaagcuugauaaauacaaaacuuuauuguuucaaaagauuaagaaucaaaugacucuuugguuugaacaggaagcggaaacuccugugaacaacgaagauguuuaccguuuccuucacuccaucaaggggacagcaggaacucugcagcuugucggacuccaucagguggcaggcaaacugauggaccagguggaaaagagcagcgaaaaaaucugggga

SEQ ID NO: 89 - RNA, 475 nt, Artificial Sequence
  synthetic construct; Dsp-1-1 c-di-GMP-II riboswitch aptamer
aggauauagcagugguaauguauaguaaugauaauauaguaucaguuauauugacuuugcaucuuuugcaggccaauaaugguauuggaaucagcaaaauuaaggggaagcguugagccgcuacccauaugugguucacucggauagcggggagcuaauagugaaaccggcccuuuaggggucgguuuuguuuuuggucaaauuauagagaugcuuauaaggaggugcucugaauuaagugagauugccgguaguacauagauagcaauucaaaggagggagagcuuauuaugaaaagagcacuaucaauauugcuuucacuuucaauucugguaagcuugggauuguuuuccgcugugccuguaagcgcggcgauaguaaauguaucuaucagcagcggcacuucaacugagauuguaggcguauacaauaaggcuggagggggaucaguauacguugaccuaagcgguucacc

SEQ ID NO: 90 - RNA, 347 nt, Artificial Sequence
  synthetic construct; Hor-1-1 c-di-GMP-II riboswitch aptamer
gauuuuucuugacuaugggaaaaaaguaggauauaauaaaauugaaauuuguauaauuauccaauaguuuaagguauuuauuguaaaauuuaauaauauugaaugggaagcuaugaacucuucuuuguacguggucacuugaaggagagggagcuaguagugaaaccgccccgaccgggggcgguuuuugcuuuuuacauaccuuuaaaauauuucaggggggggauuuuuaaugaaaaaagguuuaauuuuuggacuggcuguaguguugacaauggugcuuucuuuaggcguuauggcagcagguaaugauucgggcacuguaaauguaacugcuucugugcaaa

SEQ ID NO: 91 - RNA, 474 nt, Artificial Sequence
  synthetic construct; Bha-1-1 c-di-GMP-II riboswitch aptamer
guuauuccuguuaaaaguaauaaccuuagacaaaauuguaucauuuuuaagaagucggaccaaauuaaaugccucguguaauaucggagcauaaaaaaucaauagggaagcaacgaagcauagccuuuauauggacacuuggguuauguggagcuacuaguguaaccggcccuccuuuaacaauaggagaggugucuuuuuuuuaugaaaacuuugaaacauaucagcuacaaauuacggauauuugcauugauugcauuacucauuucacaaacguugcuuacgagcuuaagucuuccauuucaagcaguagccaauuccgaaaagaguggacuaacgaauguggaaauaaccgaugcgaauggacaaacaauugaugcagaucaauuuccagaccguuucguuucgaucgauucgauuuuacaaaucauccuuucauggagcguugacgaaaguguagcacagggggcggag

SEQ ID NO: 92 - RNA, 477 nt, Artificial Sequence
  synthetic construct; Ckl-1-1 c-di-GMP-II riboswitch aptamer
guuauuagagugauuugagugaaaguuuuaauauuuggcaguuaaauuauuuuccaaaguuuauugaaacuggaguguuuuuaugcuaaaauuaaacaaauugaggggaacgaugagguacacacuuuaauuugggcauccuuguguguauuggaguuagugaugcaaccgacccuguauucauaaauauuuuauggauacaggguuuugcuuuuuaaaaauggagaauaacuauggauauuuauuaugguauuuuggcuuuuauuuuuggaacuaucauaggaagcuuuuuaaauguuuguauauauagaauaccuaaaggugagucuauuuuacauccaccuucucauuguaugaauuguaauaaaaaaaucaaggccuaugauaugauuccuguaauaaguuauaucauuuugagaggaagaugcagauauugcggcaaaaagauacauuuaagguauccugucuuagaauuua

SEQ ID NO: 93 - RNA, 474 nt, Artificial Sequence
  synthetic construct; Cbu-1-1 c-di-GMP-II riboswitch aptamer
uaaguuaaaaaugauauuauauauauggaaauagaaaaaaaugguauauaauuacauuauguuagguuauauauaaguaaaaauaaaauagauuaucauguaguuggaaacgaagaauaguucucucaaucugggcaccuugggaacuaggaguuaguggugcaacccgccagcaaauuaauuaguauuaauuuguugguuuuuuuuauaaaaaauaagaggaaaaaauagauuauuaugagauuuauguagauauaggaggauuuauaaacuuggauuauauauuaguaauaauauuaggauugguuauaggaaguuuuuuaaauguuuguauuuauagaauuccaagagaagaaucaauuucguauccaccaucucauuguggaaacugcggucauaaucuacagccuguagauuuaauuccuauaauaaguuauguguuuuuaaaaggaaaaugcagauauuguaaggaga

SEQ ID NO: 94 - RNA, 474 nt, Artificial Sequence
  synthetic construct; env-7 c-di-GMP-II riboswitch aptamer
ugcauauuuuaaugcaaauaauuauauuuuuuaucaaaauaugcuugaauauauggaguuauuagaauauuauauauauaaauucuaugaaaacauaauaaaucuggaaguaaugauaaauucucuuuauuugggcacuuugaggauuuugaacuaguagugcaacccgccaacaauuaaucaagauuguuggcguuuuugaugauagaaaauaugugaaaauuuaugaauaaggaaauauauacuagauuuuaauuauacauaaguauuuauuuauauauuaaugaaauuacugauauauauguucuaauuaagaaucuucauuaaaaucugcccuaaacccaaaaaggauauauggcucauaugaaguaauuuuauuggaacuuauauaaauacaaauaaacuuauuauaaaaauuaaauaaauuauuaaaagggguugauauauugauuauaaggcgguuaaguauaggcc

SEQ ID NO: 95 - RNA, 474 nt, Artificial Sequence
  synthetic construct; Cbe-1-1 c-di-GMP-II riboswitch aptamer
aggaaauuaacaaaaagauaguuagauuauugauuuuuuaauaucuauaugauaauaugacauauauauuaaaaaaaugacaauuauuaaugaaauauaguaauugagagcuaugaagaauucuuuuuauguggauauuuuaagaauuucgagcuaucugugcagucgaccaauuuaaacuaauaaguucuuuuuugauuaguuuaaauuggccuuuauuauuuauaaaaauuauauaaagaacaauauuuuagggguggaguaugaguaaauucgggaagaaaauauuaggaaugauguugguuauuuuaucaucuauagcuauaauauuaguuucaguaaaucugauuauguuugaaaaauuuaaaaaggaacuuaggcaaacaguuacucaauguauuucagauuuaacuguuuccauugauggagacaagcuugaaaagcuuauaaaagagcaaucagaugacaguguag

SEQ ID NO: 96 - RNA, 474 nt, Artificial Sequence
  synthetic construct; Cbe-1-2 c-di-GMP-II riboswitch aptamer
aauaaauauuguaagguaauuauugcauuuaauguugauuuuucuuauauuauuguuauaauauauacauagaaagacauaaaguaacauaaauauacaaagauugggaacuaugaagaauucucuguauuugggcaucuugagaauuuagaguuaaugaugcaacccaccaaucaaaacuaauuaauuuuuuuaauuaguuuugauugguuuuuuauauuuuggaauuaaacguaauagagcuaaaaguaaauaagauaguuuuuuuuaaguuaauauauaugagaggugauaugcuaugaagauacauaaugaaaucaugaaaguuauaaaugacaauuuggaaaaguguucuaaauucgaauuuguugcagaguuaagagauuuaacuuuagcagauauguauuauauugaaaagauaucgaguauugauaguauaaaagcgaaguucaauuauaaaauaauaaauaacac

SEQ ID NO: 97 - RNA, 474 nt, Artificial Sequence
  synthetic construct; Cbe-1-3 c-di-GMP-II riboswitch aptamer
ggaaugaaauucgacaauuuauauuaagaaucauugauuuucuuacaugaauaugauaauauuucuauauauuagguaaguguauuuauaaaaaauagaacaauuggaagcgaugaaaaauucucuauaucugggcacuuugagaauuuggagcuaguugugcaaccgaccaauuaaaacuuaguaauccauaaauauauuaaguuuuuguugguuuuuuauuuuuuauuaaucauuuuaauaaauauauuguaauaaugaaauuauauuuaagauccuaguaugaagauaagggggacaauaaaaugacguggcuuaauaauuuaaagguagguagaaaauuagcgauacuuauaaucugcucuuuaguaggauuauuugcaguuggaauaaccgguuacuauuuuuuacaaacgucuaguaaaaguauggagguuauguauagugaaagauugcuaucaagugaauggcuua

SEQ ID NO: 98 - RNA, 473 nt, Artificial Sequence
  synthetic construct; Cbe-1-4 c-di-GMP-II riboswitch aptamer
cuaauuuaguauucuuaaugauauuauuuguugauuuuauuauagauugaugauaauauuucauauauaugaaauaaguacauauuaauguauuauauuacaauuggaagcuaugaagaauucuuucuaagugggcacuuuaagaguuuggagcuaguugugcaaccgaccaauuaagauuaauuaauuauuuuauuaguuaguuuuaaugguuuuuuaugugaaaaaauuuacuugauuuauauauaaauaagaacuuauauaucuuaaguuuaagcauuacguaaguaagcucauugagagaagauuuuaauuuuuaucaaucacaauuauacuacagcuacaguaaggguuaaauuaagagguauauaaguauuaaugaaggggaugagacuguuauggauauuuuuguagcuagacagccuauauuuaacaaaaaaaaugagguuguugcauaugaacuucuauuuaga

SEQ ID NO: 99 - RNA, 476 nt, Artificial Sequence
  synthetic construct; Cdi-1-1 c-di-GMP-II riboswitch aptamer
ugucuuuuauuagauaaguaucuaggauacuaauuuauauuuuauugaguuguuaaaaaagugcacuuaaaaaguuaaaaaagaaaaauuaaaaaaccuauuuauagaaacugugaaguauaucuuaaaccugggcacuuaaaagauauauggaguuaguagugcaaccugcuauaaauauaaauaggaguaacuuuuaauugucaaaaauauauaaaauaaauacauaaauaaguaauuaauagaagauagauauaaauauauuuuugaaaugcucccugcauucauaacgaguguagaguggcagauaucugcccaaaacuccuuuaauugcuggaaguuccuaaagcuaacuaaaccacaacauaaucaugaaauaaggauaagugugauagagugugcauuuaauaagcacuugagguuacgaaaguagaaaaaauuaguuagauagcauaagguuaaauccuaagugccuu

SEQ ID NO: 100 - RNA, 475 nt, Artificial Sequence
  synthetic construct; Cdi-2-1 c-di-GMP-II riboswitch aptamer
aucuuauaucuaaaaauauggaaauauugacaaaauaauauuacaauguuagaauaaaaaagaaauuuaugaaaauauauaaaagauuuuuaauaaaaauauuuuagaaacugagaaguauaucuuauuauugggcaucuggagauauauggaguuaguggugcaaccggcuaugaauauaauuuuuauauugcauagucuuuuuuugcuuuaaaauuaacaaaaauguugcaaaauuugaaaauaagaguauuagucguuaagauuuuuauugauaggugaaauuuuggcuuuuaaaguagccauuuguuggaaaagggaaauuuuuuaaaaaguauaauacuauuauaauaagaaaauaaaguuuuuauaaacauugagaugcaaaauauaaaccuuuuguaacaauauauuuaccaaaauaauacaauuuauuuuauauuauaaccauauaaaucauauaaaucaauacuca

SEQ ID NO: 101 - RNA, 475 nt, Artificial Sequence
  synthetic construct; Cdi-1-2 c-di-GMP-II riboswitch aptamer
aucuuauaucuaagaauauggaaauauugacaaaauaauauuacaauguuagaauaaaaaagaaauuuaugaaaauauauaaaagauuuuuaauaaaaauauuuuagaaacugagaaguauaucuuauuauugggcaucuggagauauauggaguuaguggugcaaccggcuaugaauauaauuuuuauauugcauagucuuuuuuuguuuuaaaauuaacaaaaauguugcaaaauuugaaaauaagaguauuagucguuaagauuuuuauugauagguaaaauuuuggcuuuuaaaguagccauuuguuggaaaagggaaauuuuuuuaaaagaauaauaguauuauaauaagaaaauaaaguuuuuauaaacauugagaugcagaauauaaaccuuuuguaacaauauauuuaccaaaauaauacaauuuauuuuauauuauaaccauauaaaucauauaaaucaauacuca

SEQ ID NO: 102 - RNA, 476 nt, Artificial Sequence
  synthetic construct; Cdi-2-2 c-di-GMP-II riboswitch aptamer
auguaaaaaagauagacaauaguauagaggcauaauauaauagaaguauauaauuguauuuuuaauauaaauaaagcaauuuuaauuguuuuuaugaaauauuauagagauguugaaguauauucuauuauugggcaccuuauggauauacugagucaguggugcaaccggcuaugaauauaaauuuauuuauuuucauagcuuuuuuuuauauaauuuuuuaguaaaaauuaauuuauauacgauauuaauggaagauuauaaacuauagauaaccuagagggggaaaauuuauaugaagaaaggaaauaaaaaggcauuauuaauuaguuuaauuaugauuuuaaguaugguggugucuacaauauauccaacuguaucuuaugcuucagaauuaggagagaauagucagauucaaagugguucaacuaauucaucaacuggagaggagaaggaaagcgacaauaaaaaaccag

SEQ ID NO: 103 - RNA, 476 nt, Artificial Sequence
  synthetic construct; Cdi-3-2 c-di-GMP-II riboswitch aptamer
auguaaaaaagauagacaauaguauagaggcauaauauaauagaaguauauaauuguauuuuuaauauaaauaaagcaauuuuaauuguuuuuaugaaauauuauagagauguugaaguauauucuauuauugggcaccuuauggauauacugagucaguggugcaaccggcuaugaauauaaauuuauuuauuuucauagcuuuuuuuauauaauuuuuuaguaaaaauuaauuuauauacgauauuaauggaagauuauaaacuauagauaaccuagagggggaaaauuuauaugaagaaaggaaauaaaaaggcauuauuaauuaguuuaauuaugauuuuaaguaugguggugucuacaauauauccaacuguaucuuaugcuucagaauuaggagagaauagucagauucaaagugguucaacuaauucaucaacuggagaggagaaggaaagcgacaauaaaaaaccaga

SEQ ID NO: 104 - RNA, 476 nt, Artificial Sequence
  synthetic construct; Cdi-1-3 c-di-GMP-II riboswitch aptamer
auguaaaaaggauagacaauaguauagaggcauaauauaauagaaguauauaauuguauuuuuaauauaaauaaagcaauuuuaauuguuuuuaugaaauauuauagagauguugaaguauauucuauuauugggcaccuuauggauauacugagucaguggugcaaccggcuaugaauauaaauuuauuuauuuucauagcuuuuuuuguauaauuuuuuaguaaaaauuacuuuauauacgauauuaauggaagauuagauuauaaauuauagguaaccuaaaggggugagauuuacaugaagaaaggaaauagaaaggcauuauuaauuaguuuaauuaugauuuuaaguaugguggugucuacaauauauccaacuguaucuuaugcuucagaauuaggagagaauagucagauucaaagugguucaacuaauucaucaacuggagaggagaaggaaagcgacaauaaaaag

SEQ ID NO: 105 - RNA, 474 nt, Artificial Sequence
  synthetic construct; Cpe-7-1 c-di-GMP-II riboswitch aptamer
uuugacagguuuuuuaaaauacuguaacauacaaauacaaauugaaaauuaaguguuaaaaaguaguaauuuuuaagaaauaauuaguaaauauuaauauguuauagaaacguugauguauauucuguauuugggcaucuggaauaugcugaguuagugaugcaaccgacuauauuuauaaaaaguuauaaauauagucgguuuuuuauuuuccauuuuuuaagagaggagggcuuauuuuaugaagaauaguaaaaauuucaauauauuuaccuuauggucaguuguuauuucuaugauauuaauuaugauuucuccuggaagcauuguuuuaggagaagaaacaaaaucaguucaaaaugauggggcuguugaaauuacaagcacuaccuuugaaaguaauacuguugcuaaaggcauaaguaauaauuuaagaauugacuauaagauuuugaauaaagauaagcuuaaaga

SEQ ID NO: 106 - RNA, 474 nt, Artificial Sequence
  synthetic construct; Cpe-1-1 c-di-GMP-II riboswitch aptamer
uuugacaguuuuuuuaaaauacuguaacauacaaauacaaauugaaaauuaaguguuaaaaaguaaugaauuuuaagaaauaauuaguaaauauuaaaauguuauagaaacguugauguauauucuguauuugggcaucuggaauaugcugaguuagugaugcaaccgacuauauuuauaaaaaguuauaaauauagucgguuuuuuauuuuccauuuuuuaagagaggagggcuuauuuuaugaagaauaguaaaaauuucaauauauuuaccuuauggucaguuguuauuucuaugauaguaauuaugaucuucccuggaagcauuguuuuaggagaagaaucaaaaucaguucaaaaugauggggcuguugaaauuacaagcacuacuuuugagaguaauacuguggcuaaagacauaagcaauaauuuaagaauugacuauaagauuuugaauaaagauaagcuuaaaga

SEQ ID NO: 107 - RNA, 474 nt, Artificial Sequence
  synthetic construct; Cpe-2-1 c-di-GMP-II riboswitch aptamer
uuugacaguuuuuuuaaaauacuguaacauacaaauacaaauugaaaauuaaguguuaaaaaguaaugaauuuuaagaaauaauuaguaaauauuaaaauguuauagaaacguugauguauauucuguauuugggcaucuggaauaugcugaguuagugaugcaaccgacuauauuuauaaaaaguuauaaauauagucgguuuuuuauuuuccauuuuuuaagagaggagggcuuauuuuaugaagaauaguaaaaauuucaauauauuuaccuuauggucaguuguuauuucuaugauaguaauuaugaucuucccuggaagcauuguuuuaggaaaagaagcaaaaucaguucaaaaugauggggcuguugaaauuacaagcacuaccuuugaaaguaauacuguugcuaaagacauaagcaauaauuuaagaauugauuauaagauuuugaauaaagauaagcuuaaaga

SEQ ID NO: 108 - RNA, 474 nt, Artificial Sequence
  synthetic construct; Cpe-8-1 c-di-GMP-II riboswitch aptamer
uuugacaguuuuuuuaaaauacuguaacauacaaauacaaauugaaaauuaaguguuaaaaaguaaugaauuuuaagaaauaauuaguaaauauuaaaauguuauagaaacguugauguauauucuguauuugggcaucuggaauaugcugaguuagugaugcaaccgacuauauuuauaaaaaguuauaaauauagucgguuuuuuauuuuccauuuuuuaagagaggagggcuuauuuuaugaagaauaguaaaaauuucaauauauuuaccuuauggucaguuguuauuucuaugauauuaauuaugauuucuccuggaagcauuguuuuaggagaagaaacaaaaucaguucaaaaugauggggcuguugaaauuacaagcacuaccuuugaaaguaauacuguugcuaaaggcauaagcaauaauuuaagaauugacuauaagauuuugaauaaagauaagcuuaaaga

SEQ ID NO: 109 - RNA, 474 nt, Artificial Sequence
  synthetic construct; Cpe-4-1 c-di-GMP-II riboswitch aptamer
uuugacaguuuuuuuaaaauacuguaacauacaaauacaaauugaaaauuaaguguuaaaaaguaaugaauuuuaagaaauaauuaguaaauauuaaaauguuauagaaacguugauguauauucuguauuugggcaucuggaauaugcugaguuagugaugcaaccgacuauauuuauaaaaaguuauaaauauagucgguuuuuuauuuuccauuuuuuaagagaggagggcuuauuuuaugaagaauaguaaaaauuuuaauauauuuaccuuauggucaguuguuauuucuaugauaguaauuaugaucuuuccuggaagcauuguuuuaggagaagaaacaaaaucaguucaaaaugauggagcuguugaaauuacaagcacuacuuuugagaguaauacuguggcuaaagacauaagcaauaauuuaagaauugacuauaagauuuugaauaaagauaagcuuaaaga

SEQ ID NO: 110 - RNA, 476 nt, Artificial Sequence
  synthetic construct; Cdi-1-4 c-di-GMP-II riboswitch aptamer
uauauaaauaguauaaacaguguugcuaaaauaugguagauauuauauaauauaaguggauauguauauacauuaugaguuauauaauuuugaauuuaauaaaauagaaacguugauuuauguucuguaaugugggcaccuuggagcauauugaguuaguggugcaaccggcuaugaaauuguauuuauuuauagauacuauuauuuucauagcuuuuuuuauuuaaaaaaugguuauauguuagaaauaaauuugguuaguacguuuaaauaauuuuacuuagauuuuuaaccagauauauuauaaguaaguauuaauuaaaaaauuuaagggggaauaaaaaugaaguuaaaaaagaauaaaaaagguuucacuuuaguggaauuauugguaguaauugcaauuauagguauauuagcaguaguggcaguuccagcuuuauuuaguaauauaaacaaggcuaagguagcaagug

SEQ ID NO: 111 - RNA, 476 nt, Artificial Sequence
  synthetic construct; Cdi-2-3 c-di-GMP-II riboswitch aptamer
uauauaaauaguauaaacaguguugcuaaaauaugguagauauuauauaauauaaguggauauguauauacauuaugaguuauauaauuuugaauuuaauaaaauagaaacguugauuuauguucuguaaugugggcaccuuagagcauauugaguuaguggugcaaccggcuaugaaauuguauuuauuuauagauacuaucauuuucauagcuuuuuuuauuuaaaaaaugguuauguguuagaaauaaauuugguuaguacguuuaaauaauuuuacuuagauuuuuaaccagauauauuauaaguaaguauuaauuaaaaaauuuaagggggaauaaaaaugaaguuaaaaaagaauaaaaaagguuucacuuuaguggaauuauugguaguaauugcaauuauagguauauuagcaguaguggcaguuccagcuuuauuuaguaauauaaacaaggcuaagguagcaagug

SEQ ID NO: 112 - RNA, 474 nt, Artificial Sequence
  synthetic construct; Cac-1-1 c-di-GMP-II riboswitch aptamer
auaaaaaauauuaauuuuuuuugauuuaccuauuaaagaaaauaaaaacuauacgaaaauaauauuguuaagaguuuuaaauaagguaacaaaaaauuaagcguuggagacugugaagaauuucuuguauuugggcaccuuagaaauuuggaguuaguggugcaaccugccaacaauaauuagagaauuaauuauuggaggaguggcuaaaugaaacuuuuuaaaacccuuauucuuaaaucaaauuugaaaagacuaggaggugggcacuuaggaacccaaaauuuugauguuuuuucuaaguuuauagguuuaugucuaucuuuaaaauauuucauauuaaaauauaagagcaaauuuuauaaugaagauaagauaaauaaauuagauggaaacauugauacacaugauucggauauaaaaaguauaagagaauuuaaugagaguucagauauaguaagagaagauucagua

SEQ ID NO: 113 - RNA, 474 nt, Artificial Sequence
  synthetic construct; Cno-1-1 c-di-GMP-II riboswitch aptamer
aauucuuuauuauuaauaaaauauauauuaaaauuuuauauugacaaaguaauaugcauaaauuacuauaaauauaagauuaacaaaauauuuacuuuuuaaaaggagacauuugaacuauauacuuuauuugggcacuuuguauauagggagucauuagugcaaccgaccuuauuuuauuuagggucgguuuuuauuuuuuuuaguaauaugguuaauauuguuaauuggguuuuuguauuaaauuaaauauuauaggggggaacauauuggaugaaccuaugaaaguuaauuugaaaaauauuaugaaacugauauuuuuauuuauaauuuuucaaauaugcuuauucauauuuuucaacaaaaguacgguugcauuugcaaaagauuuagauguuauaaaaagcauaaaaauaacuaaugaaaauggacaaaauaaagaaacuuacauuccaggugauagaauuaggguug

SEQ ID NO: 114 - RNA, 473 nt, Artificial Sequence
  synthetic construct; Cte-1-1 c-di-GMP-II riboswitch aptamer
aaaugguuaaauuuauuaccuaauacucauuauugauaaaaucaugaaauuaaaguaauauguauauacaauaguuuaauaccauauaaauauaauauuuuaagaggaaauuuugaacuauauacuuauuugggcacuuuguauauagggaguuaguagugcaaccgaccuugauuaaucagggucgguuuuuauuuuuaaauaaagcaagaaauaacuaggaggggguauauugaaaaccuuugacaaaauaauuaaaggcagaaaaaauaucauaauuauuuugacguuuuuauuucagauauuauuaugcucaaaaacuguauuagcagcagauguagauguuauagauaauguuaaaauaacuaaugaaaaggggguagugcaaggugguuauagaccaggugauagaauaaaaauugauguagauuggucuauuaaaucagaaacaaaagcuggagauaccuuuaguu

SEQ ID NO: 115 - RNA, 474 nt, Artificial Sequence
  synthetic construct; Cbe-1-5 c-di-GMP-II riboswitch aptamer
uagaccaauuaaugaucauuaugguauaauuacuauggcauauauuggcuacauuuaauauguuaugcaagaaauaaaagaaauaacaaguauauuuugucauaaggaaacuuugauccauauauuuuaauugggcacuuuauauauggugaguuaguagugcaaccgaccuuuauauuuuauaaaggccgguuuuuauauuuuuuaaauaaaaguaugaaauauuuuuagguaagacagaggaagauauugaaauacaaucagauuaacuguauuuuaauaaaaagaaaguuuauuauguuuauauaauuccuauuaaauauuuaaaaaaauguaguuaaaauacaaauuuguacauaacaacguuuacaaaaaaguuaaauaauauuaaaauauacaaugagaggugauuuuuuggauuauauauuuauauguauuuugggacuaguuauagguaguuuuuuaaauguuugu

SEQ ID NO: 116 - RNA, 473 nt, Artificial Sequence
  synthetic construct; Cbe-1-6 c-di-GMP-II riboswitch aptamer
auauguuaaaauuugaaauuguuucauuuuauucacuauuuguucauugauuuguaauuaauuccauguuaaaaugaucauuaaauaguaauauuauguauauaaggaaacugugauucgaauauuuacuugggcacuuuauauucgaugaguuaauagugcaaccgaccuuuuuauuaaggucggucuuuuuucguccgcuuaaauaaguauauagauaauaaauuaaaauugaggugcaaucuuuauguuauuuuuuaauauguuaaagaaauaugaugaacauggguuugauucaaaaggaauucauaaaaacgguacaaaauuuaaugaagaggguuuugauaaaaaagggguucauaaaaauggcacauauuuuaauagagaaggcuacaauauagacggguacgauaaauauggauaugauaaagaaggauauaauaguggaggauaugacaggcaaggauauaaca

SEQ ID NO: 117 - RNA, 474 nt, Artificial Sequence
  synthetic construct; Cac-1-2 c-di-GMP-II riboswitch aptamer
caauuuuaagguuuuagcuaugaaaaaguagucuagaggcagguugcuuuaaauauaauuuugugguaaaauauaacuauuaauauaaagaauaccuauuuguuuggaaacaaugaugaauuucuuuaaauugggcacuugagaaauuuugaguuaguagugcaaccgaccaacgauuaauuaagcuguauuaauuguugguuuuuugcuuguguaaggagguguuuaaacuauuaaaguauaaaaauguaauuuauguauuauuguucaauauaauuuacauaauagggauauaagauguagagagguuaaauaaaauaauaguuuauaaaugccaagcaucauauuuuaauauuaaaaagauauaauguuaauucguuuuucaacuauaguggcuaaauugagaauuaugaagauaaauguaggaaaaucaauuugauuuuccuacauuuguuuuuugaaaagguaaauaua

SEQ ID NO: 118 - RNA, 475 nt, Artificial Sequence
  synthetic construct; env-8 c-di-GMP-II riboswitch aptamer
uacguaccgcacgccugaagaacuuguuguaacgcagacaucguaacugugacagugcgagugcuagccuucggucgugggucccgcauguggaccucgcgauucgagggcgaugacgcucguucugcaacaugggcgcuuggaacgagccgagccaguagcgcaaccgaucggucgacguucucaucgugauucgcacuccgccuucuccgaucuccccuccacccgaaacggucaagcacccaaucguuggacgccuccugcgaggcguccggaggcgaugacccgcugauccccauuugggcaucaagggacgcggcgagccagugaugcaaccggccggacggcuacgcacguguguacguggccccaggccucggaggaggaacgaugcaagaccucgcacgcccgcggagcggccugcuccgcaaccugccgcgcacgauccuguucgcgcucggccucaucggauuac

SEQ ID NO: 119 - RNA, 475 nt, Artificial Sequence
  synthetic construct; env-9 c-di-GMP-II riboswitch aptamer
ucuucaagaggccgagggagcgaccagcugaagcuggccggaccaagcguucaggacgcancunggcanccggguuunugggnggnaaagccgcccucacccuucagaggcuuagaugccuaccccgcaacuugggcgcuugggguacgcggagccaguagcgcaaccggccgagggcuuccuuuugcuuccucacaucccaggcuuugagggucccuuagguagucauggcagccccaugccuaccggaaugcgaggaguacgugaggaacgcgaacaacgcacauggcacgucagcaccgcucaggcguucgaggagccugcuggcagugcuauccgcccuggugcugguacuagcggccuguucgacgccagccgaagaaccgaugccgcagcccgaguugacgacggaugacaucgucuucuccgccgcaacaagcagcacaacggagaucaagcagcucaaggacagcac

SEQ ID NO: 120 - RNA, 475 nt, Artificial Sequence
  synthetic construct; Tca-1-1 c-di-GMP-II riboswitch aptamer
uacaugaauuaacaguucuuugaaauauuguaaacccgucuacuucauugcaggauuuuuaccaauuuauuacgaauauauacguagucaaaacaauacugcaaugggugugaugaaguccggacaguaaugugggcacuuaguccggaccgagcaaguagugcaaccgaccagaugcaaaauaacguuuuugcuguuggcgguuguuuuucguuuuugggggcgggcauggugaaaacacggaaacgcuugggcgauuugcugcuugaggcgggucucauaacgccugaacagcuugaaaaagcccuuaaugugcaaaaaaagacgggggaacggcuggggaaaguucugaucaaccucggguauauaacugaggacaguaugauagagguccuugaauuccagcugggaguuccucauaucgacuugggaagcgugccgcccgauccggaggcagcggcgacuauuccggcuu

SEQ ID NO: 121 - RNA, 509 nt, Artificial Sequence
  synthetic construct; consensus sequence for c-di-GMP-II riboswitches
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnrgarrcnnnugannyrynnnnnyunnnaynuggrcnacyunnnnnnnnnnnnnnnnnnnnnnnnnnnrnnnryrnnngagyyagurgugcaaccgrcynnnnnnnnnnnnnnnnnnnnnynnnnnnnnnnynnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnn

SEQ ID NO: 122 - RNA, 864 nt, Clostridium difficile
gguaaaaaaccuauuuauagaaacugugaaguauaucuuaaaccugggcacuuaaaagauauauggaguuaguagugcaaccugcuauaaauauaaauaggaguaacuuuuaauugucaaaaauauauaaaauaaauacauaaauaaguaauuaauagaagauagauauaaauauauuuuugaaaugcucccugcauucauaacgaguguagaguggcagauaucugcccaaaacuccuuuaauugcuggaaguuccuaaagcuaacuaaaccacaacauaaucaugaaauaaggauaagugugauagagugugcauuuaauaagcacuugagguuacgaaaguagaaaaaauuaguuagauagcauaagguuaaauccuaagugccuugaaaauggauaaucagcagguaagcuucgaaaagaagaaacuucaacgacuagagagaaaucucguaccuacaaguguggggaaauggggaguaccuaauuuaugguuagguaaugauauagucugugcuuaauagaaauauuaagaaguucauaggagaacugguuaggaguaacgacccuaauugaacacacuaaagauguauuauugaguaagauucauauaauauuuaauaauuuuaauuauauuaagugaauacaauauauguuuuuaguaucaggagaauuuugaaaacaaaaauuaaaaaaucaaguauaaucaguuuaguaauugcuuuuaguaugguauuuacugcauuuacaccaauaguaucuuaugcugacgaagugacuagcaaugauacaauacuuaauggagaagaacaaucaaauggucagacaccagauguagagaaaccaucagacggucagguacc